(19) United States
(12) Patent Application Publication
(10) Pub. No.: US 2014/0088056 A1
(43) Pub. Date: Mar. 27, 2014

(54) CARDIAC GLYCOSIDES ARE POTENT INHIBITORS OF INTERFERON-BETA GENE EXPRESSION

(71) Applicants: Junqiang Ye, Fort Lee, NJ (US); Shuibing Chen, Pelham, NY (US); Tom Maniatis, New York, NY (US)

(72) Inventors: Junqiang Ye, Fort Lee, NJ (US); Shuibing Chen, Pelham, NY (US); Tom Maniatis, New York, NY (US)

(73) Assignee: PRESIDENT AND FELLOWS OF HARVARD COLLEGE, Cambridge, MA (US)

(21) Appl. No.: 13/876,795

(22) PCT Filed: Sep. 28, 2011
(86) PCT No.: PCT/US2011/053652

§ 371 Date: Nov. 25, 2013

Related U.S. Application Data
(60) Provisional application No. 61/387,407, filed on Sep. 28, 2010.

(51) Int. Cl. A61K 31/585; A61K 31/519; A61K 31/17; A61K 31/505; A61K 31/407; A61K 31/136

(52) U.S. Cl. CPC A61K 31/585; A61K 31/407; A61K 31/136; A61K 31/17; A61K 31/505; A61K 31/519
USPC 514/175; 514/410; 514/656; 514/597; 514/256; 514/264.11; 435/375; 435/184

(57) ABSTRACT

The invention provides for a method of inhibiting interferon-beta gene expression and/or reducing the level of interferon-beta in a cell by contacting the cell with a Na⁺, Ca²⁺, or K⁺ ion-channel modulator. The invention also provides for a method of treating a disease or disorder characterized by elevated interferon beta levels or elevated levels of interferon-beta gene expression. Additionally, the invention provides a method for treating pathogenic or non-pathogenic infections.

### RELATED APPLICATIONS

This application claims benefit under 35 U.S.C. §119(e) of the U.S. Provisional Application No. 61/387,407, filed Sep. 28, 2010, content of which is incorporated herein by reference in its entirety.

### GOVERNMENT SUPPORT

This invention was made with government support under grant no. 5R01AI020642-26 awarded by the National Institutes of Health. The government has certain rights in the invention.

### FIELD OF THE INVENTION

The invention relates to methods and compositions for inhibiting interferon-beta gene expression.

### BACKGROUND OF THE INVENTION

The production of Type I interferons (IFN) is induced in virtually every cell type by virus infection, double stranded RNA or DNA (dsRNA and DNA). See for example, Sen, G. C. Viruses and interferons. Annu Rev Microbiol 55, 255-81 (2001); Honda, K., Takaoka, A. & Taniguchi, T. Type I interferon [corrected] gene induction by the interferon regulatory factor family of transcription factors. Immunity 25, 349-60 (2006); Chiu, Y. H., Macmillan, J. B. & Chen, Z. J. RNA polymerase III detects cytosolic DNA and induces type I interferons through the RIG-I pathway. Cell 138, 576-91 (2009); and Ablasser, A. et al. RIG-1-dependent sensing of poly(dA:dT) through the induction of an RNA polymerase III-transcribed RNA intermediate. Nat Immunol 10, 1065-72 (2009). Type I interferons, in turn, induce the expression of hundreds of interferon stimulated genes (ISGs) that encode antiviral activities. These activities coordinate the establishment of a strong antiviral environment within the cell (Garcia-Sastre, A. & Biron, C. A. Type 1 interferons and the virus-host relationship: a lesson in detente. Science 312, 879-82 (2006)). Type I interferon also plays and essential role in the activation of immune cell activity in both the innate and adaptive immune responses. See, for example, Garcia-Sastre, A. & Biron, C. A. Type 1 interferons and the virus-host relationship: a lesson in detente. Science 312, 879-82 (2006); Le Bon, A. et al. Type i interferons potently enhance humoral immunity and can promote isotype switching by stimulating dendritic cells in vivo. Immunity 14, 461-70 (2001); and Le Bon, A. & Tough, D. F. Links between innate and adaptive immunity via type I interferon. Curr Opin Immunol 14, 432-6 (2002).

While essential for the elimination of infectious agents, high levels of IFN can be toxic. In fact, over-expression or aberrant expression of IFN has been implidated in several inflammatory and autoimmune diseases. See for example, Banchereau, J. & Pascual, V. Type I interferon in systemic lupus erythematosus and other autoimmune diseases. Immunity 25, 383-92 (2006); Yoshida, H., Okabe, Y., Kawane, K., Fukuyama, H. & Nagata, S. Lethal anemia caused by interferon-beta produced in mouse embryos carrying undigested DNA. Nat Immunol 6, 49-56 (2005); Yarilina, A. & Ivashkiv, L. B. Type I Interferon: A New Player in TNF Signaling. Curr Dir Autoimmun 11, 94-104; and Hall, J. C. & Rosen, A. Type I interferons: crucial participants in disease amplification in autoimmunity. Nat Rev Rheumatol 6, 40-9. Overproduction of interferon has been recognized as the major cause of systemic lupus erythematosus (SLE) (Banchereau, J. & Pascual, V. Type I interferon in systemic lupus erythematosus and other autoimmune diseases. Immunity 25, 383-92 (2006)). In addition, strong innate immune responses (including IFN production) have been shown to contribute to AIDS virus infection (Mandl, J. N. et al. Divergent TLR7 and TLR9 signaling and type I interferon production distinguish pathogenic and nonpathogenic AIDS virus infections. Nat Med 14, 1077-87 (2008)). Regulating the levels and duration of IFN production is critical to the optimization of antiviral activities, while minimizing the detrimental effects associated with over-production or prolonged expression. Normally, IFN is transiently expressed after infection. See for example, Whittemore, L. A. & Maniatis, T. Post induction turnoff of beta-interferon gene expression. Mol Cell Biol 10, 1329-37 (1990); Raj, N. B., Cheung, S. C., Rosztoczy, I. & Pitha, P. M. Mouse genotype affects inducible expression of cytokine genes. J Immunol 148, 1934-40 (1992); Pandos, M., Shimonaski, G. & Came, P. E. Interferon in mice acutely infected with M-P virus. J Gen Virol 13, 163-5 (1971); and Jacquelin, B. et al. Nonpathogenic SW infection of African green monkeys induces a strong but rapidly controlled type I IFN response. J Clin Invest 119, 3544-55 (2009).

The activation of IFNβ gene expression is one of the most extensively studied gene regulatory systems (Maniatis, T. et al. Structure and function of the interferon-beta enhanceosome. Cold Spring Harb Symp Quant Biol 63, 609-20 (1998); Kawai, T. & Akira, S. Innate immune recognition of viral infection. Nat Immunol 7, 131-7 (2006); Akira, S., Uematsu, S. & Takeuchi, O. Pathogen recognition and innate immunity. Cell 124, 783-801 (2006); and Honda, K., Takaoka, A. & Taniguchi, T. Type I interferon [corrected] gene induction by the interferon regulatory factor family of transcription factors. Immunity 25, 349-60 (2006). Virus infection triggers the activation of a complex signal transduction pathway (Sun, L., Liu, S. & Chen, Z. J. SnapShot: pathways of antiviral innate immunity. Cell 140, 436-436 e2)) leading to the coordinate activation of multiple transcriptional activator proteins that bind to the IFNβ enhancer to form an enhanceosome, which recruits the transcription machinery to the gene (Maniatis, T. et al. Structure and function of the interferon-beta enhanceosome. Cold Spring Harb Symp Quant Biol 63, 609-20 (1998) and Ford, E. & Thanos, D. The transcriptional code of human IFN-beta gene expression. Biochim Biophys Acta 1799, 328-336). The presence of viral RNA is detected by the RNA helicases RIG-I and MDA5 (they appear to have specificity for different viruses). See for example, Yoneyama, M. & Fujita, T. Structural mechanism of RNA recognition by the RIG-I-like receptors. Immunity 29, 178-81 (2008) and Kato, H. et al. Differential roles of MDA5 and RIG-I helicases in the recognition of RNA viruses. Nature 441, 101-5 (2006). Upon binding RNA RIG-I or MDA5 dimerize, undergo a conformation change and expose a critical N-terminal caspase recruiting domain (CARD) (Cui, S. et al. The C-terminal regulatory domain is the RNA 5′-triphosphate sensor of RIG-I. Mol Cell 29, 169-79 (2008) and Takahasi, K. et al. Nonself RNA-sensing mechanism of RIG-I helicase and activation of antiviral immune responses. Mol Cell 29, 428-40 (2008)) that binds to a corresponding CARD domain in the downstream adaptor protein MAVS on the mitochondria membrane (Seth, R. B., Sun, L., Ea, C. K. & Chen, Z. J. Identification and characterization of MAVS, a mitochondrial antiviral signaling protein that activates NF-kappaB and IRF 3. Cell 122, 669-82 (2005); Kawai, T. et al. IPS-1, an adaptor triggering RIG-I- and Mda5-mediated type I interferon induction. Nat Immunol 6, 981-8 (2005); Xu, L. G. et al. VISA is an adapter protein required for virus-triggered IFN-beta signaling. Mol Cell 19, 727-40 (2005); and Meylan, E. et al. Cardif is an adaptor protein in the RIG-I antiviral pathway and is targeted by hepatitis C virus. Nature 437, 1167-72 (2005)). MAVS is also believed to form dimers on the surface of mitochondria (Tang, E. D. & Wang, C. Y. MAVS self-association mediates antiviral innate immune signaling. J Virol 83, 3420-8 (2009) and Baril, M., Racine, M. E., Penin, F. & Lamarre, D. MAVS dimer is a crucial signaling component of innate immunity and the target of hepatitis C virus NS3/4A protease. J Virol 83, 1299-311 (2009)), leading to the further recruitment of downstream signal molecules and kinases. The assembly of these signaling components ultimately leads to the activation of the key transcription factors Interferon Regulatory Factors IRF3/7 and NFκB. Phosphorylated IRF3/7 and NFκB translocate into the nucleus, together with activated cJUN and ATF2, to form the enhanceosome complex including CBP/p300 on the promoter of the IFNb gene (Maniatis, T. et al. Structure and function of the interferon-beta enhanceosome. Cold Spring Harb Symp Quant Biol 63, 609-20 (1998)). Histone modification and chromatin remodeling enzymes and RNA polymerase machinery are recruited to drive the transcription of the IFNβ gene. See, Agalioti, T. et al. Ordered recruitment of chromatin modifying and general transcription factors to the IFN-beta promoter. Cell 103, 667-78 (2000) and Agalioti, T., Chen, G. & Thanos, D. Deciphering the transcriptional histone acetylation code for a human gene. Cell 111, 381-92 (2002).

The initial trigger of the IFN signaling pathway is the recognition of viral RNA. Recently, short double strand RNA (dsRNA) or panhandle RNA with 5′-ppp group has been shown to be the RNA structure that activates RIG-I (Kato, H. et al. Length-dependent recognition of double-stranded ribonucleic acids by retinoic acid-inducible gene-I and melanoma differentiation-associated gene 5. J Exp Med 205, 1601-10 (2008); Schlee, M. et al. Recognition of 5′ triphosphate by RIG-I helicase requires short blunt double-stranded RNA as contained in panhandle of negative-strand virus. Immunity 31, 25-34 (2009); Schmidt, A. et al. 5′-triphosphate RNA requires base-paired structures to activate antiviral signaling via RIG-I. Proc Natl Acad Sci USA 106, 12067-72 (2009); and Fujita, T. A nonself RNA pattern: tri-p to panhandle. Immunity 31, 4-5 (2009)). RIG-I dimerizes upon binding RNA (Cui, S. et al. The C-terminal regulatory domain is the RNA 5′-triphosphate sensor of RIG-I. Mol Cell 29, 169-79 (2008) and Takahasi, K. et al. Nonself RNA-sensing mechanism of RIG-I helicase and activation of antiviral immune responses. Mol Cell 29, 428-40 (2008)), and the dimer travels along the RNA, acting as a translocase (Myong, S. et al. Cytosolic viral sensor RIG-I is a 5′-triphosphate-dependent translocase on double-stranded RNA. Science 323, 1070-4 (2009)). This activity has been shown to be helicase dependent (Myong, S. et al. Cytosolic viral sensor RIG-I is a 5′-triphosphate-dependent translocase on double-stranded RNA. Science 323, 1070-4 (2009) and Saito, T. et al. Regulation of innate antiviral defenses through a shared repressor domain in RIG-I and LGP2. Proc Natl Acad Sci USA 104, 582-7 (2007)). Thus RNA binding and the helicase dependent translocation along the RNA template are two critical activities of RIG-I protein. Recent studies have revealed that RIG-I undergoes covalent modifications upon activation, its ubiquitination at lysine 172 by the E3 ligase Trim25 is important for signaling (Gack, M. U. et al. TRIM25 RING-finger E3 ubiquitin ligase is essential for RIG-I-mediated antiviral activity. Nature 446, 916-920 (2007)), while phosphorylation of threonine 170 by an unidentified kinase antagonizes RIG-I activation (Gack, M. U., Nistal-Villan, E., Inn, K. S., Garcia-Sastre, A. & Jung, J. U. Phosphorylation-mediated negative regulation of RIG-I anti-viral activity. J Virol.).

The activated RIG-I protein relays a signal to the mitochondria protein MAVS through CARD domains on both proteins. Since there is little mitochondria association of RIG-I after virus infection, the interaction between RIG-I and MAVS must happen transiently, and MAVS is able to efficiently assemble the downstream signaling complex. As the adaptor proteins, TRAF3, TRAF5, TRAF6 and TANK are thought to interact with MAVS, and activate the downstream kinases TBK1 and/or IKKe (Oganesyan, G. et al. Critical role of TRAF3 in the Toll-like receptor-dependent and -independent antiviral response. Nature 439, 208-11 (2006); Hacker, H. et al. Specificity in Toll-like receptor signalling through distinct effector functions of TRAF3 and TRAF6. Nature 439, 204-7 (2006); Saha, S. K. et al. Regulation of antiviral responses by a direct and specific interaction between TRAF3 and Cardif. EMBO J. 25, 3257-63 (2006); and Guo, B. & Cheng, G. Modulation of the interferon antiviral response by the TBK1/IKKi adaptor protein TANK. J Biol Chem 282, 11817-26 (2007)), as well the IKKα/β kinases (Seth, R. B., Sun, L., Ea, C. K. & Chen, Z. J. Identification and characterization of MAVS, a mitochondrial antiviral signaling protein that activates NF-kappaB and IRF 3. Cell 122, 669-82 (2005) and Kawai, T. et al. IPS-1, an adaptor triggering RIG-I- and Mda5-mediated type I interferon induction. Nat Immunol 6, 981-8 (2005)). Additional proteins have been reported to play roles in the activation of the IFN gene, including Sting/Mita, DDX3. See for example, Ishikawa, H. & Barber, G. N. STING is an endoplasmic reticulum adaptor that facilitates innate immune signalling. Nature 455, 674-8 (2008); Zhong, B. et al. The adaptor protein MITA links virus-sensing receptors to IRF3 transcription factor activation. Immunity 29, 538-50 (2008); and Schroder, M., Baran, M. & Bowie, A. G. Viral targeting of DEAD box protein 3 reveals its role in TBK1/IKKepsilon-mediated IRF activation. EMBO J 27, 2147-57 (2008). These proteins are thought to mediate interactions with RIG-I, MAYS or TBK1 proteins.

### SUMMARY OF THE INVENTION

The inventors have discovered that interferon gene expression can be regulated by modulating intracellular ion concentrations. Accordingly, in one aspect the invention provides a method for inhibiting induction of interferon-beta gene expression in a cell and/or reducing the secretion of interferon-beta from a cell, the method comprising contacting a cell with a Na, Ca²⁺, or K⁺ ion-channel modulator. In some embodiments of the aspects described herein, the modulator does not significantly modulate an amiloride-sensitive sodium channel. In some embodiments of the aspects described herein, the modulator is not an amiloride or analog or derivative thereof. In some embodiments, the modulator is bufalin or an analog, a derivative, a pharmaceutically acceptable salt, and/or a prodrug thereof.

In another aspects the invention provides a method for treating a subject suffering from a disease or disorder characterized by elevated levels of interferon-beta, the method comprising administering an effective amount of a Na⁺, Ca²⁺, or K⁺ ion-channel modulator to the subject. In some embodiments of the aspects described herein, the modulator does not significantly modulate an amiloride-sensitive sodium channel. In some embodiments of the aspects described herein, the modulator is not an amiloride or analog or derivative thereof. In some embodiments, the modulator is bufalin or an analog, a derivative, a pharmaceutically acceptable salt, and/or a prodrug thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E show Bufalin potently blocks virus, double strand RNA and DNA induced gene expression. FIG. 1A, Bufalin blocks virus induced IFNβ expression in reporter assays. 293T cells were transfected with the IFNβ promoter driving a firefly luciferase reporter together a reference renilla luciferase reporter plasmid. 24 hrs later, cells were treated with increasing amounts of bufalin (1 nM to 1 uM) and subsequently infected with sendai virus. Firefly luciferase activities were measured after another 24 hrs and normalized to that of renilla activities. FIG. 1B, Bufalin potently blocks the induction of PRDIII/I and PRDII elements of the IFNβ promoter. Experiments were conducted same as in A, but PRDIV, PRDIII/I and PRDII driving luciferase reporter plasmids were used instead. FIG. 1C, Microarray analysis demonstrating bufalin blocked the virus induced gene expression program. 293T cells were treated with bufalin or SeV alone, or in combination for 8 hrs, and total cellular RNA were extracted and subjected to Illumina Beadchip microarray analysis. Top lists of genes induced or repressed by Bufalin alone, or induced by SeV are shown. FIG. 1D, semi-quantitative RT-PCR confirming the microarray results of representative genes. FIG. 1E, Bufalin also potently inhibits gene induction by dsRNA and dsDNA. 293T cells were treated with bufalin (1 uM), and then subjected to poly I:C (dsRNA) or poly dA:dT (dsDNA) transfection, total cellular RNA were prepared after 6 hrs and subjected to RT-PCR analysis.

FIGS. 2A-2D shows Bufalin inhibits virus induced IRF3 and p65 activation. FIG. 2A, Bufalin does not destroy sendai virus pathogen-associated molecular pattern (PAMP). RNA samples from FIG. 1C were transfected into new 293T cells, 6 hrs later, cellular RNA were prepared and subjected to RT-PCR analysis with primers specific for IFNβ and CXCL10 and GAPDH. FIG. 2B, Bufalin blocks IRF3 dimerization. 293T cells were treated with bufalin or SeV, either alone or in combination for 6 hrs. Total protein was prepared and subjected to a native gel analysis for IRF3 dimerization. FIG. 2C, Bufalin blocks the virus induced nuclear translocation of both IRF3 and p65. 293T cells were treated same as in B, and cells were fixed with formaldehyde and immunofluoresence staining of IRF3 and p65 were conducted. FIG. 2D, Over-expression of RIG-I, MAVS and TBK1 greatly relieved the blockage of bufalin of the IFNβ induction. 293T cells were transfected with RIG-I, MAVS and TBK1 expression plasmids together with IFNβ promoter driving luciferase reporter in the presence or absence of bufalin, and infected with SeV infection for 24 hrs before measuring the luciferase activities. GFP plasmid was included as a control.

FIG. 2E shows that bufalin strongly inhibits IFNβ induction in cells preinfected with virus. 293T cells were first infected with SeV (200 HAU/ml) and 1.5 hours late, virus containing medium was replaced by fresh medium with or without the addition of bufalin (to a final concentration of 1 μM) and further incubated for 6 hours. Total RNA was extracted for the analysis of IFNβ, CCL5, RIG-I, and beta-actin expression by RT-PCR.

FIGS. 3A and 3C show that RIG-I helicase activity is inhibited by bufalin treatment. FIG. 3A, Bufalin does not affect the RNA binding ability of RIG-I. 293T cells stably expressing RIG-I protein were transfected with biotin-labeled dsRNA (67 bp, corresponding to 3′ end of GFP gene) in the presence or absence of bufalin, 6 hrs later, total cellular protein was prepared and subjected to NeutrAvidin beads binding. Bound RIG-I protein was analyzed by western blot (top panel). The expression of IFNβ and Cxcl 10 genes in these cells was also analyzed by RT-PCR (bottom panel). FIG. 3B, high salt concentration inhibits RIG-I helicase activities while the effect on RNA binding is minor. Recombinant RIG-I protein was incubated with dsRNA (67 bp) in the presence of increasing NaCl and KCl concentrations. RNA binding was monitored by native agarose gel analysis (top panel). For the ATPase activities, samples were adjusted to 1 mM ATP and further incubated for 15 min at 37° C., free phosphate released was measured with BIOMOL GREEN reagent. The image of the plate was shown in the bottom panel, and signals from the reading were quantified and graphed in the middle panel. The lanes of the gel, bars of the graph and wells in the ATPase assay are aligned according to experimental conditions. FIG. 3C, bufalin treatment increased the intracellular sodium concentration within 293T cells. 293T cells were loaded with 10 μM SBFI-AM in the presence of 0.02% Pluronic F-127 for 1 hour at 37° C. Cells were washed and treated with or without 1 μM bufalin for 30 minutes, fluorescence emission at 525 nm from 340 nm and 380 nm excitation were recorded and the ratio determined. To generate the standard curve, SBFI-Am loaded cells were exposed to solutions with increasing concentrations of sodium in the presence of 10 μM gramicidin for 30 minutes at 37° C. and 340/380 nm fluorescence ratio determined.

FIGS. 4A-4D show that Bufalin inhibits IFNβ induction exclusively through the sodium pump. FIG. 4A, sequence alignment of the cardiac glycosides binding sites in human, mouse and rat ATP1a1 and ATP1a3 proteins. Q118R and N129D mutations in mouse and rat ATP1a1 make the rodent protein insensitive to cardiac glycosides treatment. FIG. 4B, mouse ATP1a1 gene fully rescued the inhibition of bufalin in human cells. 293T cells were transfected with various expression constructs together with IFNβ luciferase reporter. Cells were infected with SeV in the presence or absence of bufalin before measuring the luciferase activities. FIG. 4C, the catalytic activity of the ATP1a1 gene is required for the rescue. Experiments were conducted the same as in B, but a point mutation (D376E) of mouse ATP1a1 was tested together with the wild type expression construct. FIG. 4D, modulating intracellular ion concentration affects IFNb induction. 293T cells were transfected with IFNβ reporter construct, treated with various ion-channel ligands and then infected with SeV. Firefly luciferase activities were measured and normalized to renilla luciferase activities.

FIGS. 5A-5H show that knocking down sodium pump expression impairs IFNβ induction. FIG. 5A, efficient knock down of ATP1a1 expression in 293T cells. FIGS. 5B and 5C, SeV and dsDNA induced gene expression was impaired in ATP1a1 knock-down cells. Control or ATP1a1 knock-down cells were infected with SeV or transfected with dsDNA for 6 hrs. RNA was harvested and Q-PCR conducted to monitor the expression of IFNβ (B), Cxcl10 (C) genes. FIGS. 5D-5H, knocking-down ATP1a1 expression in MEFs also reduced virus, dsRNA and dsDNA induced gene expression. MEFs with shRNA targeting ATP1a1 or a scramble sequence as control were subjected to SeV, poly I:C and poly dA:dT treatment. 6 hrs later, cells were harvested for either protein analysis (FIG. 5D, blot for Stat1, Trex1, ATP1a1 and β-actin proteins) or Q-PCR analysis (FIGS. 5E-5H) for the expression of IFNβ (FIG. 5E); CXCL10 (FIG. 5F); IRF7 (FIG. 5G); and Stat1, Trex 1 and RIG-I (FIG. 5H) genes.

FIGS. 6A-6D show that Bufalin inhibits TNF signaling. FIG. 6A, Bufalin treatment reduced TNF induced NFκB activation in reporter assays. 293T cells were transfected with PRDII driving a luciferase reporter construct, treated with bufalin and TNF 24 hrs before measuring the luciferase activities. FIG. 6B, Bufalin inhibits TNF induced gene expression. 293T cells were treated with bufalin and TNF for 6 hrs, RNA extracted and subjected to RT-PCR analysis. FIG. 6C, Bufalin delays and decreases TNF induced NFκB activation. 293T cells were pretreated with bufalin for 30 min before addition of TNF to the medium, cells were harvested at indicated times and the protein level of IKBa determined by western blot analysis. FIG. 6D, Bufalin treatment interferes with nuclear translocation of p65. 293T cells with/without bufalin pretreatment were stimulated with TNF for 15 min, and then formaldehyde fixed and subjected to immunofluorescence staining with anti-p65 antibody.

FIGS. 7A-7E show Bufalin inhibits the induction of the IFNβ gene in Namalwa and Hela cells. FIG. 7A, structure of the Bufalin molecule. FIG. 7B, Bufalin blocks virus induced cytokines and ISG expression in Namalwa cells. Namalwa cells were grown in suspension and infected with Sendai virus (200 HAU/ml) in the presence or absence of bufalin (1 uM). 6 hrs later, cells were harvested and RNA extracted for RT-PCR analysis for the expression of various genes. FIG. 7C, Bufalin inhibited the dimerization of IRF3 in Namalwa cells. Namalwa cells were treated as in A, 6 hrs later, total protein extracts were prepared and subjected to native gel analysis. The formation of IRF3 dimer was monitored by probing the membrane with anti-IRF3 antibody. FIG. 7D, Bufalin inhibits IFNβ gene induction in Hela cells. Control or bufalin treated Hela cells were subjected to Sendai virus, dsRNA (poly I:C) or dsDNA (poly dA:dT) stimulation, 6 hrs later, cells were harvested and RNA extracted for RT-PCR analysis for the expression of IFNβ and GAPDH genes. FIG. 7E, expression profiles of the ATP1a1, RIG-I, MDA5, β-actin and HSP70 protein in 293T, Namalwa, Mg63, Hela and HT1080 human cell lines.

FIGS. 8A and 8B show that ouabain and digoxin potently inhibit virus induction of IFNβ expression. 293T cells were transfected with an IFNβ promoter driving luciferase reporter, 24 hrs later, increasing amounts (10 nM to 10 uM) of Ouabain (FIG. 8A) or digoxin (FIG. 8B) were added to cells before the Sendai virus infection. Luciferase activities were measured one day later. Cells were also treated with 1 uM bufalin as control.

FIG. 9 show that ion-channel modulators affect the IFNβ gene expression. 293T cells were treated with 10 uM of nimodipine, diazoxide or phenamil 30 min before Sendai virus infection, 6 hrs later total RNA were extracted and the expression of various genes were analyzed by RT-PCR.

FIGS. 10A and 10B show the effects of bufalin and ATP1a1 knockdown on the expression of IFNβ and ISGs in MEFs. FIG. 10A, wildtype MEFs were treated with bufalin (1 uM) before the infection with Sendai virus or transfection with dsRNA or dsDNA, 6 hrs later, total RNA were extracted and the expression of various genes were analyzed by RT-PCR. FIG. 10B, knocking down ATP1a1 expression in MEFs reduced the number of genes highly induced by various inducers. Total number of genes highly induced (from >1.5 fold to >3 fold) by virus, dsRNA, dsDNA in control and ATP1a1 knockdown MEFs were calculated from the microarray experiments.

FIG. 11 shows that RNA from bufalin and dsDNA double treated cells weakly induce IFNβ. 293T cells were treated with bufalin and infected with sendai virus, dsRNA (I:C) or dsDNA (dA:dT). Total RNA from these cells were extracted and re-transfected into fresh 293T cells (8 ug for 2 million cells) for 6 hrs, the induction of IFNβ and Cxcl10 genes from these samples were analyzed by RT-PCR. There was no difference for the induction of IFNβ by RNA from virus only or virus/bufalin double treated samples. RNA extracted from dsRNA treated samples was weakly induced most likely due to low levels of RNA inducer. RNA from dsDNA transfected cells strongly induced IFN and Cxcl10 expression, however this induction was eliminated by bufalin treatment.

FIGS. 12A and 12B show the effects of bufalin on the TNF induced activation of p65. FIG. 12A, 293T cells were pretreated with bufalin for 30 min, then stimulated with TNF (10 ng/ml) for the indicated times. Total protein eas extracted and analyzed by western blotting with antibodies against IKBa, phosphor-IKBa, phosphor-S276, phosphor-S468 and phosphor-S536 of the p65 protein, p65, traf6 and β-actin. While the initial degradation and phosphorylation of IKBa was the same between control and bufalin treated cells, the later phosphorylation and re-synthesis of IKBa, phosphorylation of S536 residue of p65 subunit is lower in bufalin treated samples. FIG. 12B, shows the effects of bufalin on TNF induced p65 nuclear translocation. 293T cells with/without bufalin pretreatment were stimulated with TNF for indicated time, and formaldehyde fixed and subjected to IF staining with anti-p65 antibody.

FIGS. 13A-13C show the dsRNA binding and ATPase activities of RIG-I. FIG. 13A, shows increasing RIG-1:dsRNA complex formation with increasing concentrations of RIG-I protein in binding assays. 200 ng of dsRNA was incubated with 0.5 μg and 1 μg of RIG-I protein at room temperature for 15 minutes and resolved on an agarose gel. FIG. 13B, shows that ATPase dead mutant K270A RIG-I protein displayed normal RNA binding activity. 0.5 μg of the recombinant mutant protein was incubated with 200 ng of dsRNA under different salt concentrations at room temperature for 15 minutes. Half of the samples was resolved in an agarose gel (top panel), and the other half was adjusted to 1 mM ATP, incubated for another 15 minutes at 37° C., and the released phosphates measured by Biomol Green reagents (bottom panel). The same amount of wild-type RIG-I protein was assayed for the ATPase activity as a control. FIG. 13C shows that bufalin does not directly inhibit the ATPase activity of RIG-I. The ATPase assay was conducted with RIG-I protein similarly as in FIG. 13B in the presence of increasing amounts of bufalin (200 nM and 1 μM). The released free phosphates were measured by Biomol Green reagents.

FIGS. 14A and 14B show that impaired IFNβ induction in ATP1a1 knock-down cells is not due to apoptosis. FIG. 14A, total protein lysates from 293T cells were separated on SDS-PAGE. Cells were either untreated, infected with lentivirus to specifically knock-down the expression of PARP1 or ATP1a1, or trearted with sturosporine (4 μM for 8 hours) to induce apoptosis. The expression of PARP1, cleaved PARAP1, cleaved Caspase3, ATP1a1 and beta-actin were analyzed by Western blot. FIG. 14B, shows that the induction of IFNβ and Cxcl10 was impaired in ATP1a1 knock-down cells. 293 T cells with PARP1 or ATP1a1 knocked-down were subjected to SeV infection or dsDNA (poly dA:dT) transfection. Total RNA was extracted after 6 hours and the expression of IFN-beta, Cxcl10, and GAPDH was analyzed by RT-PCR.

FIGS. 15A-15C show the effects of bufalin on IFN, LPS and EGF signaling. FIG. 15A shows that bufalin weakly inhibited IFN induced expression of Cxcl10 and RIG-I genes, but had no effect on the induction of Stat1 and ISG15 genes in Hela cells. FIG. 15B shows that bufalin only impaired LPS induced expression of Cxcl10 gene in THP-1 cells, while its effect Il-8, TNF and IKBa induction was minimal. FIG. 15C shows that bufalin had no effect on EGF induced phorpylation of MAP kinase P42/p44 in 293T cells. Cells were pretreated with or without bufalin (1 μM) for 30 minutes before treatment with the indicated stimulators (IFN, 1000 U/ml) for 6 hours; LPS, 20 ng/ml for 2 hours; EGF, 1 ug/ml for 22 hours). Total RNA or protein were harvested for the expression analysis of specific genes by RT-PCR or Western blot.

FIGS. 16A-16E show that bufalin does not induce apoptosis or autophagy in 293T cells. FIGS. 16A, 16B and 16E Show that bufalin does not severely impair cell viability in 293T cells. 293T cells were either untreated, or treated with increasing amounts of bufalin (1 nM to 10 μM) for 8 hours and subjected to either CellTiter-Blue viability assay (Promega) in which the fluorescence was recorded from 560 nm excitation/590 nm emission (FIG. 16A), or to CellTiter-Glo Luminescent viability assay (Promega) (FIG. 16B). The cells were also exposed to 0.1% Triton X-100 for 1 hr before the viability assay as a control (FIG. 16E). Data represent mean values±s.d. (n=3). FIG. 16C shows the flow cytometry analysis of 293T cells treated with different chemicals. 293T cells were treated with bufalin (1 μM for 8 hours), staurosporine (4 μM for 4 hours), DMSO or left untreated. Cells were harvested and washed, then stained with Allophycocyanin (APC) conjugated Annexin V and 7-AAD for 15 minutes and subjected to flow cytometry analysis. The number in each graph is the percentile of Annexin V or 7-AAD positive populations. FIG. 16D shows the Western blot analysis the analysis of apoptosis and autophagy. 293T cells were treated with increasing amounts of bufalin (1 nM to 10 μM) staurosporine (4 μM) or bafilomycin A1 (BFA, 100 nM) for 8 hours. Total protein lysates were prepared and separated on SDS-PAGE for Western blot analysis of PARP1, cleaved PARP1, cleaved Caspase3, LC3B, ATP1a1, and beta-actin expression. Bufalin treatment did not induce apoptosis or autophagy in 293T cells. Cleavage products of PARP1 and Caspase3 (an indication of apoptosis, induced by staurosporine), and the strong induction of LC3B-II (a marker of autophagy, induced by both staurosporine and BFA) were also not observed in bufalin treated cells.

FIG. 17 shows that bufalin treatment does not affect the efficiency of cell transfection. Cy3 labeled dsRNA (poly I:C) or dsDNA (poly dA:dT) were transfected into 293T cells pretreated with or without bufalin, medium was changed after 6 hours, cell images under Cy3 channel or direct phase contrast were taken 8 hours after transfection.

FIG. 18 is a schematic representation showing inhibition of virus induced IFNβ expression.

FIGS. 19A-19E show the full image of data shown in FIGS. 2B, 3A, 5A, 5D, and 6C respectively.

FIG. 20A shows the determination of a safe dosage for interperitoneal bufalin administration into ATP1a1 knock-in mice.

FIG. 20B shows that bufalin reduces the lethality induced by a high dose of LPS (80 mg/kg) in mice.

### DETAILED DESCRIPTION OF THE INVENTION

In one aspects the invention provides a method for inhibiting interferon-beta gene expression and/or reducing the level of interferon-beta in a cell, the method comprising contacting a cell with Na, Ca²⁺, or K⁺ ion-channel modulator.

The cell can be contacted with the ion-channel modulator in a cell culture e.g., in vitro or ex vivo, or the ion-channel modulator can be administrated to a subject, e.g., in vivo. In some embodiments of the invention, an ion-channel modulator can be administrated to a subject to treat, and/or prevent a disorder which is characterized by elevated levels of interferon-beta and/or elevated levels of interferon-beta gene expression.

The term “contacting” or “contact” as used herein in connection with contacting a cell includes subjecting the cell to an appropriate culture media which comprises the indicated ion-channel modulator. Where the cell is in vivo, “contacting” or “contact” includes administering the ion-channel modulator in a pharmaceutical composition to a subject via an appropriate administration route such that the ion-channel modulator contacts the cell in vivo.

For in vivo methods, a therapeutically effective amount of an ion-channel modulator can be administered to a subject. Methods of administering compounds to a subject are known in the art and easily available to one of skill in the art. Without wishing to be bound by theory, inhibition of interferon-beta gene expression and/or lowering of interferon-beta levels in a subject can lead to treatment, prevention or amelioration of a number of disorders which are characterized by elevated levels of interferon-beta gene expression and/or elevated levels of interferon-beta.

Without wishing to be bound by theory, interferon-beta gene expression can be measured by measuring the interferon-beta levels. Skilled artisan is well aware of the availability of commercial assays and kits for measuring interferon-beta. For example, Theremo Scientific sells interferon-beta ELISA kits for measuring human or mouse interferon-beta in cell culture supernatant. PBL InterferonSource (Piscataway, N.J., USA) sells the VeriKine-HS™ Human Interferon-Beta Serum ELISA kit (Cat. #41415-1) for measuring IFN-β in a variety of sample matrices including serum, plasma and tissue culture media. The interferon-beta gene expression can also be measured by measuring the in vivo activity of interferon-beta, e.g., by measuring IFN-stimulated genes (ISGs), such as Mx and PI-10 genes.

### Ion-Channel Modulators

As used herein, the term “ion-channel modulator” refers to a compound that modulates at least one activity of an ion-channel. The term “ion-channel modulator” as used herein is intended to include agents that interact with the channel pore itself, or that may act as an allosteric modulator of the channel by interacting with a site on the channel complex. The term “ion-channel modulator” as used herein is also intended to include agents that modulate activity of an ion-channel indirectly. By “indirectly,” as used in reference to modulator interactions with ion-channel, means the ion-channel modulator does not directly interact with the ion-channel itself, i.e., ion-channel modulator interacts with the ion-channel via an intermediary. Accordingly, the term “indirectly” also encompasses the situations wherein the ion-channel modulator requires another molecule in order to bind or interact with the ion-channel.

As used herein, the term “modulate” refers to a change or alternation in at least one biological activity of the ion-channel. Modulation may be an increase or a decrease in activity, a change in binding characteristics, or any other change in the biological, functional, or immunological properties of the ion-channel.

In some embodiments of the aspects described herein, the ion-channel modulator modulates the passage of ions through the ion-channel.

In some embodiments of the aspects described herein, the modulator is an inhibitor or antagonist of the ion-channel. As used herein, the term “inhibitor” refers to compounds which inhibit or decrease the flow of ions through an ion-channel.

In some embodiments of the aspects described herein, the modulator is an agonist of the ion-channel. As used herein, the term “agonist” refers to compounds which increase the flow of ions through an ion-channel.

In some embodiments of the aspects described herein, the modulator modulates at least one activity of the ion-channel by at least 5%, at least 10%, at least 15%, at least 20%, at least 25%, at least 30%, at least 40%, at least 50%, at least 60%, at least 70%, at least 80%, at least 90%, or at least 95%, at least 98% or more relative to a control with no modulation.

In some embodiments of the aspects described herein, at least one activity of the ion-channel is inhibited or lowered by at least 5%, at least 10%, at least 15%, at least 20%, at least 25%, at least 30%, at least 40%, at least 50%, at least 60%, at least 70%, at least 80%, at least 90%, at least 95%, at least 98%, or 100% (e.g. complete loss of activity) relative to control with no modulator.

In some embodiments of the aspects described herein, the ion-channel modulator has an IC₅₀, for inhibiting IFNβ expression, of less than or equal to 500 nM, 250 nM, 100 nM, 50 nM, 10 nM, 1 nM, 0.1 nM, 0.01 nM or 0.001 nM.

In some embodiments of the aspects described herein, the ion-channel modulator inhibits the flow of ions through the ion-channel by at least 5%, at least 10%, at least 15%, at least 20%, at least 25%, at least 30%, at least 40%, at least 50%, at least 60%, at least 70%, at least 80%, at least 90%, at least 95%, at least 98%, or 100% (e.g. complete stop of ion flow through the channel) relative to a control with no modulator.

In some embodiments of the aspects described herein, the ion-channel modulator increases the flow of ions through the ion-channel by at least 5%, at least 10%, at least 15%, at least 20%, at least 25%, at least 30%, at least 40%, at least 50%, at least 60%, at least 70%, at least 80%, at least 90%, at least 100%, at least 1.5 fold, at least by 2-fold, at least 3-fold, at least 4-fold, or at least 5-fold or more relative to a control with no modulator.

In some embodiments of the aspects described herein, the ion-channel modulator increases concentration of ions, e.g. sodium, in a cell by at least 5%, at least 10%, at least 15%, at least 20%, at least 25%, at least 30%, at least 40%, at least 50%, at least 60%, at least 70%, at least 80%, at least 90%, at least 100%, at least 1.5 fold, at least by 2-fold, at least 3-fold, at least 4-fold, or at least 5-fold or more relative to a control with no modulator.

Recently, Yang et al. reported that digitoxin, a cardiac glycoside, inhibits TNF signaling by blocking the recruitment of TRADD of the TNF receptor, and directly inhibiting IKBα degradation (Yang, et al., Cardiac glycosides inhibit TNF-alpha/NF-kappaB signaling by blocking recruitment of TNF receptor-associated death domin to the TNF receptor. Proc. Natl. Acad. Sci. USA 102, 9631-9636 (2005)). By contrast, the inventors demonstrated that IKBα degradation is not inhibited by bufalin. Accordingly, in some embodiments of the aspects described herein, the ion-channel modulator does not inhibit IKBα degradation, i.e. inhibits IKBα degradation by less than 50%, less than 25%, less than 20%, less than 15%, less than 10%, less than 5%, or less than 2.5% relative to non-inhibited control.

Without wishing to be bound by a theory, an ion-channel modulator can modulate the activity of an ion-channel through a number of different mechanisms. For example, a modulator can bind with the ion-channel and physically block the ions from going through the channel. An ion-channel modulator can bring about conformational changes in the ion-channel upon binding, which may increase or decrease the interaction between the ions and the channel or may increase or decrease channel opening.

A modulator can modulate the energy utilizing activity, e.g. ATPase activity, of the ion-channel. In some embodiments of the aspects described herein, the ion-channel modulator inhibits the ATPAse activity of the ion-channel.

In some embodiments of the aspects described herein, an ion-channel modulator inhibits ATPase activity of the Na⁺/K⁺-ATPase by at least 5%, at least 10%, at least 15%, at least 20%, at least 25%, at least 30%, at least 40%, at least 50%, at least 60%, at least 70%, at least 75%, at least 80%, at least 85%, at least 90%, at least 95%, at least 98%, or 100% (complete inhibition) relative to a control without the modulator. Without wishing to be bound by theory, ATPase activity can be measured by measuring the dephosphorylation of adenosine-triphosphate by utilizing methods well known to the skilled artisan for measuring such dephosphorylation reactions.

In some embodiments of the aspects described herein, an ion-channel modulator inhibits RIG-I activation by at least 5%, at least 10%, at least 15%, at least 20%, at least 25%, at least 30%, at least 40%, at least 50%, at least 60%, at least 70%, at least 75%, at least 80%, at least 85%, at least 90%, at least 95%, at least 98%, or 100% (complete inhibition) relative to a control without the modulator.

In some embodiments of the aspects described herein, an ion-channel modulator inhibits ATPase activity of RIG-I by at least 5%, at least 10%, at least 15%, at least 20%, at least 25%, at least 30%, at least 40%, at least 50%, at least 60%, at least 70%, at least 75%, at least 80%, at least 85%, at least 90%, at least 95%, at least 98%, or 100% (complete inhibition) relative to a control without the modulator.

Without limitation, the ion-channel modulator can be a small organic molecule, small inorganic molecule, a polysaccharide, a peptide, a protein, a nucleic acid, an extract made from biological materials such as bacteria, plants, fungi, animal cells, animal tissue, and any combinations thereof.

In some embodiments, the modulator is an antiarrhythmic agent. As used herein, the term “antiarrhythmic agent” refers to compounds that are used to treat, or control, cardiac arrhythmias, such as atrial fibrillation, atrial flutter, ventricular tachycardia, and ventricular fibrillation. Generally an antiarrhythmic agent's mechanism of action conforms to one or more of the four Vaughan-Williams classifications. The four main classes in the Vaughan Williams classification of antiarrhythmic agents are as follow: Class I agents interfere with the Na⁺ channel; Class II agents are anti-sympathetic nervous system agents, most agents in this class are beta blockers; Class III agents affect K⁺ efflux; and Class IV agents affect Ca²⁺ channels and the AV node. Since the development of the original Vaughan-Williams classification system, additional agents have been used that don't fit cleanly into categories I through IV. These agents are also included in the term “antiarrhythmic agent.”

Exemplary antiarrhythmic agents include, but are not limited to, Quinidine, Procainamide, Disopyramide, Lidocaine, Phenyloin, Flecamide, Propafenone, Moricizine, Propranolol, Esmolol, Timolol, Metoprolol, Atenolol, Bisoprolol, Amiodarone, Sotalol, Ibutilide, Dofetilide, E-4031, Diltiazem, Adenosine, Digoxin, adenosine, magnesium sulfate, and analogs, derivatives, pharmaceutically acceptable salts, and/or prodrugs thereof.

In some embodiments of the aspects described herein, the ion-channel modulator is a cardiac glycoside. As used herein, the term “cardiac glycoside” refers to the category of compounds that have a positive inotropic effects on the heart. Cardiac glycosides are also referred to as cardiac steroids in the art. They are used in treatment of heart diseases, including cardiac arrhythmia and have a rate dependent effect upon AV nodal conduction. As a general class of compounds, cardiac glycosides comprise a steroid core with either a pyrone or butenolide substituent at C17 (the “pyrone form” and “butenolide form”). Additionally, cardiac glycosides may optionally be glycosylated at C3. The form of cardiac glycosides without glycosylation is also known as “aglycone.” Most cardiac glycosides include one to four sugars attached to the 3β-OH group. The sugars most commonly used include L-rhamnose, D-glucose, D-digitoxose, D-digitalose, D-digginose, D-sarmentose, L-vallarose, and D-fructose. In general, the sugars affect the pharmacokinetics of a cardiac glycoside with little other effect on biological activity. For this reason, aglycone forms of cardiac glycosides are available and are intended to be encompassed by the term “cardiac glycoside” as used herein. The pharmacokinetics of a cardiac glycoside may be adjusted by adjusting the hydrophobicity of the molecule, with increasing hydrophobicity tending to result in greater absorption and an increased half-life. Sugar moieties may be modified with one or more groups, such as an acetyl group.

A large number of cardiac glycosides are known in the art. Exemplary cardiac glycoside include, but are not limited to, bufalin, ouabain, digitoxigenin, digoxin, lanatoside C, Strophantin K, uzarigenin, desacetyllanatoside A, digitoxin, actyl digitoxin, desacetyllanatoside C, strophanthoside, scillarenin, scillaren A, proscillaridin, proscillaridin A, BNC-1, BNC-4, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, strophanthidine, strophanthidine digilanobioside, strophanthidin-Dcymaroside, digoxigenin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin, calotoxin, lanatoside A, uzarin, strophanthidine-3β-digitoxoside, strophanthidin a-L-rhamnopyranoside, and analogs, derivatives, pharmaceutically acceptable salts, and/or prodrugs thereof.

More than a hundred cardiac glycosides have been identified as secondary metabolites in plants, with most belonging to the angiosperms. See for example, Melero, C. P., Medardea, M. & Feliciano, A. S. A short review on cardiotonic steroids and their aminoguanidine analogues. Molecules 5, 51-81 (2000), content of which is herein incorporated by reference. Generally, cardiac glycosides are found in a diverse group of plants including Digitalis purpurea and Digitalis lanata (foxgloves), Nerium oleander (common oleander), Thevetia peruviana (yellow oleander), Convallaria majalis (lily of the valley), Urginea maritima and Urginea indica (squill), and Strophanthus gratus (ouabain). Recently, however, cardiac glycosides of the bufadienolide class were identified in the skin and the carotid gland of animals, and mainly in the venom of several toad species. See Steyn, P. S. & van Heerden, F. R. Bufadienolides of plant and animal origin. Nat. Prod. Rep. 15, 397-413 (1998), content of which is herein incorporated by reference.

In some embodiments of the aspects described herein, the ion-channel modulator is a sodium pump blocker. As used herein, the terms “sodium pump blocker,” “sodium pump inhibitor,” and “sodium pump antagonist” refer to compounds that inhibit or block the flow of sodium and/or potassium ions across a cell membrane.

In some embodiments of the aspects described herein, the ion-channel modulator is a calcium channel blocker. As used herein, the terms “calcium channel blocker,” “calcium channel inhibitor,” and “calcium channel antagonist” refer to compounds that inhibit or block the flow of calcium ions across a cell membrane. Calcium channel blockers are also known as calcium ion influx inhibitors, slow channel blockers, calcium ion antagonists, calcium channel antagonist drugs and as class IV antiarrhythmics. Exemplary calcium channel blocker include, but are not limited to, amiloride, amlodipine, bepridil, diltiazem, felodipine, isradipine, mibefradil, nicardipine, nifedipine (dihydropyridines), nickel, nimodinpine, nisoldipine, nitric oxide (NO), norverapamil, verapamil, and analogs, derivatives, pharmaceutically acceptable salts, and/or prodrugs thereof.

In some embodiments of the aspects described herein, the calcium channel blocker is a beta-blocker. Exemplary beta-blockers include, but are not limited to, Alprenolol, Bucindolol, Carteolol, Carvedilol (has additional α-blocking activity), Labetalol, Nadolol, Penbutolol, Pindolol, Propranolol, Timolol, Acebutolol, Atenolol, Betaxolol, Bisoprolol, Celiprolol, Esmolol, Metoprolol, Nebivolol, Butaxamine, and ICI-118,551 (3-(isopropylamino)-1-[(7-methyl-4-indanyl)oxy]butan-2-ol), and analogs, derivatives, pharmaceutically acceptable salts, and/or prodrugs thereof.

Exemplary K⁺ ion-channel modulators include, but are not limited to, 2,3-Butanedione monoxime; 3-Benzidino-6-(4-chlorophenyl)pyridazine; 4-Aminopyridine; 5-(4-Phenoxybutoxy)psoralen; 5-Hydroxydecanoic acid sodium salt; L-α-Phosphatidyl-D-myo-inositol; 4,5-diphosphate, dioctanoyl; Aa1; Adenosine 5′-(β,γ-imido)triphosphate tetralithium salt hydrate; Agitoxin-1; Agitoxin-2; Agitoxin-3; Alinidine; Apamin; Aprindine hydrochloride; BDS-I; BDS-II; BL-1249; BeKm-1; CP-339818; Charybdotoxin; Charybdotoxin; Chlorzoxazone; Chromanol 293B; Cibenzoline succinate; Clofilium tosylate; Clotrimazole; Cromakalim; CyPPA; DK-AH 269; Dendrotoxin-I; Dendrotoxin-K; Dequalinium chloride hydrate; DPO-1 needles; Diazoxide; Dofetilide; E-4031; Ergtoxin; Glimepiride; Glipizide; Glybenclamide; Heteropodatoxin-2; Hongotoxin-1; ICA-105574; IMID-4F hydrochloride; Iberiotoxin; Ibutilide hemifumarate salt; Isopimaric Acid; Kaliotoxin-1; Levcromakalim; Lq2; Margatoxin; Mast Cell Degranulating Peptide; Maurotoxin; Mephetyl tetrazole; Mepivacaine hydrochloride; Minoxidil; Minoxidil sulfate salt; N-Acetylprocainamide hydrochloride; N-Salicyloyltryptamine; NS 1619; NS1643; NS309; NS8593 hydrochloride; Nicorandil; Noxiustoxin; Omeprazole; PD-118057; PNU-37883A; Pandinotoxin-Kα; Paxilline; Penitrem A; Phrixotoxin-2; Pinacidil monohydrate; Psora-4; Quinine; Quinine hemisulfate salt monohydrate; Quinine hydrobromide; Quinine hydrochloride dehydrate; Repaglinide; Rutaecarpine; S(+)-Niguldipine hydrochloride; SG-209; Scyllatoxin; Sematilide monohydrochloride monohydrate; Slotoxin; Stromatoxin-1; TRAM-34; Tamapin; Tertiapin; Tertiapin-Q trifluoroacetate salt; Tetracaine; Tetracaine hydrochloride; Tetraethylammonium chloride; Tityustoxin-Kα; Tolazamide; UCL 1684; UCL-1848 trifluoroacetate salt; UK-78282 monohydrochloride; VU 590 dihydrochloride hydrate; XE-991; ZD7288 hydrate; Zatebradine hydrochloride; α-Dendrotoxin; β-Dendrotoxin; δ-Dendrotoxin; γ-Dendrotoxin; β-Bungarotoxin; and analogs, derivatives, pharmaceutically acceptable salts, and/or prodrugs thereof.

In some embodiments of the aspects described herein, the ion-channel modulator is a potassium channel agonist. As used herein, a “potassium channel agonist” is a K⁺ ion-channel modulator which facilitates ion transmission through K⁺ ion-channels. Exemplary potassium channel agonists include, but are not limited to diazoxide, minoxidil, nicorandil, pinacidil, retigabine, flupirtine, lemakalim, L-735534, and analogs, derivatives, pharmaceutically acceptable salts, and/or prodrugs thereof.

In some embodiments of the aspects described herein, the ion-channel modulator is selected from the group consisting of bufalin; digoxin; ouabain; nimodipine; diazoxide; digitoxigenin; ranolazine; lanatoside C; Strophantin K; uzarigenin; desacetyllanatoside A; actyl digitoxin; desacetyllanatoside C; strophanthoside; scillaren A; proscillaridin A; digitoxose; gitoxin; strophanthidiol; oleandrin; acovenoside A; strophanthidine digilanobioside; strophanthidin-d-cymaroside; digitoxigenin-L-rhamnoside; digitoxigenin theretoside; strophanthidin; digoxigenin-3,12-diacetate; gitoxigenin; gitoxigenin 3-acetate; gitoxigenin-3,16-diacetate; 16-acetyl gitoxigenin; acetyl strophanthidin; ouabagenin; 3-epigoxigenin; neriifolin; acetyhieriifolin cerberin; theventin; somalin; odoroside; honghelin; desacetyl digilanide; calotropin; calotoxin; convallatoxin; oleandrigenin; periplocyrnarin; strophanthidin oxime; strophanthidin semicarbazone; strophanthidinic acid lactone acetate; ernicyrnarin; sannentoside D; sarverogenin; sannentoside A; sarmentogenin; proscillariditi; marinobufagenin; Amiodarone; Dofetilide; Sotalol; Ibutilide; Azimilide; Bretylium; Clofilium; N-[4-[[1-[2-(6-Methyl-2-pyridinyl)ethyl]-4-piperidinyl]carbonyl]phenyl]methanesulfonamide (E-4031); Nifekalant; Tedisamil; Sematilide; Ampyra; apamin; charybdotoxin; 1-Ethyl-2-benzimidazolinone (1-EBIO); 3-Oxime-6,7-dichloro-1H-indole-2,3-dione (NS309); Cyclohexyl-[2-(3,5-dimethyl-pyrazol-1-yl)-6-methyl-pyrimidin-4-yl]-amine (CyPPA); GPCR antagonists; ifenprodil; glibenclamide; tolbutamide; diazoxide; pinacidil; halothane; tetraethylammonium; 4-aminopyridine; dendrotoxins; retigabine; 4-aminopyridine; 3,4-diaminopyridine; diazoxide; Minoxidil; Nicorandi; Retigabine; Flupirtine; Quinidine; Procainamide; Disopyramide; Lidocaine; Phenyloin; Mexiletine; Flecamide; Propafenone; Moricizine; atenolol; ropranolol; Esmolol; Timolol; Metoprolol; Atenolol; Bisoprolol; Amiodarone; Sotalol; Ibutilide; Dofetilide; Adenosine; Nifedipine; δ-conotoxin; κ-conotoxin; μ-conotoxin; ω-conotoxin; ω-conotoxin GVIA; ω-conotoxin ω-conotoxin CNVIIA; ω-conotoxin CVIID; ω-conotoxin AM336; cilnidipine; L-cysteine derivative 2A; ω-agatoxin WA; N,N-dialkyl-dipeptidyl-amines; SNX-111 (Ziconotide); caffeine; lamotrigine; 202W92 (a structural analog of lamotrigine); phenyloin; carbamazepine; 1,4-dihydro-2,6-dimethyl-5-nitro-4-[thieno[3,2-c]-pyridin-3-yl]-3-pyridinecarboxylic acid, 1-phenylethyl ester; 1,4-dihydro-2,6-dimethyl-5-nitro-4-[thieno[3,2-c]-pyridin-3-yl]-3-pyridinecarboxylic acid, 1-methyl-2-propynyl ester; 1,4-dihydro-2,6-dimethyl-5-nitro-4-[3,2-c]pyridin-3-yl]-3-pyridinecarboxylic acid, cyclopropylmethyl ester; 1,4-dihydro-2,6-dimethyl-5-nitro-4-[thieno(3,2-c)pyridin-3-yl]-3-pyridinecarboxylic acid, butyl ester; (S)-1,4-Dihydro-2,6-dimethyl-5-nitro-4-[thieno[3,2c]pyridin-3-yl]-3-pyridinecarboxylic acid, 1-methylpropyl ester; 1,4-Dihydro-2,6-dimethyl-5-nitro-4-thieno[3,2-c]pyridin-3-yl]-3-pyridinecarboxylic acid, methyl ester; 1,4-Dihydro-2,6-dimethyl-5-nitro-4-[thieno[3,2-c]pyridin-3-yl]-3-pyridinecarboxylic acid, 1-methylethyl ester; 1,4-Dihydro-2,6-dimethyl-5-nitro-4-thieno[3,2-c]pyridin-3-yl]-3-pyridinecarboxylic acid, 2-propynyl ester; 1,4-Dihydro-2,6-dimethyl-5-nitro-4-[thieno[3,2-c]pyridin-3-yl]-3-pyridinecarboxylic acid, 1-methyl-2propynyl ester; 1,4-Dihydro-2,6-dimethyl-5-nitro-4-[thieno[3,2-c]pyridin-3-yl]-3-pyridinecarboxylic acid, 2-butynyl este; 1,4-Dihydro-2,6-dimethyl-5-nitro-4-[thieno[3,2-c]pyridin-3-yl]-3-pyridinecarboxylic acid, 1-methyl-2butynyl este; 1,4-Dihydro-2,6-dimethyl-5-nitro-4-[thieno[3,2-c]pyridin-3-yl]-3-pyridinecarboxylic acid, 2,2-dimethylpropyl ester; 1,4-Dihydro-2,6-dimethyl-5-nitro-4-thieno[3,2-c]pyridin-3-yl]-3-pyridinecarboxylic acid, 3-butynyl ester; 1,4-Dihydro-2,6-dimethyl-5-nitro-4-[thieno3,2-c]pyridin-3-yl]-3-pyridinecarboxylic acid, 1,1-dimethyl-2propynyl ester; 1,4-Dihydro-2,6-dimethyl-5-nitro-4-[thieno-3,2-c]pyridin-3-yl-3-pyridinecarboxylic acid, 1,2,2-trimethylpropyl ester; R(+)-1,4-Dihydro-2,6-dimethyl-5-nitro-4[thieno[3,2-c]pyridin-3-yl]-3-pyridinecarboxylic (2Amethyl-1-phenylpropyl) ester; S-(−)-1,4-Dihydro-2,6-dimethyl-5-nitro-4[thieno[3,2-c]pyridin-3-yl]-3-pyridinecarboxylic acid, 2-methyl-1-phenylpropyl ester; 1,4-Dihydro-2,6-dimethyl-5-nitro-4-[thieno[3,2c]-pyridin-3-yl]-3-pyridinecarboxylic acid, 1-methylphenylethyl ester; 1,4-Dihydro-2,6-dimethyl-5-nitro-4-[thieno[3,2-c]pyridin-3-yl]-3-pyridinecarboxylic acid, 1-phenylethyl ester; 1,4-Dihydro-2,6-dimethyl-5-nitro-4-[thieno[3,2c]-pyridin-3-yl]-3-pyridinecarboxylic acid, (1-phenylpropyl)ester; 1,4-Dihydro-2,6-dimethyl-5-nitro-4-[thieno[3,2c]-pyridin-3-yl]-3-pyridinecarboxylic acid, (4-methoxyphenyl)methyl ester; 1,4-Dihydro-2,6-dimethyl-5-nitro-4-[thieno[3,2c]-pyridin-3-yl]-3-pyridinecarboxylic acid, 1-methyl-2-phenylethyl ester; 1,4-Dihydro-2,6-dimethyl-5-nitro-4-[thieno[3,2c]-pyridin-3-yl]-3-pyridinecarboxylic acid, 2-phenylpropyl ester; 1,4-Dihydro-2,6-dimethyl-5-nitro-4-[thieno[3,2c]-pyridin-3-yl]-3-pyridinecarboxylic acid, phenylmethyl ester; 1,4-Dihydro-2,6-dimethyl-5-nitro-4-[thieno[3,2c]-pyridin-3-yl]-3-pyridinecarboxylic acid, 2-phenoxyethyl ester; 1,4-Dihydro-2,6-dimethyl-5-nitro-4-thieno-3,2-c]pyridin-3-yl]-3-pyridinecarboxylic acid, 3-phenyl-2propynyl este; 1,4-Dihydro-2,6-dimethyl-5-nitro-4-[thieno[3,2c]-pyridin-3-yl]-3-pyridinecarboxylic acid, 2-methoxy-2-phenylethyl ester; (S)-1,4-Dihydro-2,6-dimethyl-5-nitro-4-[thieno[3,2-c]pyridin-3-yl]-3-pyridinecarboxylic acid, 1-phenylethyl este; (R)-1,4-Dihydro-2,6-dimethyl-5-nitro-4-[thieno[3,2-c]pyridin-3-yl]-3-pyridinecarboxylic acid, 1-phenylethyl este; 1,4-Dihydro-2,6-dimethyl-5-nitro-4-[thieno[3,2c]-pyridin-3-yl]-3-pyridinecarboxylic acid, cyclopropylmethyl ester; 1,4-Dihydro-2,6-dimethyl-5-nitro-4-thieno[3,2-c]pyridin-3-yl]-3-pyridinecarboxylic acid, 1-cyclopropylethyl este; 1,4-Dihydro-2,6-dimethyl-5-nitro-4-[thieno[3,2c]-pyridin-3-yl]-3-pyridinecarboxylic acid, 2-cyanoethyl ester; 1,4-Dihydro-4-(2-{5-[4-(2-methoxyphenyl)-1-1piperazinyl]pentyl}-3-furanyl)-2,6-dimethyl-5-nitro-3-pyridinecarboxylic acid, methyl ester; 4-(4-Benzofurazanyl)-1,4-dihydro-2,6-dimethyl-5-nitro-3-pyridinecarboxylic acid, {4-[4-(2-methoxyphenyl)-1-piperazinyl]butyl}ester; 1,4-Dihydro-2,6-dimethyl-5-nitro-4-(3-pyridinyl)-3-pyridinecarboxylic acid, {4-[4-(2-pyrimidinyl)-1-piperazinyl]butyl}ester; 4-(3-Furanyl)-1,4-dihydro-2,6-dimethyl-5-nitro-3pyridinecarboxylic acid, {2-[4-(2-methoxyphenyl)-1piperazinyl]ethyl}ester; 4-(3-Furanyl)-1,4-dihydro-2,6-dimethyl-5-nitro-3pyridinecarboxylic acid, {2-[4-(2-pyrimidinyl)-1piperazinyl]ethyl}ester; 1,4-Dihydro-2,6-dimethyl-4-(1-methyl-1H-pyrrol-2-yl)-5-nitro-3-pyridinecarboxylic acid, {4-[4-(2-methoxyphenyl)1-piperazinyl]butyl}ester; 1,4-Dihydro-2,6-dimethyl-4-(1-methyl-1H-pyrrol-2-yl)-5-nitro-3-pyridinecarboxylic acid, {4-[4-(2pyrimidinyl)-1-piperazinyl]butyl}ester; 1,4-Dihydro-2,6-dimethyl-5-nitro-4-(3-thienyl)-3-pyridinecarboxylic acid, {2-[4-(2-methoxyphenyl)-1-piperazinyl]ethyl}ester; 1,4-Dihydro-2,6-dimethyl-5-nitro-4-(3-thienyl)-3-pyridinecarboxylic acid, {2-[4-(2-pyrimidinyl)-1-piperazinyl]ethyl}ester; 4-(3-Furanyl)-1,4-dihydro-2,6-dimethyl-5-nitro-3-pyridinecarboxylic acid, {4-[4-(2-pyrimidinyl)-1-piperazinyl]butyl}ester; (4-(2-Furanyl)-1,4-dihydro-2,6-dimethyl-5-nitro-3-pyridinecarboxylic acid, {4-[4-(2-pyrimidinyl)-1-piperazinyl]butyl}ester; 1,4-Dihydro-2,6-dimethyl-5-nitro-4-(2-thienyl)-3-pyridinecarboxylic acid, {2-[4-(2-methoxyphenyl)-1-piperazinyl]ethyl}ester; 1,4-Dihydro-2,6-dimethyl-4-(1-methyl-1H-pyrrol-2-yl)-5-nitro-3-pyridinecarboxylic acid, {2-[4-(2-methoxyphenyl)-1-piperazinyl]ethyl}ester; 1,4-Dihydro-2,6-dimethyl-4-(1-methyl-1H-pyrrol-2-yl)-5-nitro-3-pyridinecarboxylic acid, {2-[4-(2pyrimidinyl)1-piperazinyl]ethyl}ester; 5-(4-Chlorophenyl)-N-(3,5-dimethoxyphenyl)-2-furancarboxamide (A-803467); and analogs, derivatives, pharmaceutically acceptable salts, and/or prodrugs thereof.

In some embodiments of the aspects described herein, the modulator is not an amiloride or analog or derivative thereof. Accordingly, in some embodiments of the aspects described herein, the ion-channel modulator is not phenamil.

In some embodiments of the aspects described herein, the ion-channel modulator is bufalin or analogs, derivatives, pharmaceutically acceptable salts, and/or prodrugs thereof. Exempalry bufalin analogs and derivatives include, but are not limited to, 7β-Hydroxyl bufalin; 3-epi-7β-Hydroxyl bufalin; 1β-Hydroxyl bufalin; 15α-Hydroxyl bufalin; 15β-Hydroxyl bufalin; Telocinobufagin (5-hydroxyl bufalin); 3-epi-Telocinobufagin; 3-epi-Bufalin-3-O-β-d-glucoside; 11β-Hydroxyl bufalin; 12β-Hydroxyl bufalin; 1β,7β-Dihydroxyl bufalin; 16α-Hydroxyl bufalin; 7β,16α-Dihydroxyl bufalin; 1β,12β-Dihydroxyl bufalin; resibufogenin; norbufalin; 3-hydroxy-14(15)-en-19-norbufalin-20,22-dienolide; 14-dehydrobufalin; bufotalin; arenobufagin; cinobufagin; marinobufagenin; proscillaridin; scillroside; scillarenin; and 14,15-epoxy-bufalin. Without limitation, analogs and derivatives of bufalin include those that can cross the blood-brain barrier. Herein, bufadienolides and analogs and derivatives thereof are also considered bufalin analaogs or derivatives thereof. Further bufalin or bufadienolide analogs and derivatives amenable to the present invention include those described in U.S. Pat. No. 3,080,362; No. 3,136,753; No. 3,470,240; No. 3,560,487; No. 3,585,187; No. 3,639,392; No. 3,642,770; No. 3,661,941; No. 3,682,891; No. 3,682,895; No. 3,687,944; No. 3,706,727; No. 3,726,857; No. 3,732,203; No. 3,806,502; No. 3,812,106; No. 3,838,146; No. 4,001,401; No. 4,102,884; No. 4,175,078; No. 4,242,33; No. 4,380,624; No. 5,314,932; No. 5,874,423; and No. 7,087,590 and those described in Min, et al., J. Steroid. Biochem. Mol. Biol., 91(1-2): 87-98 (2004); Kamano, Y. & Pettit, G. R. J. Org. Chem., 38 (12): 2202-2204 (1973); Watabe, et al., Cell Growth Differ, 8(8): 871 (1997); and Mahringer et al., Cancer Genomics and Proteomics, 7(4): 191-205 (2010). Content of all of patents and references listed in this paragraph is herein incorporated by reference.

A wide variety of entities (ligands) can be coupled to bufadienolide or analogs or derivatives thereof. For example, a ligand can be attached to the hydroxyl at the 3 position of bufadienolide analogs and derivatives which comprise a hydroxyl at the 3 position. When the a ligand is present at the 3 hydroxyl position, the ligand can be in the α or β configuration relative to the sterol ring system. A ligand can alter the distribution, targeting or lifetime of the molecule with which it is linked. In some embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Ligands providing enhanced affinity for a selected target are also termed targeting ligands. Ligands in general can include therapeutic modifiers, e.g., for enhancing uptake; diagnostic compounds; or reporter groups e.g., for monitoring distribution. General examples include lipids, steroids, vitamins, sugars, proteins, peptides, polyamines, peptide mimics, and oligonucleotides.

Ligands can include a naturally occurring substance, such as a protein (e.g., human serum albumin (HSA), low-density lipoprotein (LDL), high-density lipoprotein (HDL), or globulin); a carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Other examples of ligands include dyes, porphyrins (TPPC4, texaphyrin, Sapphyrin), polycyclic aromatic hydrocarbons (e.g., phenazine, dihydrophenazine), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidyl species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers. The ligand can be, for example, a lipopolysaccharide, an activator of p38 MAP kinase, or an activator of NF-κB.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HAS, low density lipoprotein (LDL) and high-density lipoprotein (HDL).

In some preferred embodiments, the ligand is a carbohydrate, e.g., monosaccharide, disaccharide, trisaccharide, oligosaccharide, and polysaccharide. Exemplary carbohydrate ligands include, but are not limited to, ribose, arabinose, xylose, lyxose, ribulose, xylulose, allose, altrose, glucose, mannose, gulose, idose, galactose, N—Ac-galatose, talose, psicose, fructose, sorbose, tagatose, fucose, fuculose, rhamonse, sedoheptulose, octose, nonose (neuraminic acid), sucrose, lactose, maltose, trehalose, turanose, cellobiose, raffinose, melezitose, maltotriose, acarbose, stachyose, fructooligosaccharide, galactooligosaccharides, mannanoligosaccharides, glycogen, starch (amylase, amylopectin), cellulose, beta-glucan (zymosan, lentinan, sizofiran), maltodextrin, inulin, levan beta (2->6), chitin, wherein the carbohydrate may be optionally substituted.

When the carbohydrate ligand comprises two or more sugars, each sugar can be independently selected from the group consisting of ribose, arabinose, xylose, lyxose, ribulose, xylulose, allose, altrose, glucose, mannose, gulose, idose, galactose, N—Ac-galatose, talose, psicose, fructose, sorbose, tagatose, fucose, fuculose, rhamonse, sedoheptulose, octose, and nonose (neuraminic acid), wherein the sugar may be optionally substituted. Without limitation each sugar can independently have the L- or the D-conformation. Furthermore, the linkage between two sugars can be independently α or β.

### Ion-Channels

As use herein, the term “ion-channel” refers to a transmembrane pore that presents a hydrophilic channel for specific ions to cross a lipid bilayer down their electrochemical gradients. There are over 300 types of ion-channels in a living cell (Gabashvili, et al., “Ion-channel gene expression in the inner ear”, J. Assoc. Res. Otolaryngol. 8 (3): 305-28 (2007), content of which is herein incorporated by reference). The ion-channels are classified upon their ion specificity, biological function, regulation or molecular structure, and nature of their gating. Examples of ion-channels are voltage gated ion-channels, Gap-junction ion-channels, ligand-gated ion-channels, ATP-gated ion-channels, heat-activated ion-channels, intracellular ion-channels, ion-channels gated by intracellular ligands such as cyclic nucleotide-gated channels or calcium-activated ion-channels. As used herein the term “gated ion-channel” is defined as an ion-channel the passage of ions through which is dependent on the presence of an analyte.

Ion-channels can be either anion-channels or cation-channels. Anion-channels are channels that facilitate the transport of anions across cell membranes. The anions being transported include, for example, chloride, bicarbonate, and organic ions such as bile acids. Cation-channels, on the other hand, are channels that facilitate the transport of cations across cell membranes. The cations being transported may be divalent cations such as Ca⁺² or Ba⁺² or monovalent cations such as Na⁺, K⁺ or H⁺. By facilitating transport and/or diffusion, ion-channels enable particular anions or cations to cross the cell membrane at a greater rate than would normally occur based on simple diffusion through the membrane. While not intending to be bound by theory, it is believed that ion-channels contain a receptor site within their pore structure that is specific for the anion(s) or cation(s) that they transport, and that binding of an ion or ions to the receptor site results in a conformation change that allows the bound ion to pass through the membrane, resulting in either passage either into or out of the cell. Ion-channels are also referred to as ion transporters.

In some embodiments of the aspects described herein, the ion-channel is a Na⁺, Ca²⁺ or K⁺ ion-channel.

As used herein, a “Na⁺ ion-channel” is an ion-channel which displays selective permeability to Na⁺ ions.

As used herein, a “Ca²⁺ ion-channel” is an ion-channel which displays selective permeability to Ca²⁺ ions. It is sometimes synonymous as voltage-dependent calcium channel, although there are also ligand-gated calcium channels. See for example, F. Striggow and B. E. Ehrlich, “Ligand-gated calcium channels inside and out”, Curr. Opin. Cell Biol. 8 (4): 490-5 (1996). Exemplary Ca²⁺ ion-channels include, but are not limited to, L-type, P-type/Q-type, N-type, R-type, and T-type. In some embodiments of the aspects described herein, the Ca²⁺ ion-channel is a L-type Ca²⁺ ion-channel.

As used herein, a “K⁺ ion-channel” is an ion-channel which displays selective permeability to K⁺ ions. There are four major classes of potassium channels: calcium-activated potassium channel, which opens in response to presence of calcium ions or other signaling molecules; inwardly rectifying potassium channel, which passes current (positive charge) more easily in the inward direction (into the cell); tandem pore domain potassium channels, which are constitutively open or possess high basal activation; and voltage-gated potassium channels, which open or close in response to changes in the transmembrane voltage.

Exemplary K⁺ ion-channel include, but are not limited to, BK channel, SK channel, ROMK (Kᵢᵣ1.1), GPCR regulated (Kᵢᵣ3.x), ATP-sensitive (Kᵢᵣ6.x), TWIK, TRAAK, TREK, TASK, hERG (Kᵥ 11.1), and KvLQT1 (Kᵥ 7.1). In some embodiments of the aspects described herein, the K⁺ ion-channel is a ATP-sensitive K⁺ channel. As used herein, an “ATP-sensitive channel” is a K⁺ ion-channel that is that is gated by ATP. ATP-sensitive potassium channels are composed of Kᵢᵣ6.x-type subunits and sulfonylurea receptor (SUR) subunits, along with additional components. See for example, Stephan, et al., “Selectivity of repaglinide and glibenclamide for the pancreatic over the cardiovascular K(ATP) channels”, Diabetologia 49 (9): 2039-48 (2006), content of which is herein incorporated by reference in its entirety. ATP-sensitive K⁺ channels can be further identified by their position within the cell as being either sarcolemmal (“sarcK_{ATP}”), mitochondrial (“mitoK_{ATP}”), or nuclear (“nucK_{ATP}”).

In some embodiments of the aspects described herein, the ion-channel is a Na⁺/K⁺ pump. The Na⁺/K⁺ pump is also referred to as simply as the sodium pump in the art. The Na⁺/K⁺ pump is an electrogeneic transmembrane ATPase. It is a highly-conserved integral membrane protein that is expressed in virtually all cells of higher organisms. The sodium pump is responsible for the maintenance of ionic concentration gradients across the cell membrane by pumping three Na⁺ out of the cell and two K⁺ into the cell. Since this channel requires the expenditure of energy by hydrolysis of ATP for this action, it is, therefore, called as Na⁺/K⁺-ATPase. It has been estimated that roughly 25% of all cytoplasmic ATP is hydrolyzed by sodium pumps in resting humans. In nerve cells, approximately 70% of the ATP is consumed to drive Na⁺/K⁺-ATPase. The Na⁺/K⁺-ATPase helps maintain resting potential, avail transport, and regulate cellular volume. It also functions as signal transducer/integrator to regulate MAPK pathway, ROS, as well as intracellular calcium.

One of the important functions of Na⁺/K⁺ pump is to maintain the volume of the cell. Inside the cell there are many proteins and other organic compounds that cannot escape from the cell. Most, being negatively charged, collect around them a large number of positive ions. All these substances tend to cause the osmosis of water into the cell, which, unless checked, can cause the cell to swell up and lyse. The Na⁺/K⁺ pump is a mechanism to prevent this. The pump transports 3 Na⁺ ions out of the cell and in exchange takes 2 K⁺ ions into the cell. As the membrane is far less permeable to Na⁺ ions than K⁺ ions the sodium ions have a tendency to stay there. This represents a continual net loss of ions out of the cell. The opposing osmotic tendency that results operates to drive the water molecules out of the cells. Furthermore, when the cell begins to swell, this automatically activates the Na⁺—K⁺ pump, which moves still more ions to the exterior.

In addition to pumping ions, it is now established that Na⁺/K⁺-ATPase acts as a scaffold for the assembly of a multiple-protein signalling domain that transmits signals to various intracellular compartments. See for example, Haas, et al., Src-mediated inter-receptor cross-talk between the Na⁺/K⁺-ATPase and the epidermal growth factor receptor relays the signal from ouabain to mitogen-activated protein kinases. J. Biol. Chem. 277, 18694-18702 (2002); Haas, et al., Involvement of Src and epidermal growth factor receptor in the signal-transducing function of Na⁺/K⁺-ATPase. J. Biol. Chem. 275, 27832-27837 (2000); and Yuan, Z. et al. Na/K-ATPase tethers phospholipase C and IP₃ receptor into a calcium-regulatory complex. Mol. Biol. Cell 16, 4034-4045 (2005), content of all of which is herein incorporated by reference. Several members of this complex have now been identified, including SRC kinase, epidermal growth-factor receptor (EGFR), inositol 1,4,5-triphosphate (IP3) receptor and caveolins. These are all engaged in the formation of this signalling domain, which is localized in the coated pits of the plasma membrane. Conformational changes on binding of cardiac glycosides trigger a downstream protein interplay that ultimately results in the activation of intracellular signal transduction cascades.

Interestingly, the signal transduction activity of this enzyme occurs through properties that are independent of its function as an ion pump. Segall, et al., Structural basis for α1 versus α2 isoform-distinct behavior of the Na, K-ATPase. J. Biol. Chem. 278, 9027-9034 (2003), content of which is herein incorporated by reference. Indeed, doses of cardiac glycosides—at concentrations that result in only subtle changes to the pumping activity of Na⁺/K⁺-ATPase—activate downstream signal transduction cascades and regulate many cellular processes including cell growth, cell motility, and apoptosis. See for example, Liu, et al., Role of caveolae in ouabain-induced proliferation of cultured vascular smooth muscle cells of the synthetic phenotype. Am. J. Physiol. Heart Circ. Physiol. 287, H2173-H2182 (2004); Barwe, et al. Novel role for Na⁺, K-ATPase in phosphatidylinositol 3-kinase signaling and suppression of cell motility. Mol. Biol. Cell 16, 1082-1094 (2005); and Wang, et al. Apoptotic insults impair Na⁺, K⁺-ATPase activity as a mechanism of neuronal death mediated by concurrent ATP deficiency and oxidant stress. J. Cell Sci. 116, 2099-2110 (2003), content of all of which is herein incorporated by reference.

The catalytic subunit of the Na⁺/K⁺-ATPase is expressed in various isoforms (α1, α2, α3) that are detectable by specific antibodies. The functional enzyme is comprised of an alpha and beta subunits; families of isoforms for both subunits exist. Na⁺,K⁺-ATPase is one of the members of the family of cation pumps. The other prominent members of this family include gastric H⁺/K⁺-ATPase, sarcoplasmic and endoplasmic reticulum Ca²⁺-ATPase, plasma membrane Ca²⁺-ATPase, and plasma membrane H⁺-ATPase of fungi and higher plants, as well as heavy metal pumps.

The X-ray crystal structure of Na⁺/K⁺-ATPase (at 3.5 Å resolution) has been reported in Morth, J. P. et al. Crystal structure of the sodium-potassium pump. Nature 450, 1043-1049 (2007). It is an oligomer composed of at least two polypeptides: the α-subunit and the β-subunit. The α-subunit is the catalytic moiety of the enzyme. Homologous to single-subunit P-type ATPases, it bears the binding sites for Na⁺, K⁺, Mg²⁺, ATP and the highly conserved cardiac glycoside-binding site. The binding site is formed by the extracellular loops of the M1/M2, M3/M4 and M5/M6 moieties, as recently revealed by elegant functional studies. See for example, Qiu, L. Y. et al. Reconstruction of the complete ouabain-binding pocket of Na, K-ATPase in gastric H, K-ATPase by substitution of only seven amino acids. J. Biol. Chem. 280, 32349-32355 (2005); Qiu, L. Y. et al. Conversion of the low affinity ouabain-binding site of non-gastric H, K-ATPase into a high affinity binding site by substitution of only five amino acids. J. Biol. Chem. 281, 13533-13539 (2006); and Dostanic-Larson, I. et al. Physiological role of the α1- and α2-isoforms of the Na⁺—K⁺-ATPase and biological significance of their cardiac glycoside binding site. Am. J. Physiol. Regul. Integr. Comp. Physiol. 290, R524-R528 (2006). Several additional regulatory sites are also found on the α-subunit, including phosphorylation sites for numerous signal transducing kinases (such as phosphoinositide 3-kinase (PI3K), protein kinase C(PKC) and PKA), caveolins and ankyrins.

The regulatory β-subunit is a single-span glycoprotein with a chaperone-like activity that is unique to the K⁺-counter-transporting P-type ATPases (Morth, J. P. et al. Crystal structure of the sodium-potassium pump. Nature 450, 1043-1049 (2007)). It is mainly important for the recruitment of the α-subunit to the plasma membrane and for the occlusion of potassium ions (Morth, et al. 2007) Finally, the FXYD proteins are single-span, type I transmembrane proteins, which are often associated with the α β-complex and seem to act as modulators of the kinetic properties of the pump (Geering, K. Function of FXYD proteins, regulators of Na, K-ATPase. J. Bioenerg. Biomembr. 37, 387-392 (2005)).

In some embodiments of the aspects described herein, the modulator does not significantly modulates an amiloride-sensitive sodium channel. An amiloride-sensitive sodium channel is a membrane-bound ion-channel that is highly sodium-selective, and does not allow the entry or exit of any potassium ions. It is a constitutively active ion-channel. Amiloride-sensitive sodium channels are also referred to as epithelial sodium channel (“ENaC”) and sodium channel non-neuronal 1 (“SCNN1”) in the art. The channel is characterized by its sensitivity to amiloride and derivatives thereof, such as phenamil and benzamil, by its small unitary conductance (approximately 5 pS), by its high selectivity for lithium and sodium, and by its slow kinetics. The amiloride-sensitive sodium channels have high affinity to the diuretic blocker amiloride. See for example, H. Garity, “Molecular properties of epithelial, amiloride-blockable Na⁺ channels”, FASEB J. 8 (8): 522-528 (1994); T. Le and M. H. Saier Jr, “Phylogenetic characterization of the epithelial Na⁺ channel (ENaC) family”, Mol. Membr. Biol. 13 (3): 149-157 (1996); and Lazdunski, et al., “Molecular cloning and functional expression of a novel amiloride-sensitive Na+ channel”. J. Biol. Chem. 270 (46): 27411-27414 (1995), contents of all of which are herein incorporated by reference. The amiloride-sensitive sodium channel plays a major role in the Na⁺- and K⁺-ion homeostasis of blood, epithelia and extraepithelial fluids by active Na⁺-ion reabsorption. In vertebrates, amiloride-sensitive sodium channels control reabsorption of sodium in kidney, colon, lung and sweat glands; they also play a role in salt taste perception.

The amiloride-sensitive sodium channel is a heteromultimeric protein composed of three homologous subunits: α, β, γ. See for example, J. Loffing and L. Schild, “Functional domains of the epithelial sodium channel”, J. Am. Soc. Nephrol. 16 (11): 3175-81 (2005), content of which is herein incorporated by reference in its entirety. Each of the α, β, and γ subunits vary in length from 650 to 700 amino acids. At the protein level, each subunit shares 35% amino acid identity with the others. Each of the subunits consists of two transmembrane helices and an extracellular loop. The amino- and carboxy-termini of all polypeptides are located in the cytosol. Structurally, the proteins that belong to this family consist of about 510 to 920 amino acid residues. They are made of an intracellular N-terminus region followed by a transmembrane domain, a large extracellular loop, a second transmembrane segment and a C-terminal intracellular tail (Snyder, et al., “Membrane topology of the amiloride-sensitive epithelial sodium channel”, J. Biol. Chem. 269 (39): 24379-24383 (1994), content of which is herein incorporated by reference in its entirety).

The α, β, and γ subunits share significant identity with degenerins, a family of proteins found in the mechanosensory neurons and interneurons of the nematode Caenorhabditis elegans. They are also homologous to FaNaCh, a protein from Helix aspersa nervous tissues, which corresponds to a neuronal ionotropic receptor for the Phe-Met-Arg-Phe-amide peptide.

Generally, amiloride-sensitive sodium channel proteins are expressed in low copy number, and, thus, typically, only a few hundred molecules are expressed per cell. Additionally, amiloride-sensitive sodium channel tissue distribution is restricted to a few organs including the apical membranes of aldosterone-responsive tissues (i.e., the distal part of the nephron of the kidney, the distal part of the colon, and the ducts of exocrine glands); the epidermis of the skin; in hair follicles; the lungs; and the nephron.

### Treatment Methods

Currently, clinical trials are being conducted to test the effects of anti-interferon antibodies for treating systemic lupus erythematosus. See for example, Ronnblom, L. & Elkon, K. B. Cytokines as therapeutic targets in SLE. Nat Rev Rheumatol 6, 339-647; Yao, Y. et al. Neutralization of interferon-alpha/beta-inducible genes and downstream effect in a phase I trial of an anti-interferon-alpha monoclonal antibody in systemic lupus erythematosus. Arthritis Rheum 60, 1785-96 (2009); and Zagury, D. et al. IFNalpha kinoid vaccine-induced neutralizing antibodies prevent clinical manifestations in a lupus flare murine model. Proc Natl Acad Sci USA 106, 5294-9 (2009). Inventor's discovery of modualtion of induction of IFNβ gene expression by modulating intracellular ion concentration provides a novel approach for treatment of SLE and various interfreion and/or TNF induced inflammation diseases, such as rheumatic diseases.

Without wishing to be bound by a theory, the inhibition of IFN-β gene induction and the TNF-β response by ion-channel modulators provide a novel treatment for human diseases where overproduction of interferon or aberrant TNF signaling is involved. For example, high levels of interferon production plays a major role in the autoimmune disease systemic lupus erythematosus (SLE). In SLE patients, the tolerance of autoantigen breaks down and high levels of IFN are detected in serum. This leads to aberrant activation of immature myeloid dendritic cells and downstream effector cells involved in autoimmune reactions. See, for example, Banchereau, J. & Pascual, V. Type I interferon in systemic lupus erythematosus and other autoimmune diseases. Immunity 25, 383-92 (2006), content of which is herein incorporated by reference. Chromosomal DNA and nucleosome are among the most common autoantigens detected in SLE patients. As discussed above, the inventors have discovered, inter alia, that ion-channel modulators are potent inhibitors of IFNβ gene activation by virus, dsRNA, and dsDNA.

Accordingly, in another aspect the invention provides a method for treating a subject suffering from a disease or disorder characterized by elevated levels of interferon-beta and/or elevated levels of interferon-beta gene expression, the method comprising administering an effective amount of a Na⁺, Ca²⁺, or K⁺ ion-channel modulator to the subject.

In some embodiments of the aspects described herein, the disease, disorder, or disease condition characterized by elevated levels of interferon-beta and/or elevated levels of interferon-beta gene expression is an autoimmune disease, neurodegenerative disease, inflammation, an inflammation associated disorder, a disease characterized by inflammation, or a pathogen or non-pathogen infection.

As used herein, the term “autoimmune disease” refers to disease or disorders wherein the immune system of a subject, e.g., a mammal, mounts a humoral or cellular immune response to the msubject's own tissue or to antigenic agents that are not intrinsically harmful to the subject, thereby producing tissue injury in such a subject. Examples of such disorders include, but are not limited to, systemic lupus erythematosus (SLE), mixed connective tissue disease, scleroderma, Sjögren's syndron, rheumatoid arthritis, and Type I diabetes.

As used herein, the term “neurodegenerative disease or disorder” includes any disease disorder or condition that affects neuronal homeostasis, e.g., results in the degeneration or loss of neuronal cells. Neurodegenerative diseases include conditions in which the development of the neurons, i.e., motor or brain neurons, is abnormal, as well as conditions in which result in loss of normal neuron function. Examples of such neurodegenerative disorders include Alzheimer's disease and other tauopathies such as frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-park-insonism-amytrophy complex, Pick's disease, or Pick's disease-like dementia, corticobasal degeneration, frontal temporal dementia, Parkinson's disease, Huntington's disease, amyotrophic lateral sclerosis (ALS), multiple sclerosis, Friedreich's ataxia, Lewybody disease, spinal muscular atrophy, and parkinsonism linked to chromosome 17.

As used herein, the term “inflammation” refers to any cellular processes that lead to the activation of caspase-1, or caspase-5, the production of cytokines IL-1 and IL-8, and/or the related downstream cellular events resulting from the actions of the cytokines thus produced, for example, fever, fluid accumulation, swelling, abscess formation, and cell death. As used herein, the term “inflammation” refers to both acute responses (i.e., responses in which the inflammatory processes are active) and chronic responses (i.e., responses marked by slow progression and formation of new connective tissue). Acute and chronic inflammation may be distinguished by the cell types involved. Acute inflammation often involves polymorphonuclear neutrophils; whereas chronic inflammation is normally characterized by a lymphohistiocytic and/or granulomatous response.

As used herein, the term “inflammation” includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation include, but are not limited to, diffuse inflammation, focal inflammation, croupous inflammation, interstitial inflammation, obliterative inflammation, parenchymatous inflammation, reactive inflammation, specific inflammation, toxic inflammation and traumatic inflammation.

As used herein, the term “pathogen infection” refers to infection with a pathogen. As used herein the term “pathogen” refers to an organism, including a microorganism, which causes disease in another organism (e.g., animals and plants) by directly infecting the other organism, or by producing agents that causes disease in another organism (e.g., bacteria that produce pathogenic toxins and the like). As used herein, pathogens include, but are not limited to bacteria, protozoa, fungi, nematodes, viroids and viruses, or any combination thereof, wherein each pathogen is capable, either by itself or in concert with another pathogen, of eliciting disease in vertebrates including but not limited to mammals, and including but not limited to humans. As used herein, the term “pathogen” also encompasses microorganisms which may not ordinarily be pathogenic in a non-immunocompromised host. Specific nonlimiting examples of viral pathogens include Herpes simplex virus (HSV) 1, HSV2, Epstein Barr virus (EBV), cytomegalovirus (CMV), human Herpes virus (HHV) 6, HHV7, HHV8, Varicella zoster virus (VZV), hepatitis C, hepatitis B, HIV, adenovirus, Eastern Equine Encephalitis Virus (EEEV), West Nile virus (WNE), JC virus (JCV) and BK virus (BKV).

As used herein, the term “microorganism” includes prokaryotic and eukaryotic microbial species from the Domains of Archaea, Bacteria and Eucarya, the latter including yeast and filamentous fungi, protozoa, algae, or higher Protista. The terms “microbial cells” and “microbes” are used interchangeably with the term microorganism.

As used herein, the term “Bacteria,” or “Eubacteria”, refers to a domain of prokaryotic organisms. Bacteria include at least 11 distinct groups as follows: (1) Gram-positive (gram+) bacteria, of which there are two major subdivisions: (i) high G+C group (Actinomycetes, Mycobacteria, Micrococcus, others) (ii) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram-positive bacteria” include cocci, nonsporulating rods, and sporulating rods. The genera of Grampositive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

As used herein, the term “specific defense system” is intended to refer to that component of the immune system that reacts to the presence of specific antigens. Inflammation is said to result from a response of the specific defense system if the inflammation is caused by, mediated by, or associated with a reaction of the specific defense system. Examples of inflammation resulting from a response of the specific defense system include the response to antigens such as rubella virus, autoimmune diseases such as lupus erythematosus, rheumatoid arthritis, Reynaud's syndrome, multiple sclerosis etc., delayed type hypersensitivity response mediated by T-cells, etc. Chronic inflammatory diseases and the rejection of transplanted tissue and organs are further examples of inflammatory reactions of the specific defense system.

As used herein, a reaction of the “non-specific defense system” is intended to refer to a reaction mediated by leukocytes incapable of immunological memory. Such cells include granulocytes and macrophages. As used herein, inflammation is said to result from a response of the nonspecific defense system, if the inflammation is caused by, mediated by, or associated with a reaction of the non-specific defense system. Examples of inflammation which result, at least in part, from a reaction of the non-specific defense system include inflammation associated with conditions such as: adult respiratory distress syndrome (ARDS) or multiple organ injury syndromes secondary to septicemia or trauma; reperfusion injury of myocardial or other tissues; acute glomerulonephritis; reactive arthritis; dermatoses with acute inflammatory components; acute purulent meningitis or other central nervous system inflammatory disorders; thermal injury; hemodialysis; leukophoresis; ulcerative colitis; Crohn's disease; necrotizing enterocolitis; granulocyte transfusion associated syndromes; and cytokine-induced toxicity. The term immune-mediated refers to a process that is either autoimmune or inflammatory in nature.

In some embodiments of the aspects described herein, the inflammation-associated disorder or disease characterized by inflammation is selected from the group consisting of asthma, autoimmune diseases, chronic prostatitis, glomerulonephritis, inflammatory bowl disease, pelvic inflammatory disease, reperfusion injury, arthritis, silicosis, vasculitis, inflammatory myopathies, hypersensitivities, migraine, psoriasis, gout, artherosclerosis, and any combinations thereof.

Exemplary inflammatory diseases include, but are not limited to, rheumatoid arthritis, inflammatory bowel disease, pelvic inflammatory disease, ulcerative colitis, psoriasis, systemic lupus erythematosus, multiple sclerosis, type 1 diabetes mellitus, multiple sclerosis, psoriasis, vaculitis, and allergic inflammation such as allergic asthma, atopic dermiatitis, and contact hypersensitivity. Other examples of auto-immune-related diseases or disorders, include but should not be construed to be limited to, rheumatoid arthritis, multiple sclerosis (MS), systemic lupus erythematosus, Graves' disease (overactive thyroid), Hashimoto's thyroiditis (underactive thyroid), Type 1 diabetes mellitus, celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, Wegener's granulomatosis, fibromyalgia (FM), autoinflammatory PAPA syndrome, Familial Mediaterranean Fever, familial cold autoinflammatory syndrome, Muckle-Wells syndrome, and the neonatal onset multisystem inflammatory disease.

As used herein, an anti-inflammation treatment aims to prevent or slow down (lessen) an undesired physiological change or disorder, such as the development or progression of the inflammation. Beneficial or desired clinical results include, but are not limited to, alleviation of symptoms, diminishment of extent of disease, stabilized (i.e., not worsening) state of disease, delay or slowing of inflammation disease progression, amelioration or palliation of the disease state, and remission (whether partial or total), whether detectable or undetectable. An anti-inflammation treatment can also mean prolonging survival as compared to expected survival if not receiving treatment. An anti-inflammation treatment can also completely suppress the inflammation response.

### Pharmaceutical Compositions

For administration to a subject, the ion-channel modulators can be provided in pharmaceutically acceptable compositions. These pharmaceutically acceptable compositions comprise a therapeutically-effective amount of one or more of the ion-channel modulators, formulated together with one or more pharmaceutically acceptable carriers (additives) and/or diluents. As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, contents of all of which are herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein means that amount of a compound, material, or composition comprising an ion-channel modulator which is effective for producing some desired therapeutic effect in at least a sub-population of cells in an animal at a reasonable benefit/risk ratio applicable to any medical treatment. For example, an amount of an ion-channel modulator administered to a subject that is sufficient to produce a statistically significant, measurable change in level of interferon-beta.

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and infrasternal injection and infusion. In preferred embodiments of the aspects described herein, the compositions are administered by intravenous infusion or injection.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, at least one symptom of a disease or disorder is alleviated by at least 5%, at least 10%, at least 20%, at least 30%, at least 40%, or at least 50%.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with autoimmune disease or inflammation.

In addition, the methods described herein can be used to treat domesticated animals and/or pets. A subject can be male or female. A subject can be one who has been previously diagnosed with or identified as suffering from or having a disorder characterized with elevated levels of interferon-beta and/or elevated levels of interferon-beta gene expression, or one or more complications related to such disease but need not have already undergone treatment.

A subject can be one who has been previously diagnosed with or identified as suffering from or having a disease or disorder characterized by elevated levels of interferon-beta and/or elevated interferon-beta gene expression.

For example, a subject can be diagnosed with systemic erythematosus lupus by having elevated levels of at least one autoantibody relative to the level of the autoantibody in a subject not diagnosed with systemic erythematosus lupus. Exemplary autoantibodies for diagnosis of systemic erythematosus lupus include, but are not limited to, antinuclear antibody (ANA), anti-double strand DNA antibody (anti-dsDNA), anti Sm nuclear antigen antibody (anti-Sm), anti-phospholipid antibody, and any combinations thereof. Such elevated levels can be at least 5%, at least 10%, at least 15%, at least 20%, at least 25%, at least 30%, at least 40%, at least 50%, at least 60%, at least 70%, at least 80%, at least 90%, at least 1-fold, at least 2-fold, at least 3-fold, at least 4-fold, at least 5-fold, or at least 10-fold or higher compared to a subject not diagnosed with systemic erythematosus lupus.

Alternatively, a subject can be diagnosed with systemic erythematosus lupus by having elevated levels of interferon-beta and or interferon-beta gene expression relative to levels in a subject not diagnosed with systemic erythematosus lupus. Such elevated levels can be at least 5%, at least 10%, at least 15%, at least 20%, at least 25%, at least 30%, at least 40%, at least 50%, at least 60%, at least 70%, at least 80%, at least 90%, at least 1-fold, at least 2-fold, at least 3-fold, at least 4-fold, at least 5-fold, or at least 10-fold or higher compared to a subject not diagnosed with systemic erythematosus lupus.

A subject can be one who is not currently being treated with an ion-channel modulator.

A subject can be one who has been previously diagnosed with a disease that is being treated with a therapeutic regime comprising an ion-channel modulator, wherein the disease is not a disease characterized by elevated levels of interferon-beta and/or elevated levels of interferon-beta gene expression. Accordingly, in some embodiments, the treatment method comprising adjusting the therapeutic regime of the subject such that at least one symptom of a disease characterized by elevated levels of interferon-beta and/or elevated levels of interferon-beta gene expression is reduced. Without limitation, a therapeutic regime can be adjusted by modulating the frequency of administration of the ion-channel modulator and/or by altering the site or mode of administration.

In some embodiments of the aspects described herein, the method further comprising diagnosing a subject for elevated levels of interferon-beta and/or elevated levels of interfern-beta gene expression prior to contacting a cell with the ion-channel modulator.

In some embodiments of the aspects described herein, the method further comprising selecting a subject with elevated levels of interferon-beta and/or elevated levels of interferon-beta gene expression prior to contacting a cell with the ion-channel modulator.

### Combination Therapy

The ion-channel modulator can be administrated to a subject in combination with a pharmaceutically active agent. Exemplary pharmaceutically active compound include, but are not limited to, those found in Harrison's Principles of Internal Medicine, 13^{th} Edition, Eds. T. R. Harrison et al. McGraw-Hill N.Y., N.Y.; Physicians Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety. In some embodiments of the aspects described herein, pharmaceutically active agent include those agents known in the art for treatment of autoimmune diseases, inflammation or inflammation associated disorders, or infections.

In some embodiments, the pharmaceutically active agent is an anti-interferon agent. Without limitation, anti-interferon agents include anti-interferon antibodies or fragments or derivatives thereof. Exemplary anti-interferon antibodies include, but are not limited to, those described in Ronnblom, L. & Elkon, K. B. Cytokines as therapeutic targets in SLE. Nat Rev Rheumatol 6, 339-647; Yao, Y. et al. Neutralization of interferon-alpha/beta-inducible genes and downstream effect in a phase I trial of an anti-interferon-alpha monoclonal antibody in systemic lupus erythematosus. Arthritis Rheum 60, 1785-96 (2009); and Zagury, D. et al. 1FNalpha kinoid vaccine-induced neutralizing antibodies prevent clinical manifestations in a lupus flare murine model. Proc Natl Acad Sci USA 106, 5294-9 (2009), those described in U.S. Pat. No. 4,902,618; No. 5,055,289; No. 7,087,726; and No. 7,741,449, and those described in U.S. patent application Ser. No. 10/440,202; No. 11/342/020; and No. 12/517,334, content of all of which is herein incorporated by reference.

The ion-channel modulator and the pharmaceutically active agent can be administrated to the subject in the same pharmaceutical composition or in different pharmaceutical compositions (at the same time or at different times). When administrated at different times, the ion-channel modulator and the pharmaceutically active agent can be administered within 5 minutes, 10 minutes, 20 minutes, 60 minutes, 2 hours, 3 hours, 4, hours, 8 hours, 12 hours, 24 hours of administration of the other When the ion-channel modulator and the pharmaceutically active agent are administered in different pharmaceutical compositions, routes of administration can be different.

### Dosage

The amount of ion-channel modulator that can be combined with a carrier material to produce a single dosage form will generally be that amount of the ion-channel modulator that produces a therapeutic effect. Generally out of one hundred percent, this amount will range from about 0.01% to 99% of ion-channel modulator, preferably from about 5% to about 70%, most preferably from 10% to about 30%.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment. Generally, the compositions are administered so that ion-channel modulator is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 tmg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

In some embodiments, the compostions are administered at a dosage so that ion-channel modulator or a metabolite thereof has an in vivo concentration of less than 500 nM, less than 400 nM, less than 300 nM, less than 250 nM, less than 200 nM, less than 150 nM, less than 100 nM, less than 50 nM, less than 25 nM, less than 20, nM, less than 10 nM, less than 5 nM, less than 1 nM, less than 0.5 nM, less than 0.1 nM, less than 0.05, less than 0.01, nM, less than 0.005 nM, less than 0.001 nM after 15 mins, 30 mins, 1 hr, 1.5 hrs, 2 hrs, 2.5 hrs, 3 hrs, 4 hrs, 5 hrs, 6 hrs, 7 hrs, 8 hrs, 9 hrs, 10 hrs, 11 hrs, 12 hrs or more of after administration.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments of the aspects described herein, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

### DEFINITIONS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “elevated,” as used in conjunction with elevated interferon-beta levels or elevated interferon-beta gene expression, means an increase by a statically significant amount; for the avoidance of any doubt, the term “elevated” means an increase of at least 5%, at least 10%, at least 15%, at least 20%, at least 25%, at least 30%, at least 40%, at least 50%, at least 60%, at least 70%, at least 80%, at least 90%, at least 95%, at least 1-fold, at least 1.5-fold, at least 2-fold, at least 3-fold, at least 4-fold, at least 5-fold, or at least 10-fold or greater as compared to a reference level.

The term “statistically significant” or “significantly” refers to statistical significance and generally means a two standard deviation (2SD) above or below a reference level. The term refers to statistical evidence that there is a difference. It is defined as the probability of making a decision to reject the null hypothesis when the null hypothesis is actually true. The decision is often made using the p-value.

As used herein, the term “ex vivo” refers to cells which are removed from a living organism and cultured outside the organism (e.g., in a test tube).

As used herein, the term “pharmaceutically-acceptable salts” refers to the conventional nontoxic salts or quaternary ammonium salts of the ion-channel modulators, e.g., from non-toxic organic or inorganic acids. These salts can be prepared in situ in the administration vehicle or the dosage form manufacturing process, or by separately reacting a purified ion-channel modulator in its free base or acid form with a suitable organic or inorganic acid or base, and isolating the salt thus formed during subsequent purification. Conventional nontoxic salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), content of which is herein incorporated by reference in its entirety.

In some embodiments of the aspects described herein, representative salts include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like.

As used herein, a “prodrug” refers to compounds that can be converted via some chemical or physiological process (e.g., enzymatic processes and metabolic hydrolysis) to an ion-channel modulator. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11, 345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenyloin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), content of all of which is herein incorporated by reference in its entirety.

All patents and other publications identified are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Materials and Methods

Cells, Reagents and Plasmids:

293T, Hela, MG63, and Namalwa cells were obtained from ATCC, and wild type MEFs were obtained from Chen Yeh (University of Toronto, Toronto, Canada). Bufalin was purchased from Calbiochem, Digoxin, ouabain, Diazoxide, Nimodipine, phenamil, poly dA:dT and poly I:C were purchased from Sigma. The ion-channel ligand library, Biomol Green reagents were obtained from Biomol. The high content small molecule library has been described before in Chen, S. et al. A small molecule that directs differentiation of human ESCs into the pancreatic lineage. Nat Chem Biol 5, 258-65 (2009), content of which is herein incorporated by reference. Recombinant TNFalpha was from Roche. pEF-BOS Flag-RIG-I was a gift from Dr. T. Fujita (Kyoto University, Japan), and mouse ATP1a1, ATP1a3, human ATP1a1 expression constructs were purchased from OpenBiosystems. Constructs for mutant RIG-I (K270A) and mouse ATP1a1 (D376E) were generatd by standard site-directed mutagenesis. TBK1 expression construct has been described before in Fitzgerald, K. A. et al. IKKepsilon and TBK1 are essential components of the IRF3 signaling pathway. Nat Immunol 4, 491-6 (2003), content of which is herein incorporated by reference. The MAVS expression construct was generated by cloning mouse MAVS cDNA into pKH3 vector.

Antibodies and Western Blots:

Antibodies against human IRF3, ATP1a1, STAT1, Traf6, HSP70 and p656 were from Santa Cruz, RIG-I, MDA5 PARP1, cleaved PARP1 (human specific), cleaved Caspase3, phosp-IKBα Ser 32/36, phosphor-S276, S468 and S536 p65 antibodies were Cell Signaling, β-actin antibody was from Abcam. The IKBα antibody was from IMGenex. Trex1 antibody was from BD Biosciences. Western blots were carried out according to standard protocols. About 50 ug of total protein lysate (lysed in a buffer of 20 mM Tris-HCl, pH7.5, 150 mM NaCl, 1% Triton X-100, 1 mM EDTA, 30 mM NaF, 1 mM glycerolphosphate, 1× proteinase inhibitor (Roche) and 1 mM Na₃VO₄) was denatured in sampling buffer (50 mM Tris-HCl, pH 6.8, 10% glycerol, 2% SDS, 0.02% bromophenal blue and 2.5% β-mercaptoethanol) and subjected to SDS-PAGE. Proteins were transferred to a PVDF membrane, blocked with 5% milk in Tris-buffered saline tween 20 (TBST), and incubated with various primary antibodies solutions. Washed membranes were incubated with HRP conjugated secondary antibody and protein bands visualized with ECL reagents (Millipore or Pierce).

Luciferase Reporter Assay and Chemical Treatment:

Approximately 40,000 293T cells were seeded in a 96 well plate, and co-transfected with IFNβ-110 firefly luciferase reporter and renilla luciferase plasmids (Fitzgerald, K. A. et al. IKKepsilon and TBK1 are essential components of the IRF3 signaling pathway. Nat Immunol 4, 491-6 (2003)). After 24 hrs, cells were treated with various chemicals at the indicated concentrations, and sendai virus infection was initiated one hour later. After another 24 hrs, cells were lysed and subjected to Dual-Glo luciferase assay analysis (Promega) with an Analyst AD plate reader.

In Vitro RNA Binding and ATPase Assay:

Double strand RNA corresponding to GFP sequences (67 bp of the 3′ end) was generated by in vitro transcription with T7 RNA polymerase. About 200 ng of dsRNA was incubated with 0.5 ug of recombinant Flag-tagged RIG-I protein in a 20 ul buffer of 20 mM Tris-HCl, pH8.0, 1.5 mM MgCl₂, 1.5 mM DTT and 5% glycerol for 15 min at room temperature. RNA:protein complexes were separated in a 0.8% agarose gel with 0.5×TBE running buffer, and run for 1.5 hrs at 100 volts. For the ATPase assay, the same RNA:protein complexes were formed by incubation at 37° C. for 15 min, then ATP added to a final concentration of 1 mM and further incubated for 15 min. The released free phosphates were measured with the BIOMOL GREEN kit (Biomol) according to the manufactures instructions.

Biotin-Labeled dsRNA Pull Down Assay:

For the biotin-labeled dsRNA pull down assay, the inventors generated dsRNA (GFP sequences) by in vitro T7 RNA polymerase transcription in the presence of biotin-11-UTP (Ambion). About 8 ug of this dsRNA was transfected into 2 million 293T/RIG-I stable cells treated with/without bufalin. 6 hrs later, cell lysates were prepared and subjected to NeutrAvidin beads (Pierce) pull down for 1 hr at 4° C. Bound protein was separated by SDS-PAGE and transferred to PVDF membrane. Binding of these RNAs by RIG-I was monitored by probing the membrane with an anti-RIG-I antibody.

Virus Infection, RNA Preparation, Immunofluorescent Staining:

Sendai virus infection was carried out as described in McWhirter, S. M. et al. IFN-regulatory factor 3-dependent gene expression is defective in Tbk1-deficient mouse embryonic fibroblasts. Proc Natl Acad Sci USA 101, 233-8 (2004), concentrated virus stock (Cantell strain, Charles River Lab) was added to cultured cells at a concentration of 200-300 HAU/ml and incubated for the indicated times before harvesting the cells for protein or RNA analysis. Total RNA was extracted with Trizol reagent (invitrogen). RT-PCR and real time quantitative PCR were conducted according to standard protocols. For microarray experiments, RNA was biotin-labeled with the Illumina TotalPrep RNA Amplification Kit (Ambion) and subjected to the Illumina HumanRef-8 v3.0 BeadChip microarray analysis. Immunofluorescent staining was conducted according to standard protocols: cells were fixed with 4% formaldehyde for 10 min, washed with PBS and permeabilized with 0.1% Triton X-100 in PBS, and incubated with primary antibodies over night. Cells were extensively washed before incubating with FITC-conjugated secondary antibody, mounted with DAPI containing media, and subjected to Epifluorescent or confocal microscopy.

Lentivirus Mediated shRNA Knockdown:

shRNA constructs were generated by cloning sequences 5′-ccggaaagactgaaagaatac-3′ targeting human ATP1a1 mRNA, or 5′-gtgattcgaaatggagagaaa-3′ targeting mouse ATP1a1 mRNA into the pLK0.1 TRC cloning vector, a construct with scramble sequences was used as control. Packaging of lentivirus was achieved by co-transfecting 293T cells with targeting plasmid together with pLP1, pLP2 and pLP-VSVG plasmids according to the Viralpower Lentivirus expression system from Invitrogen. The supernatants from transfected cells was harvested and used to infect new cells. Knockdown cells were pooled by puromycin selection and the efficiency of knocking down was verified by western blot.

Intracellular Sodium Concentration Measurements:

The intracellular sodium concentration was measured using the fluorescent dye SBFI (Minta, A. & Tsien, R. Y., Fluorescent indicators for cytosolic sodium, J. Biol. Chem. 264: 19449-57 (1989)), with some minor modifications from the published procedures (Ishikawa, S., Fujisawa, G., Okada, K., & Saito, T., Thapsigargin increases cellular free calcium and intracellular sodium concentrations in cultured rat glomerular mesangial cells, Biochem. Biophys. Res. Comm. 194: 287-293 (1993)). 293T cells were harvested and washed with physiological saline solution (PSS, 140 mM NaCl, 5 mM KCl, 1 mM MgCl₂, 2 mM CaCl₂, 10 mM glucose and 10 mM HEPES, pH7.5). Cells were then resuspended in the same buffer containing 10 μM SBFI-AM (Invitrogen) and 0.02% Pluronic F-127 (Invitrogen) and incubated for 1 hour at 37° C. Free SBFI-AM were washed away with PSS buffer. Cells were then resuspended in PSS buffer with and without bufalin (1 μM) and incubated for 30 minutes at 37° C. Fluorescence emission at 525 nm from excitation of 340 nm and 380 nm was recorded with a Spectramax Plus 384 plate reader and the ratio calculated. For the sodium concentration standard curve, SBFI-AM loaded cells were incubated with reference solutions containing different concentrations of sodium in the presence of 10 μM gramicidin at 37° C. for 30 minutes and subjected to the same fluorescence measurements. Reference solutions were based on the compositions of the PSS solutions, with different combinations of NaCl and KCl to make the total sodium and potassium concentration 150 mM.

Cell Viability Assay:

About 40,000 293T cells were seeded in each well of a 96-well plate in 100 μl culture medium and treated with increasing amounts of bufalin. 8 hours later, 20 μl of the CellTiter-Blue reagent (Promega) was added to each well and incubated for two more hours at 37° C. Fluorescence was recorded from 560 nm excitation/590 nm emission with a Spectramax Plus 384 plate reader. For CellTiter-Glo Luminescent viability assay, 100 CellTiter-Glo reagent (Promega) was added to each well and mixed, and incubated for 10 minutes at room temperature. Luminescence was measured with an Analyst AD plate reader.

Flow Cytometry Analysis of Apoptosis:

293T cells were treated with 1 μM bufalin or DMS for 8 hours, or 4 μM sturosporine for 4 hours. Treated cells and untreated control cells were harvested and washed with PBS, and stained with APC conjugated Annexin V and 7-AAD (both from BD Biosciences) in Annexin V binding buffer (10 mM HEPES, pH 7.4, 140 mM NaCl, 2.5 mM CaCl₂) for 15 minutes at room temperature, and then subjected to flow cytometry analysis on a FACSCalibur (BD Biosciences).

Native Gel Analysis:

Native PAGE analysis was conducted according to published procedures (Mori, et al., Identification of Ser-386 of interferon regulatory factor 3 as critical target for inducible phosphorylation that determines activation, J. Biol. Chem. 279: 9698-9702 (2004)). Briefly, total protein lysates were prepared with a lysis buffer containing 20 mM Tris-Hcl, pH 7.5, 150 mM NaCl, 1% Triton X-100, 1 mM EDTA, 30 mM NaF, 1 mM glycerolphosphate, 1× proteinase inhibitor (Roche) and 1 mM Na₃VO₄. About 5 μl of the lysate was mixed with equal volume of 2× loading buffer (125 mM Tris-Cl pH 6.8, 30% glycerol, 0.002% bromophenol blue), loaded onto a 7.5% native gel and electrophoresed at 25 mA for 50 minutes at 4° C. The gel was pre-run at 45 mA for 30 minutes at 4° C., with 0.2% deoxycholate in the cathode buffer. Proteins from the gel were transferred to a PVDF membrance and probed with anti-IRF3 or anti-RIG-I antibodies.

### Sequences of Primers:

The sequences of primers used in this study are listed in Table 1.

TABLE 1
Sequences of primers used in this study (in the
order of 5′ to 3′).

| | |
|---|---|
| Human IFNb: | GCTGCAGCTGCTTAATCTCC |
| | and |
| | TCCTCCAAATTGCTCTCCTG |
| Human Cxcl 10: | AAGGATGGACCACACAGAGG |
| | and |
| | TGGAAGATGGGAAAGGTGAG |
| Human GAPDH: | CTGACTTCAACAGCGACACC |
| | and |
| | GGTGGTCCAGGGGTCTTACT |
| Human IFNa8: | ACCCAGGTTAAGGGTCATCC |
| | and |
| | ATCAAGGCCCTCCTGTTACC |
| Human TNF: | CCTGTGAGGAGGACGAACAT |
| | and |
| | AGGCCCCAGTTTGAATTCTT |
| Human RIG-I: | CAAACCAGAGGCAGAGGAAG |
| | and |
| | CCAAGGCTTTGCACTTTCTG |
| Human ISG15: | TGTCGGTGTCAGAGCTGAAG |
| | and |
| | GCCCTTGTTATTCCTCACCA |
| Human CCL5: | CGCTGTCATCCTCATTGCTA |
| | and |
| | TGTACTCCCGAACCCATTTC |
| Human IFIT2: | ATTGCCAAAATGCGACTTTC |
| | and |
| | ATTTCAGCTCCCTTTCAGCA |
| Human IL8: | CTGCGCCAACACAGAAATTA |
| | and |
| | ATTGCATCTGGCAACCCTAC |
| Human OASL: | ACCTGAGGATGGAGCAGAGA |
| | and |
| | CAGCTTAGTTGGCCGATGTT |
| Human JUN: | CGAAAAAGGAAGCTGGAGAG |
| | and |
| | CCGACGGTCTCTCTTCAAAA |
| Human PRDM4: | GACTGGGAGGGAAGTGTCAA |
| | and |
| | GCTGTGTCCCAATCCATTCT |
| Human TMEM60: | GGATGAGAAAGCACCTTGGA |
| | and |
| | AG CAAGGCCCATAAAGGAAT |
| Human OVGP1: | GTGTGGACATTGGACATGGA |
| | and |
| | CCTGGGGGCAAAATCTTACT |
| Human LINS1: | CCTGGATTTGCTTGAGCTTC |
| | and |
| | GCATTAAGGCAGGCACAGAT |
| Human TXNIP1: | GCCACACTTACCTTGCCAAT |
| | and |
| | TTGGATCCAGGAACGCTAAC |
| Human PPP1R15A: | GATCAGCCGAGGATGAAAGA |
| | and |
| | TCCTCAGCAGCTTCCTCTTC |
| Human CITED2: | CGACGAGGAAGTTCTTATGTCC |
| | and |
| | AATTCACGCCGAAGAAGTTG |
| Human PRMT6: | GACCACATACATCATAGGGTGCT |
| | and |
| | GGGCTAGGCTCAGAAACCTC |
| Mouse IFNb: | CCCTATGGAGATGACGGAGA |
| | and |
| | CTGTCTGCTGGTGGAGTTCA |
| Mouse Cxcl10: | TCATCCTGCTGGGTCTGAGT |
| | and |
| | TTTTGGCTAAACGCTTTCATT |
| Mouse β-Actin: | CCTCTATGCCAACACAGTGC |
| | and |
| | ACATCTGCTGGAAGGTGGAC |
| Mouse IRF7: | TGCAAGGTGTACTGGGAGGT |
| | and |
| | TCACCAGGATCAGGGTCTTC |
| Mouse RIG-I: | AGAGCCAGCGGAGATAACAA |
| | and |
| | CCTTGATCATGTTCGCCTTT |
| Mouse Stat1: | GACCACCTCTCTTCCTGTCG |
| | and |
| | TGCCAACTCAACACCTCTGA |
| Mouse trex1: | GAGCAAAGCTGAGCTGGAAG |
| | and |
| | GCTGCTAGCTTGTTCCAAGG |
| Mouse CCL5: | CCCTCACCATCATCCTCACT |
| | and |
| | GGGAAGCGTATACAGGGTCA |
| Sendai virus NP: | GCTCACTCATTAGACACAGATAAGCAGCAC |
| | and |
| | GAAAAGCGGACTCTTGTTGACCATAGG |
| Sendai virus L: | TGATGTCAATGGGCAGAGAG |
| | and |
| | CATGCAGTACAACTTGATCATCC |

### Example 1

### Bufalin Inhibits Virus Induction of IFNβ Gene Expression

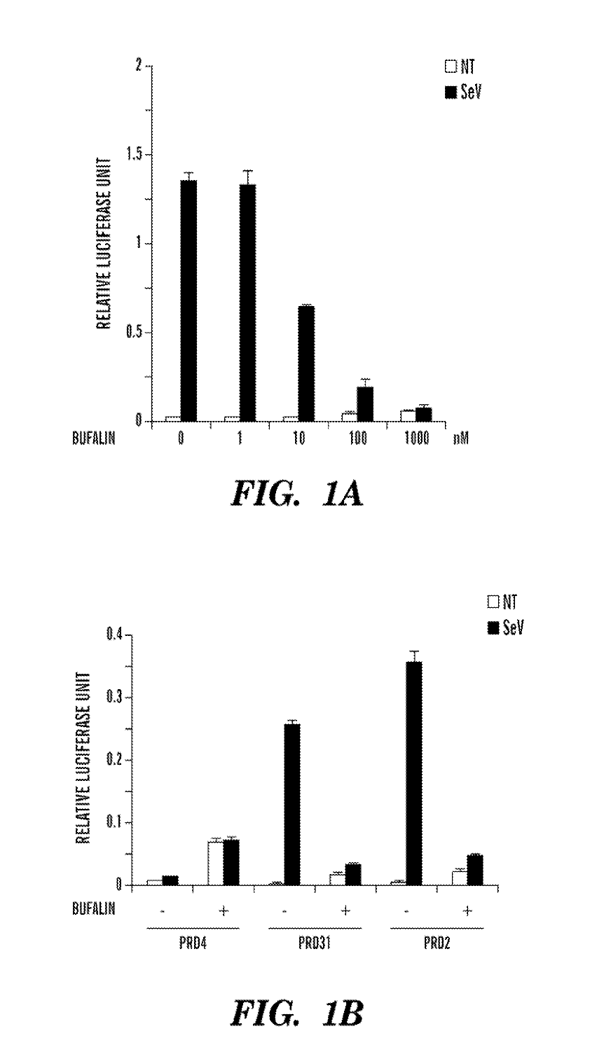

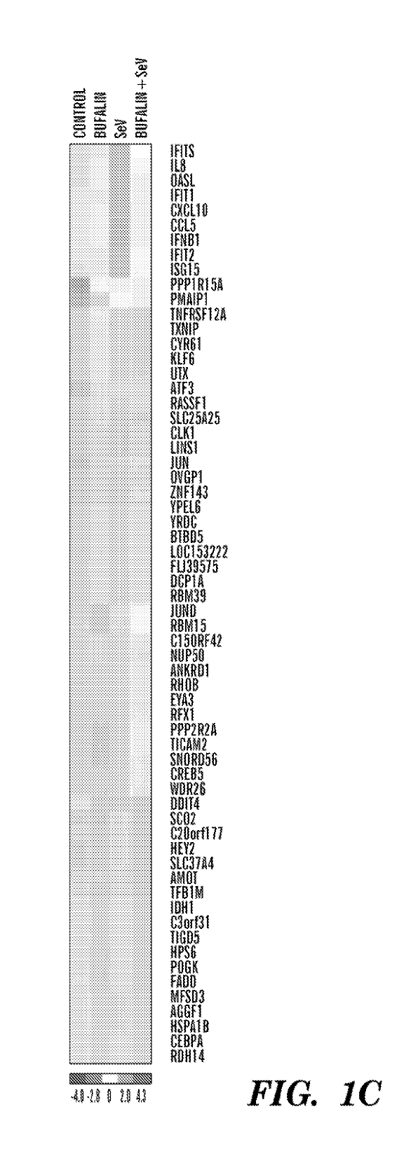

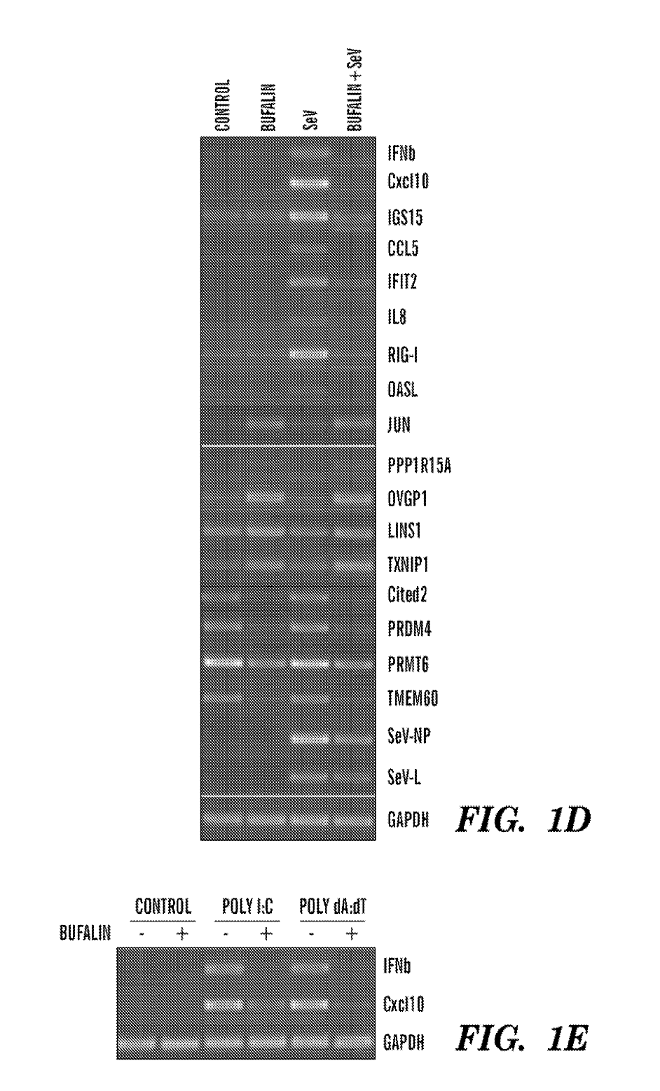

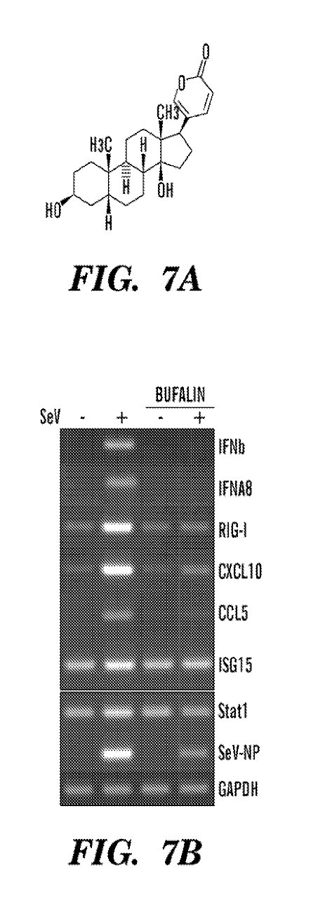

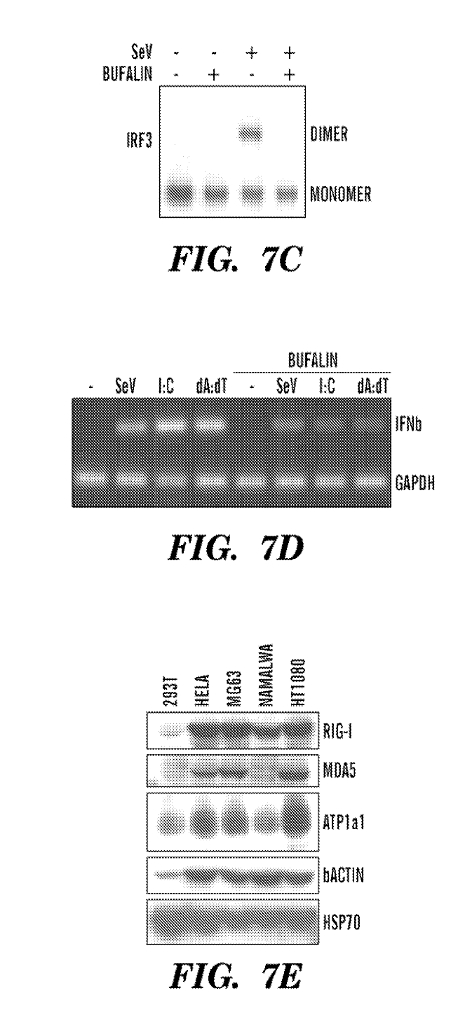

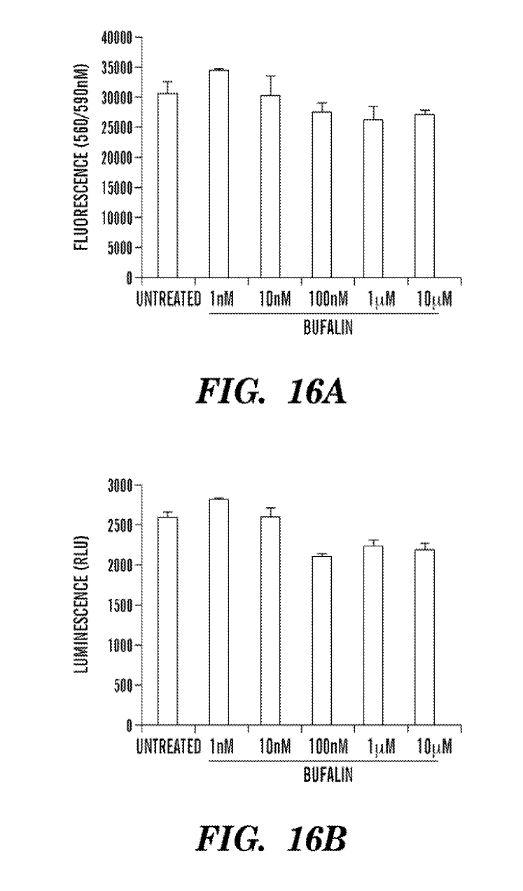

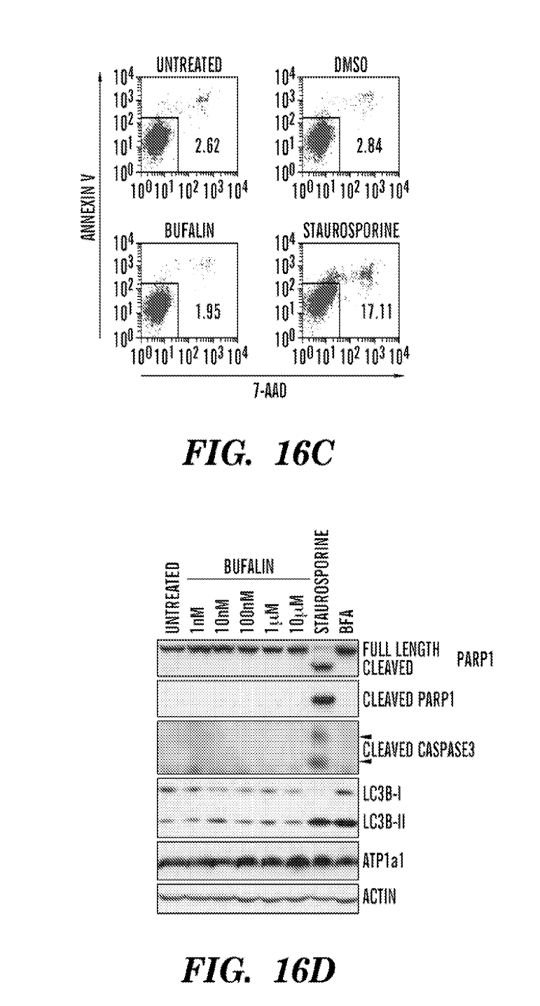

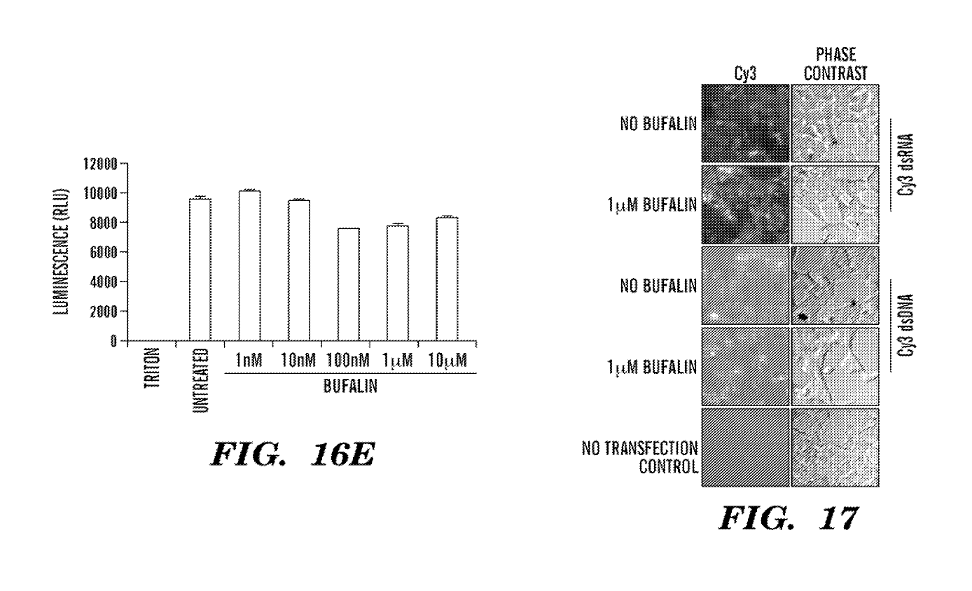

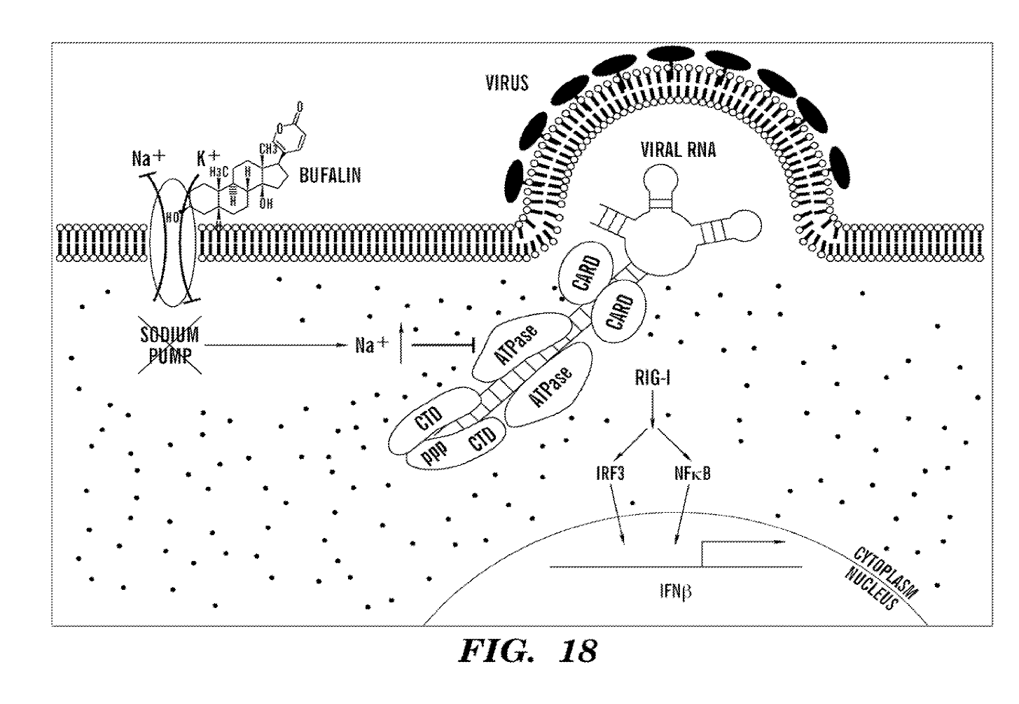

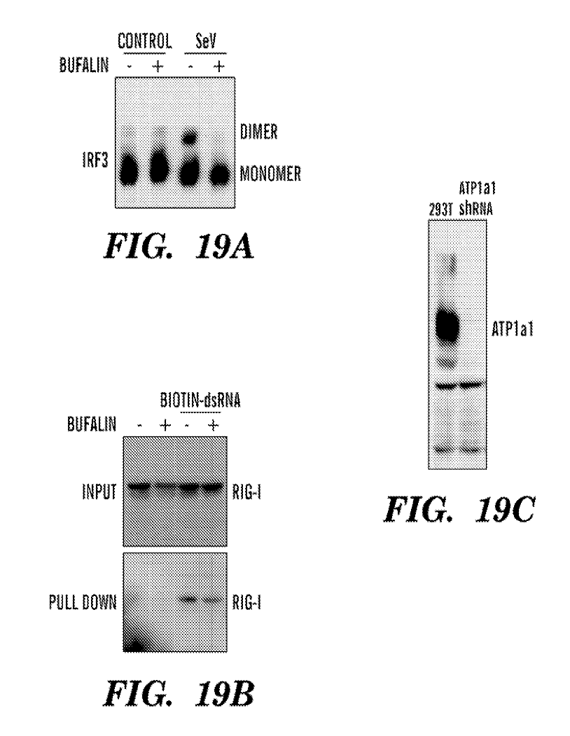

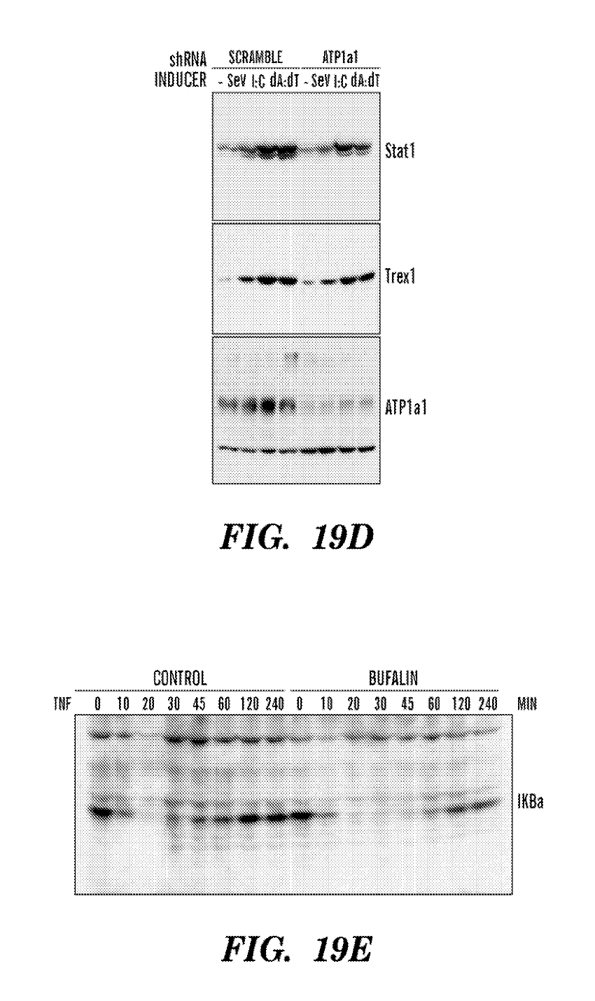

The inventors utilized a virus inducible luciferase reporter assay system to screen for small molecule inhibitors of IFNβ gene expression. Human 293T cells were transfected with the IFNβ promoter driving the expression of luciferase construct. The cells were treated with a library of chemical compounds, and sendai virus (SeV) infection initiated one hour later. Luciferase activity was measured after another 24 hrs of culture. Signals were normalized to the samples not treated with chemicals. Screening a high content chemical library with 478 compounds identified small molecules with either stimulatory or inhibitory effects on IFNβ gene expression. Some exemplary hit compounds that inhibit virus induced IFNβ expression are listed in Table 2. One compound, bufalin (FIG. 7A), a cardiac glycoside inhibitor of the sodium pump (Prassas, I. & Diamandis, E. P. Novel therapeutic applications of cardiac glycosides. Nat Rev Drug Disc 7, 926-35 (2008), content of which is herein incorporated by reference), consistently and strongly inhibited virus induction of IFNβ gene expression. While the initial screen was conducted at a concentration of 10 uM of each molecule, subsequent tests with different concentrations showed that Bufalin exerts inhibitory effects in a dose-dependent manner (FIG. 1A). The 50% inhibition (IC₅₀) of bufalin was calculated to be 4.3 nM and a concentration of 1 uM is sufficient to inhibit IFNβ expression by more than 90%. Without wishing to be bound by a theory, higher concentrations (1 and 10 uM) of Bufalin weakly induced IFN expression in the absence of virus infection, however the robust viral induction of IFN was completely suppressed at these concentrations. Without wishing to be bound by a theory, it can be pointed out that inhibition of IFNβ induction by bufalin is not due to the loss of cell viability (FIGS. 16A, 16B and 16E) or to the induction of cell death by apoptosis or autophagy (FIGS. 16C and 16D).

TABLE 2
Inhibition of virus induced IFNβ expression by some exemplary hit compounds
from the chemical library screen.

| Compound | Structure | Inhibition % @ 10 μM |
|---|---|---|
| Bufalin | 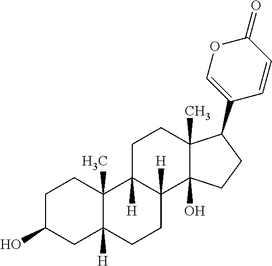 | 99.2 |
| Go 6976 | 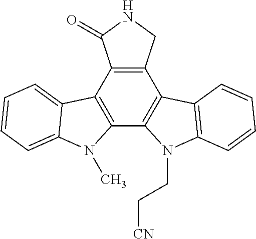 [SMILES: Cn1c2ccccc2c2c3c(c4c5ccccc5n(CCC#N)c4c21)CNC3=O] | 55.9* |
| Colcemid | 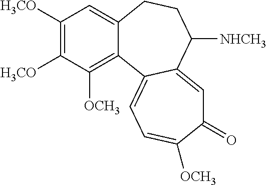 [SMILES: CNC1CCc2cc(OC)c(OC)c(OC)c2-c2ccc(OC)c(=O)cc21] | 52.7 |
| SB 225002 | 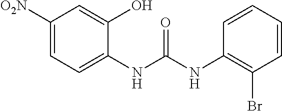 [SMILES: O=C(Nc1ccc([N+](=O)[O-])cc1O)Nc1ccccc1Br] | 50.8 |
| EGF receptor inhibitor | 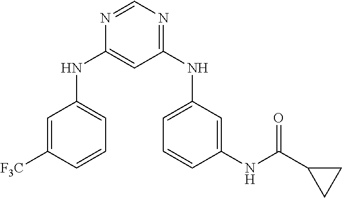 [SMILES: O=C(Nc1cccc(Nc2cc(Nc3cccc(C(F)(F)F)c3)ncn2)c1)C1CC1] | 38.5 |
| EGFR/ErbB-2/ErbB-4 Inhibitor | 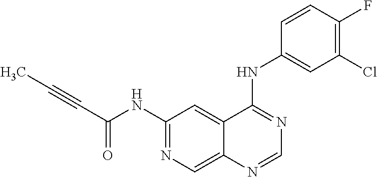 [SMILES: CC#CC(=O)Nc1cc2c(Nc3ccc(F)c(Cl)c3)ncnc2cn1] | 20.6 |

*tested at 3 μM

The IFNβ virus-inducible enhancer contains four positive regulatory domains (PRD), corresponding to binding sites for the transcription factors cJUN/ATF2 (PRDIV), IRF3/7(PRDIII/I) and NFκB (PRDII) respectively. All of these sites are required for the activation of IFNβ gene expression, and each transcription factor is activated through distinct signal transduction pathways. See for example, Maniatis, T. et al. Structure and function of the interferon-beta enhanceosome. Cold Spring Harb Symp Quant Biol 63, 609-20 (1998) and Thanos, D. & Maniatis, T. Virus induction of human IFN beta gene expression requires the assembly of an enhanceosome. Cell 83, 1091-100 (1995), content of both of which is herein incorporated by reference. In order to identify which signaling pathways is the target for bufalin, the inventors transfected 293T cells with luciferase reporters driven by multiple copies of PRDIV, PRDIII/I or PRDII elements, and tested the effects of bufalin on viral induction. SeV did not induce the PRDIV reporter in 293T cells, however bufalin treatment weakly induced the activity of PRDIV in the absence of virus infection (FIG. 1B), demonstrating that this element is not the target for inhibition. By contrast, viral induction of both the PRDIII/I and PRDII reporters was strongly inhibited by bufalin. With 1 uM of bufalin, viral induction of luciferase expression was >86% repressed compared with no drug treatment with both reporters (FIG. 1B). Without wishing to be bound by theory, bufalin can either inhibit IRF3/7 and NFκB activation separately and/or it can target signaling events at or before the bifurcation of the IRF and NFκB signaling pathways.

### Example 2

### Bufalin Blocks the Activation of Virus Inducible Genes

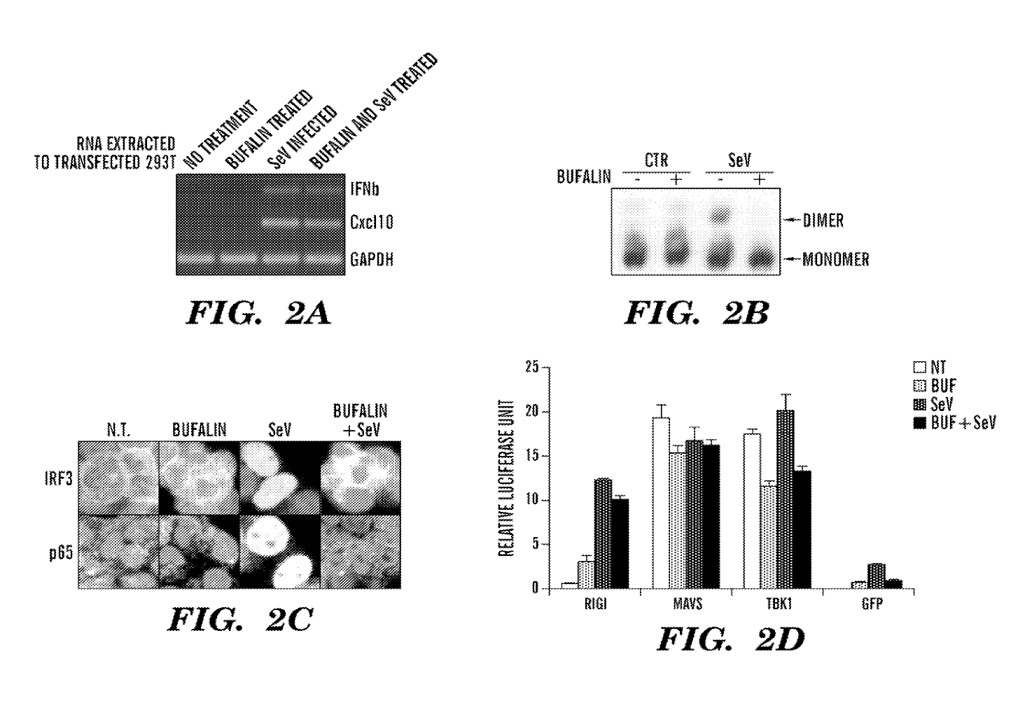

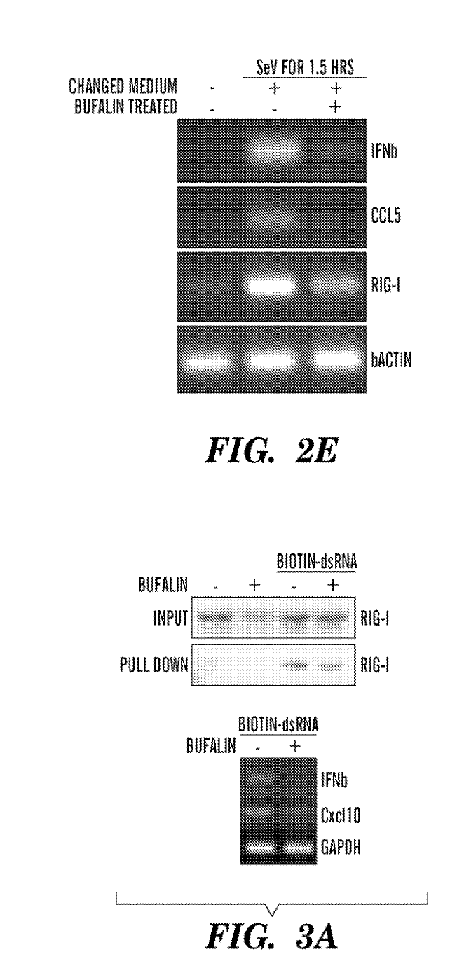

Virus infection leads to the activation of a large number of genes in addition to IFNβ. See for example, Hemmi, H. et al. The roles of two IkappaB kinase-related kinases in lipopolysaccharide and double stranded RNA signaling and viral infection. J Exp Med 199, 1641-50 (2004), content of which is herein incorporated by reference. The inventors investigated the effect of bufalin on this virus-inducible gene expression program. To accomplish this the inventors carried out a microarray analysis with 293T cells treated with bufalin and SeV either alone or in combination, and compared the genome wide expression profiles of all the samples. Interestingly, bufalin alone stimulated the expression of >80 genes to a level at least two-fold higher than the control (FIGS. 1C and 1D), including the genes encoding transcription factors that bind to PRDIV (cJUN and ATF2). Without wishing to be bound by theory, this can be one of the reasons the PRDIV reporter is weakly activated by bufalin (FIG. 1B). Treatment with bufalin alone lead to a decrease in the expression of over 40 genes by at least 2-fold (FIGS. 1C and 1D). By contrast, a typical cytokine and ISG expression profile was observed when cells were infected with virus (FIG. 1C). IFNβ, IL-8, IFIT1, IFIT2, IFIT3, ISG15, OASL, CXCL10, and CCL5 genes were among the highest virus-induced genes. Strikingly, bufalin completely blocked the induction of these genes. The expression profile from the bufalin and virus treated samples was similar to the samples treated with bufalin alone (FIG. 1C). Although slightly diminished, transcripts from the infecting virus were readily detected (SeV nucleocapsid, NP and RNA polymerase gene, L) in infected cells treated with bufalin (FIG. 1D). Moreover, when total RNAs extracted from virus infected cells or cells treated with bufalin and SeV were transfected into new 293T cells, both transfections strongly induced the expression of IFNβ and CXCL10 genes (FIG. 2A). These data demonstrate that bufalin does not block virus infection, nor eliminates the viral pathogen associated molecular pattern (PAMP) associated with virus infection, however, expression of the cellular innate antiviral genes was almost completely abolished by bufalin.

The inventors also discovered that bufalin similarly inhibits IFNβ gene expression when cells were infected with virus and then treated with bufalin (FIG. 2E). 293 cells were first infected with SeV (200 HAU/ml) and 1.5 hours later, virus containing medium was replaced by fresh medium with or without addition of bufalin (to a final concentration of 1 μM) and further incubated for 6 hours. Total RNA was extracted for the analysis of IFN-beta, RIG-1, and beta-actin expression by RT-PCR (FIG. 2E).

The inventor also tested the effects of bufalin on other inducers of IFNβ expression. For example, bufalin strongly inhibited IFNβ and CXCL10 gene expression in response to treatment of cells with double strand RNA (poly I:C) and double strand DNA (poly dA:dT) (FIG. 1E). This inhibition was not due to a reduction in transfection efficiency, as Cy3 labeled dsRNA and dsDNA were similarly transfected into bufalin treated and non-treated cells (FIG. 17). Cy3 labeled dsRNA (poly I:C) or dsDNA (poly dA:dT) were transfected into 293T cells pretreated with and without bufalin, medium was changed after 6 hours, and cell images under Cy3 channel or direct phase contrast were taken 8 hours after transfection.

To address the possibility that bufalin inhibition occurs only in 293T cells, the inventors tested other human cell lines. Strong inhibition of virus-induced IFNβ gene expression was observed with 1 uM of bufalin with the human B cell line Namalwa (FIG. 7B). The inventor also discovered that 1 uM bufalin inhibits virus induced IFNβ expression in Hela and MG63 cells, but less than observed with 293T and Namalwa cells (FIG. 7D). However strong inhibition was observed with higher concentrations of bufalin (data not shown). By contrast, poly I:C and poly dA:dT induced IFNβ expression was strongly inhibited in Hela and Mg63 cells by 1 uM bufalin (FIG. 7D). Without wishing to be bound by a theory, these differences can be due to differences in the expression levels of the sodium-potassium pump, or the signaling components affected by the drug in different cells lines. The inventors discovered that the expression of the ATP1a1 gene, which encodes the alpha subunit of the sodium pump, is much higher in Hela and MG63 cells than in 293T and Namalwa cells, and the expression of RIG-I and MDA5 followed the same trend (FIG. 7E).

### Example 3

### Bufalin Inhibits RIG-I Activation

The inventors next performed experiments to determine the step at which bufalin blocks the induction of virus-inducible genes. First, the inventors examined the activation of the transcription factors IRF3 and NFκB. Native gel analysis, which detects virus-induced IRF3 dimerization revealed that IRF3 dimer formation was blocked by bufalin (FIG. 2B). In addition, IRF3 nuclear translocation was completely blocked by bufalin treatment (FIG. 2C). A similar observation was made with the p65 subunit of NFκB, where bufalin blocked its nuclear localization in response to virus induction (FIG. 2C). These observations were consistent with reporter assays showing that virus induction of PRDIII/I (activated by IRF3) and PRDII (activated by NFkB) elements were strongly inhibited by bufalin (FIG. 1B). Formation of the IRF3 dimer was also strongly inhibited by bufalin in Namalwa cells (FIG. 7C).

Thus, bufalin appears to act upstream of IRF3 and p65 activation. RIG-I, MAVS and TBK1 are known upstream factors, and over-expression of any of these proteins can activate IFNβ reporter. See for example, Seth, R. B., Sun, L., Ea, C. K. & Chen, Z. J. Identification and characterization of MAVS, a mitochondrial antiviral signaling protein that activates NF-kappaB and IRF 3. Cell 122, 669-82 (2005); Fitzgerald, K. A. et al. IKKepsilon and TBK1 are essential components of the IRF3 signaling pathway. Nat Immunol 4, 491-6 (2003); and Yoneyama, M. et al. The RNA helicase RIG-I has an essential function in double-stranded RNA-induced innate antiviral responses. Nat Immunol 5, 730-7 (2004), content of all of which is herein incorporated by reference. The inventors therefore determined whether bufalin can block IFN expression induced by the over-expression of these proteins. Remarkably, bufalin only modestly inhibited the induction of IFNβ by any of these proteins. Over-expression of MAVS and TBK1 strongly induced the expression of the IFNβ reporter, and the addition of bufalin had no effect on MAVS induction and little effect on TBK1 induction (FIG. 2D). Over-expression of full length RIG-I protein alone appeared to be a less potent activator of IFNβ reporter, but it greatly enhanced the virus induced IFNβ expression. Bufalin treatment only slightly reduced virus induction of the IFNβ reporter when RIG-I was over-expressed (FIG. 2D). These data demonstrate that the target of bufalin in the IFN [symbol] signaling pathway lies upstream from RIG-I. Since RIG-I is the most upstream sensor of SeV induction the inventors considered the possibility that the activation of RIG-I is targeted by bufalin.

### Example 4

### The RIG-I ATPase is Inhibited in Bufalin Treated Cells

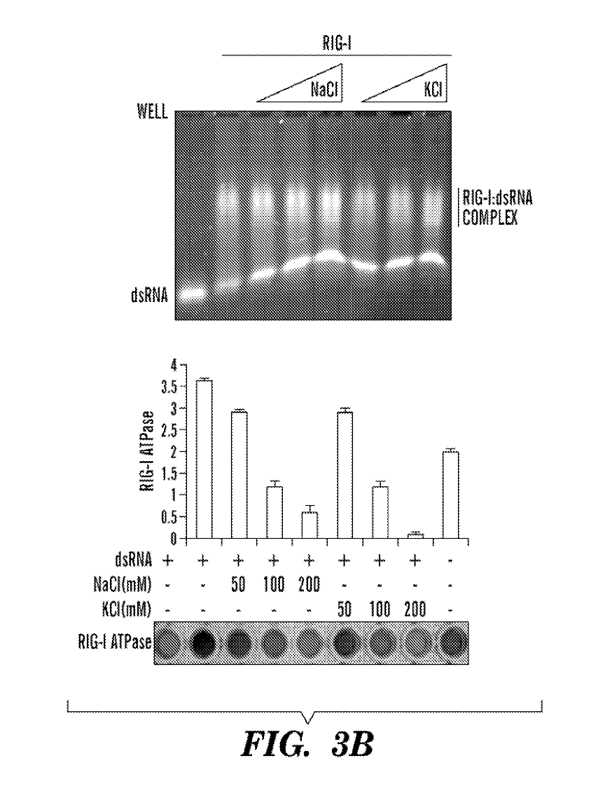

To determine whether the enzymatic activity of the most upstream sensor of virus infection was altered by bufalin treatment, the inventors directly assayed the effects of Bufalin on the activation of RIG-I. First, the inventors examined the RNA binding activity of RIG-I. Since the basal expression level of RIG-I was very low in 293T cells (FIG. 7E), the inventors generated 293T cells stably expressing flag-tagged RIG-I protein. Bufalin treatment also significantly inhibited virus induced IFNβ expression in these cells (FIG. 3A). The inventors then transfected these cells with biotin labeled dsRNA (also bearing a 5′-ppp group, as it was generated by in vitro T7 RNA polymerase transcription) in the presence or absence of bufalin. The associated proteins were captured with NeutrAvidin beads and separated on SDS-PAGE. RNA binding by RIG-I was detected by blotting with anti-RIG-I antibody. In contrast to the strong repression of IFNβ gene induction (FIG. 3A, bottom panel), bufalin treatment did not significantly decrease dsRNA binding by RIG-I (FIG. 3A, top panel).

RIG-I has been shown to undergo dimerization after sensing its ligand, and this dimerization can be studied by native gel electrophoresis. See, Cui, S. et al. The C-terminal regulatory domain is the RNA 5′-triphosphate sensor of RIG-I. Mol Cell 29, 169-79 (2008); Saito, T. et al. Regulation of innate antiviral defenses through a shared repressor domain in RIG-I and LGP2. Proc Natl Acad Sci USA 104, 582-7 (2007); and Malathi, K., Dong, B., Gale, M., Jr. & Silverman, R. H. Small self-RNA generated by RNase L amplifies antiviral innate immunity. Nature 448, 816-9 (2007), content of all of which is herein incorporated by reference. The inventors therefore compared the RIG-I mobility in native gel electrophoresis with/without bufalin treatment. The inventors used both the stable line and Namalwa cells, where RIG-I expression level is relatively higher. No reproducible difference in gel mobility was detected with either cell (data not shown).

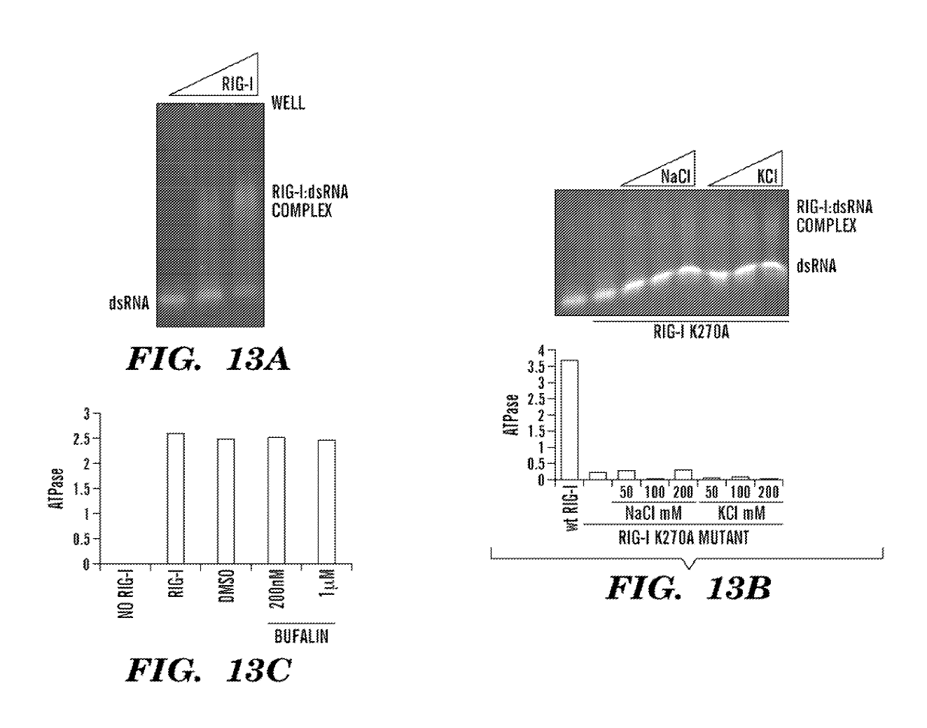

The inventors also designed experiments to test the effects of bufalin on the RNA helicase (ATPase) activity of RIG-I, which is also critical for antiviral signaling. See for example, Myong, S. et al. Cytosolic viral sensor RIG-I is a 5′-triphosphate-dependent translocase on double-stranded RNA. Science 323, 1070-4 (2009) and Saito, T. et al. Regulation of innate antiviral defenses through a shared repressor domain in RIG-I and LGP2. Proc Natl Acad Sci USA 104, 582-7 (2007), content of both of which is herein incorporated by reference. Previous studies have shown that a major effect of bufalin treatment was the elevation of the intracellular sodium concentrations. See, Langer, G. A. Ionic basis of myocardial contractility. Annu Rev Med 28, 13-20 (1977) and Miura, D. S. & Biedert, S. Cellular mechanisms of digitalis action. J Clin Pharmacol 25, 490-500 (1985), content of both of which is herein incorporated by reference. In 293T cells, the intracellular sodium concentration increased rapidly from about 20 mM to over 120 mM with 1 μM bufalin treatment (FIG. 3C). The inventors therefore conducted in vitro experiments to directly test whether the RIG-I helicase activity is affected by different salt concentrations. Flag tagged recombinant RIG-I protein was purified from 293T cells by immunoprecipitation and eluted using a Flag peptide. The protein was incubated with dsRNA probe before addition of ATP and further incubated for 15 min. Free phosphates released from the ATPase hydrolysis were measured with the Biomol Green Reagent. Consistent with published results (Takahasi, K. et al. Nonself RNA-sensing mechanism of RIG-I helicase and activation of antiviral immune responses. Mol Cell 29, 428-40 (2008), addition of dsRNA boosted RIG-I ATPase activity. Strikingly, increasing concentration of both sodium and potassium (from 50 mM to 200 mM) strongly inhibited RIG-I ATPase activity (FIG. 3B, bottom panel), while the dsRNA binding assay of the same samples showed similar binding efficiency by RIG-I under different salt concentrations (FIG. 3B, top panel). The binding of dsRNA increased proportionally to increasing amounts of RIG-I protein, suggesting it is specific for RIG-1 binding (FIG. 13). These observations also agreed with the biotin labeled dsRNA pull down assay where similar binding of transfected dsRNA was detected with/without bufalin treatment (FIG. 3A). Control experiments with a helicase dead mutant RIG-I protein (K270A) (Saito, T. et al. Regulation of innate antiviral defenses through a shared repressor domain in RIG-I and LGP2. Proc Natl Acad Sci USA 104, 582-7 (2007) and Sumpter, R., Jr. et al. Regulating intracellular antiviral defense and permissiveness to hepatitis C virus RNA replication through a cellular RNA helicase, RIG-I. J Virol 79, 2689-99 (2005)) showed similar binding of the protein to dsRNA, while the ATPase activity was extremely low (FIG. 13B). These data demonstrate that the observations with the wild type protein are specific. Without wishing to be bound by a theory, the RIG-I helicase (ATPase) activity is strongly inhibited by bufalin, and as a result the antiviral signaling pathway can be severely impaired. This inhibition is through a bufalin-induced major increase of intracellular sodium concentration, but not bufalin itself (FIG. 13C). As shown in FIG. 3C, in 293T cells, the intracellular sodium concentration increased rapidly from about 20 mM to over 120 mM with 1 μM bufalin treatment.

### Example 5

### Bufalin Inhibits IFNβ Expression Through the Sodium Pump

Cardiac glycosides bind specifically to the sodium pump on the plasma membrane and inhibit its activity. See for example, Prassas, I. & Diamandis, E. P. Novel therapeutic applications of cardiac glycosides. Nat Rev Drug Discov 7, 926-35 (2008). The normal function of the sodium pump is to maintain intracellular ion homeostasis (at the expense of ATP hydrolysis, it pumps out three sodium ions as it pumps in two potassium ions during each cycle). The direct effect of inhibiting the sodium pump by cardiac glycosides is to change intracellular ion concentrations. For example, sodium and calcium concentrations are elevated and the potassium concentration decreases (Langer, G. A. Ionic basis of myocardial contractility. Annu Rev Med 28, 13-20 (1977)). It is the increased concentration of sodium but not the decreased potassium that directly leads to the inhibition of the ATPase activity of RIG-I (FIG. 3B).

The sodium pump is composed of the catalytic alpha subunit and the structural beta subunit, both are encoded by four genes in most species, see for example, Morth, J. P. et al. Crystal structure of the sodium-potassium pump. Nature 450, 1043-9 (2007) and Shinoda, T., Ogawa, H., Cornelius, F. & Toyoshima, C. Crystal structure of the sodium-potassium pump at 2.4 A resolution. Nature 459, 446-50 (2009), content of both of which is herein incorporated by reference. Different isoforms of the alpha and beta subunits display tissue specific expression pattern (James, P. F. et al. Identification of a specific role for the Na,K-ATPase alpha 2 isoform as a regulator of calcium in the heart. Mol Cell 3, 555-63 (1999)), with the ATP1a1 most widely expressed. The conserved aspartic acid (Asp) 376 in the active center of the alpha subunit is continuously phosphorylated and dephosphorylated during pumping activities (Ohtsubo, M., Noguchi, S., Takeda, K., Morohashi, M. & Kawamura, M. Site-directed mutagenesis of Asp-376, the catalytic phosphorylation site, and Lys-507, the putative ATP-binding site, of the alpha-subunit of Torpedo californica Na+/K(+)-ATPase. Biochim Biophys Acta 1021, 157-60 (1990)).

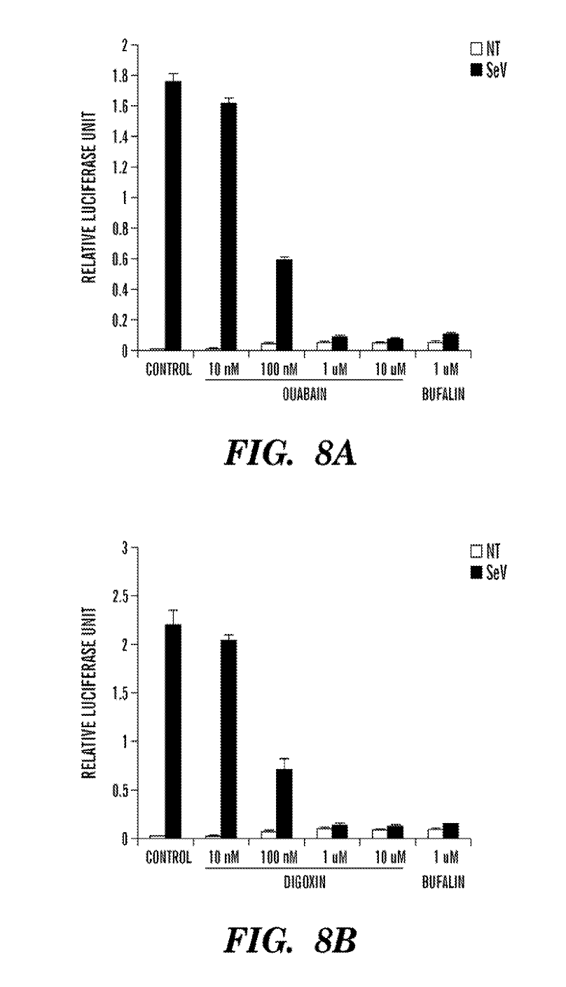

To show that inhibition of IFN [symbol] expression by bufalin is exclusively through the sodium pump, the inventors first tested whether other cardiac glycosides also inhibit viral induced IFNβ expression. Ouabain and digoxin, which are also cardiac glycosides, and clinically approved to treat congestive heart failure (Prassas, I. & Diamandis, E. P. Novel therapeutic applications of cardiac glycosides. Nat Rev Drug Discov 7, 926-35 (2008)), were as potent as bufalin in inhibiting IFN [symbol] induction, and this inhibition was also dose dependent (FIG. 8). The calculated IC₅₀ for ouabain and digoxin are 64.8 nM and 56.6 nM respectively, relatively higher that that of bufalin (4.3 nM).

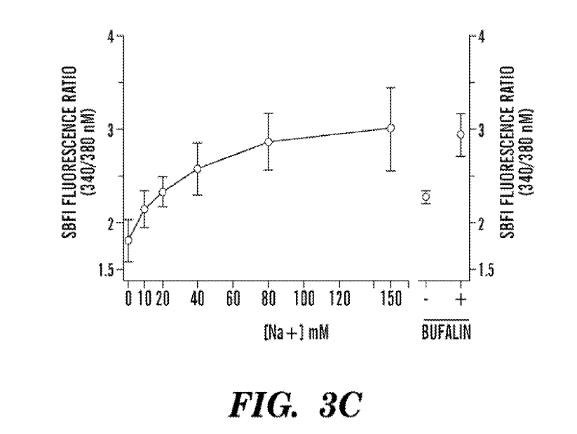

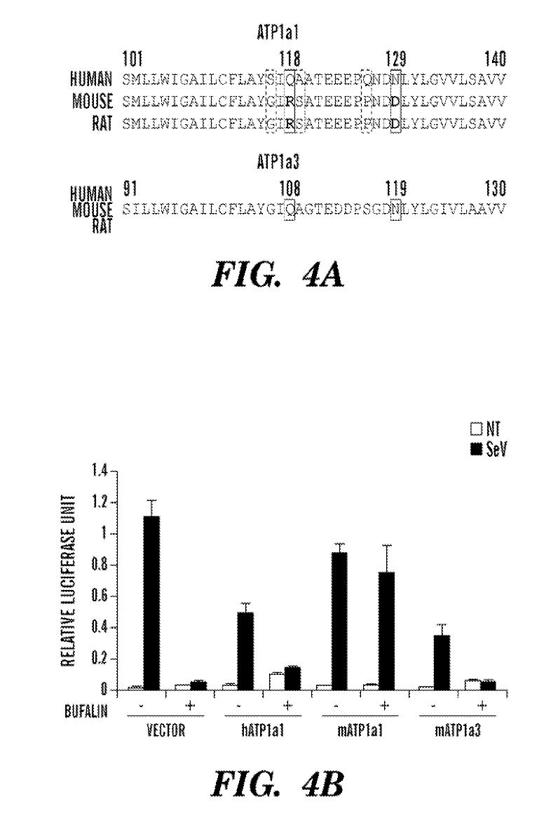

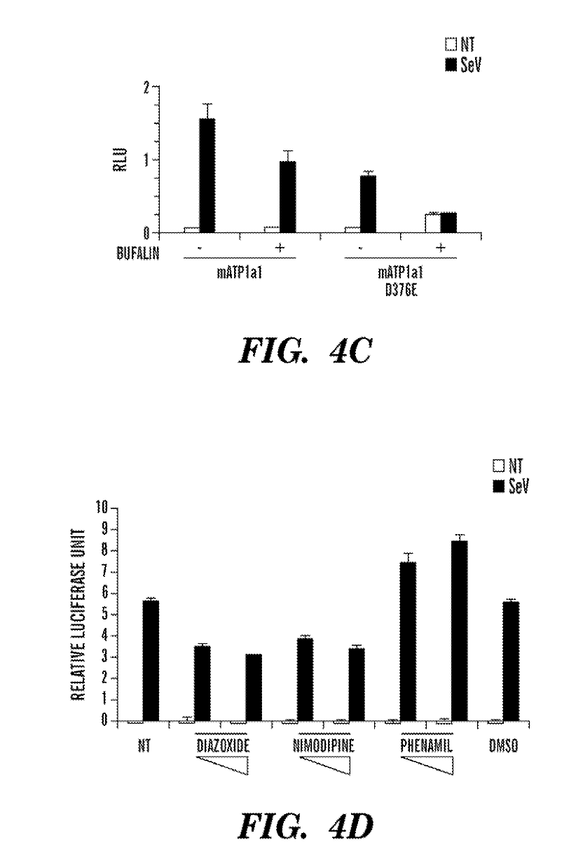

The inventors also performed rescue experiments with the mouse ATP1a1 gene (Simpson, C. D. et al. Inhibition of the sodium potassium adenosine triphosphatase pump sensitizes cancer cells to anoikis and prevents distant tumor formation. Cancer Res 69, 2739-47 (2009)), which was not sensitive to bufalin (FIG. 4A) due to the natural occurring Q118R and N129D single amino acid substitutions (Lingrel, J. B. The physiological significance of the cardiotonic steroid/ouabain-binding site of the Na,K-ATPase. Annu Rev Physiol 72, 395-412). In contrast, the ATP1a3 gene is highly conserved between human and mouse, and sensitive to the drug. The inventors transfected plasmids encoding mouse ATP1a1 and ATP1a3 genes in parallel with human ATP1a1 gene, and infected the cells with virus in the presence or absence of bufalin. The inventors discovered that only the mouse ATP1a1 transfection relieved the inhibition of IFNβ expression by bufalin, while little if any rescue was observed with the drug sensitive human ATPla 1 and mouse ATP1a3 proteins (FIG. 4B). These data demonstrate that bufalin inhibits IFNβ gene activation entirely through its binding and inhibition of the sodium pump.

To test whether the mouse ATP1a1 protein can rescue bufalin inhibition of IFN-beta induction independently of its catalytic activity, the inventors generated a catalytic inactive mutant (D376E) mouse ATP1a1 expression construct, and it failed to rescue the bufalin inhibited IFN [symbol] induction by virus (FIG. 4C). These data demonstrate that the enzymatic activity of the sodium pump is required for the optimal induction of IFN gene, presumably to maintain the appropriate concentrations of intracellular ions.

### Example 6

### Intracellular Ion Concentrations Modulate IFNβ Expression

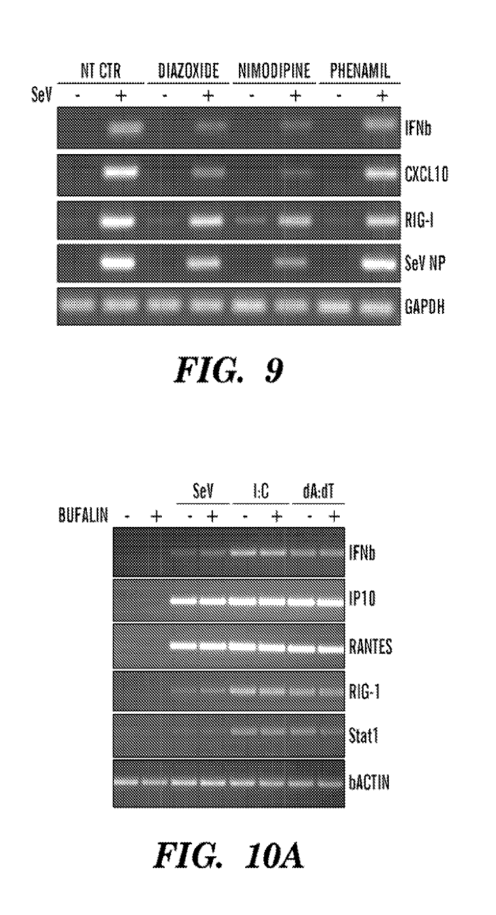

Since the major function of the pump is to maintain the cellular ion homeostasis, the inventors reasoned that it might be possible to inhibit or stimulate IFNβ induction by varying the intracellular ion concentration. The inventors thus performed luciferase reporter assays with an ion-channel ligand library, which contains various modulators for sodium, potassium, calcium and chloride channels. The experiments were conducted similarly to the initial screening assays: 293T cells were transfected with reporter plasmid, then chemical ligands were added before starting virus infection, and luciferase activity measured after another 24 hrs. A few ligands showed considerable negative effects on IFNβ induction, among these were Nimodipine, a dihydropyridine-type voltage-sensitive (L-type) calcium channel blocker (Izquierdo, I. Nimodipine and the recovery of memory. Trends Pharmacol Sci 11, 309-10 (1990)), and Diazoxide, a selective opener of ATP sensitive potassium channel (Trube, G., Rorsman, P. & Ohno-Shosaku, T. Opposite effects of tolbutamide and diazoxide on the ATP-dependent K+ channel in mouse pancreatic beta-cells. Pflugers Arch 407, 493-9 (1986)). As shown in FIG. 4D, more inhibition of the IFNβ reporter was achieved with increasing concentrations of both nimodipine and diazoxide. The virus induced expression of the endogenous IFNβ gene was similarly inhibited by nimodipine and diazoxide in 293T cells (FIG. 9). These data demonstrate that not only sodium, but potassium and calcium can also modulate cellular antiviral responses. Interestingly, one type of amiloride-sensitive sodium channel inhibitor, phenamil (Garvin, J. L., Simon, S. A., Cragoe, E. J., Jr. & Mandel, L. J. Phenamil: an irreversible inhibitor of sodium channels in the toad urinary bladder. J Membr Biol 87, 45-54 (1985)) consistently enhanced the activation of IFNβ gene expression in reporter assays (FIG. 4D). However, the stimulatory effect on endogenous IFNβ gene induction was less potent (FIG. 9).

### Example 7

### Knockingdown Pump Expression Suppresses IFNβ Induction

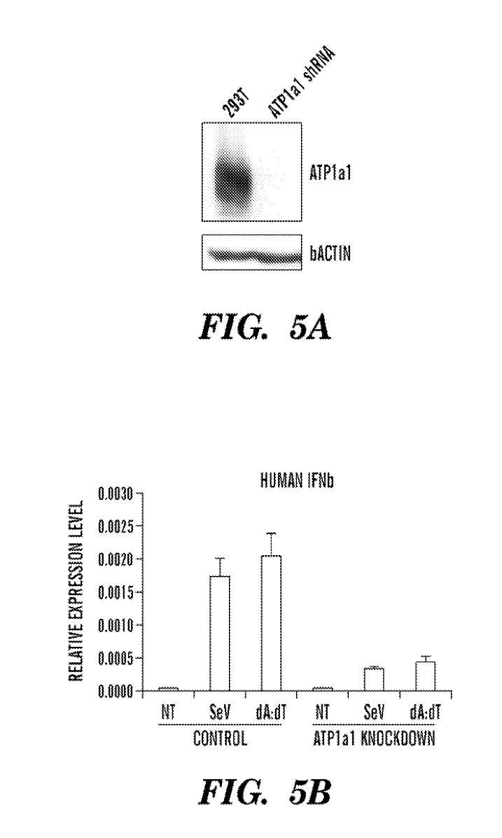

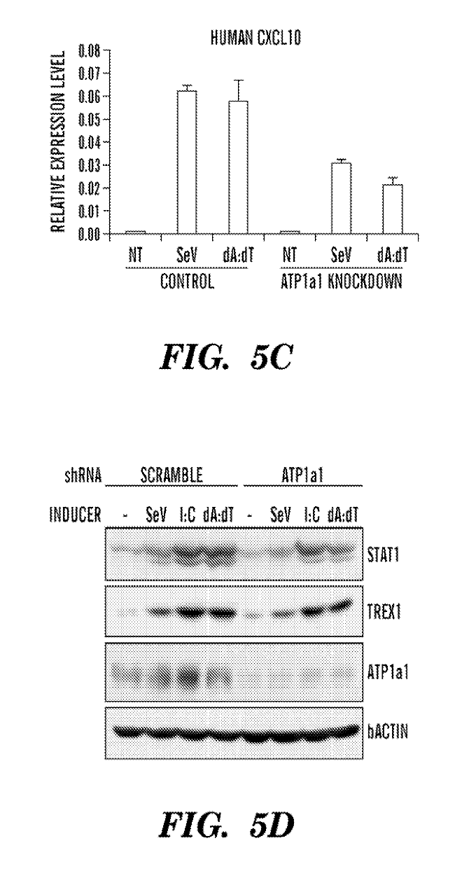

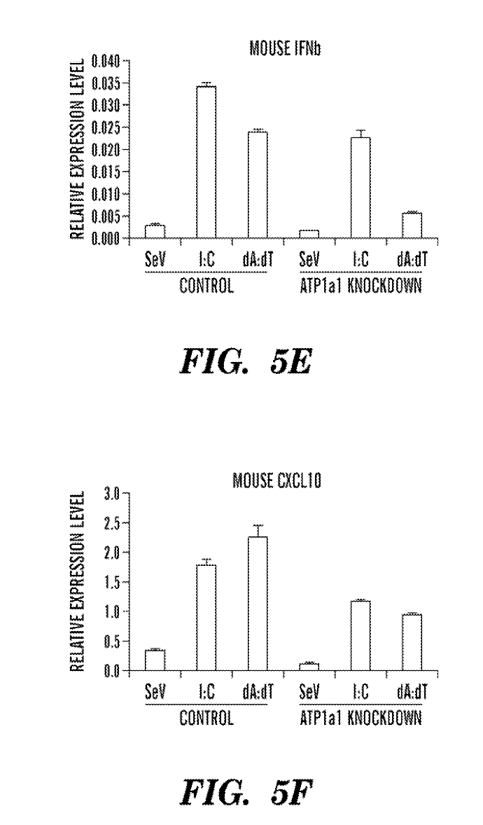

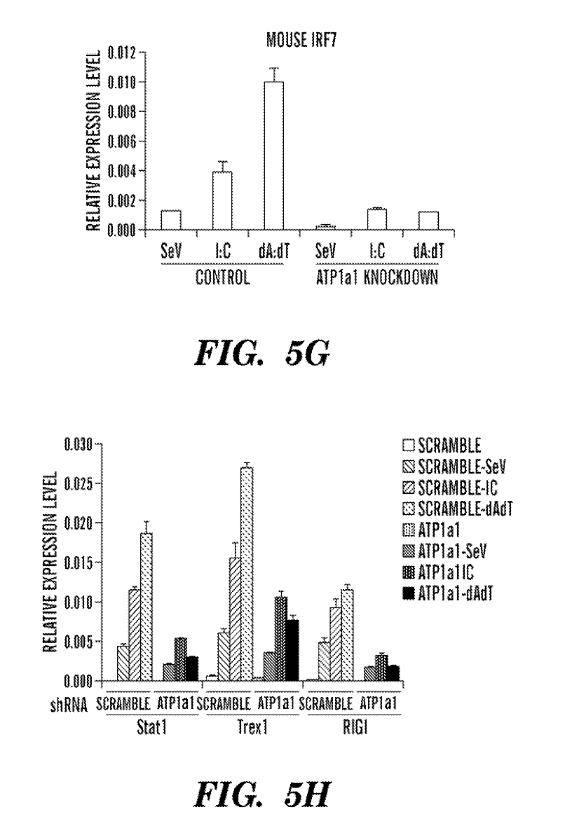

To further examine the role of the sodium pump in IFNβ gene expression, the inventors conducted shRNA knockdown experiments. The inventors first carried out the knockdown experiments in 293T cells, where the induction of IFNβ and Cxcl10 genes by virus and dsDNA were greatly inhibited when ATP1a1 gene expression was knocked down by shRNA specifically targeting the gene (FIGS. 5A-5C). Real time PCR quantification showed about 4 fold reduction of IFNβ (FIG. 5B), and at least 2 fold reduction of CXCL10 gene (FIG. 5C) in knockdown cells compared to control cells for both inducers.

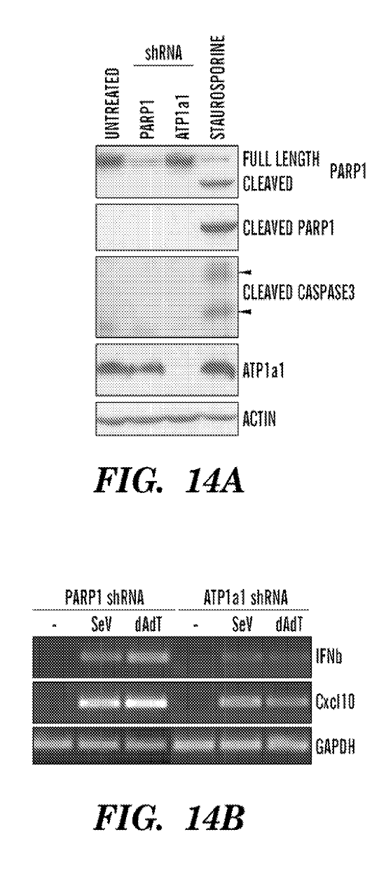

The impaired IFN-beta induction in ATP1a1 knock-down cells was not due to apoptosis (FIG. 14A) and was similarly observed when compared to unrelated PARP1 knock-down cells (FIG. 14B). Total protein lysates from 293T cells were separated on SDS-PAGE. Cells were either untreated, infected with lentivirus to specifically knock-down the expression of PARP1 or ATP1a1, or treated with sturosporine (4 μM for 8 hours) to induce apoptosis. The expression of PARP1, cleaved PARAP1, cleaved Caspase3, ATP1a1 and beta-actin were analyzed by Western blot (FIG. 14A).

293 T cells with PARP1 or ATP1a1 knocked-down were subjected to SeV infection or dsDNA (poly dA:dT) transfection. Total RNA was extracted after 6 hours and the expression of IFN-beta, Cxcl10, and GAPDH was analyzed by RT-PCR. As shown in FIG. 14B, the induction of IFN-beta and Cxcl10 was impaired in ATP1a1 knock-down cells.

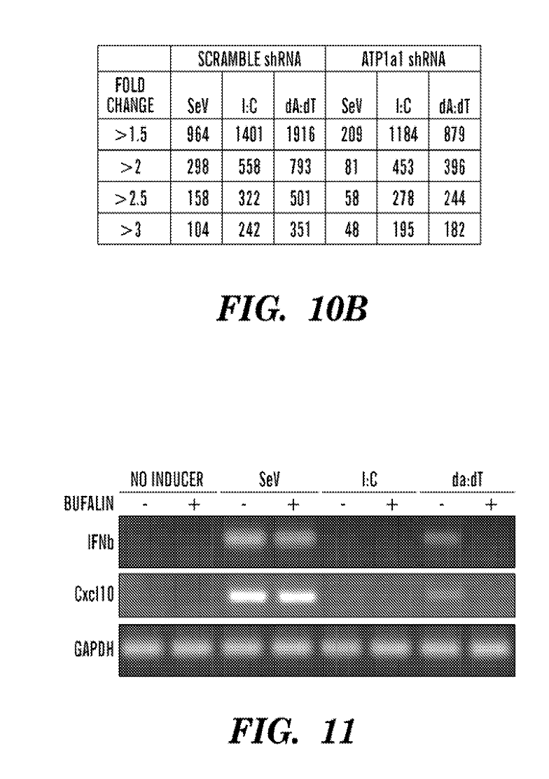

As discussed above, the mouse ATP1a1 protein is not sensitive to cardiac glycoside binding. Not surprisingly, the inventors found little effects of bufalin on the induction of IFNβ gene by various inducers in mouse embryonic fibroblasts (MEFs), which express only the ATP1a1 gene out of the four alpha subunits (FIG. 10A). Interestingly, induction of IFNβ and CXCL10 genes and other ISGs (RIG-I, IRF7, Trex1, STAT1 etc) were significantly reduced (>30% for IFNβ and CXCL10 genes, and at least 50% for most other genes) at both mRNA and protein levels when the expression level of the ATP1a1 gene was reduced by shRNA (FIG. 5D-H). Genome wide analysis revealed that not only was the expression level of target genes affected, but the number of genes induced by the inducers tested (SeV, poly I:C, and poly dA:dT) were significantly affected (FIG. 10B).

Taken together the data demonstrate that normal sodium pump activity is critically involved in the optimal induction of the IFNb gene after pathogen challenge. The main mechanism is through balanced intracellular ion concentration. RIG-I, a key component of the virus activated signaling pathway, is sensitive to the intracellular ion fluctuations. Without wishing to be bound by a theory, these findings demonstrate that manipulating intracellular ion concentration effectively modulate cellular antiviral signaling pathway. Since mis-regulation of cytokine production has been implicated in many human diseases, ion concentration modulators can serve as promising agents to treat these diseases.

### Example 8

### Bufalin Inhibits TNF Signaling

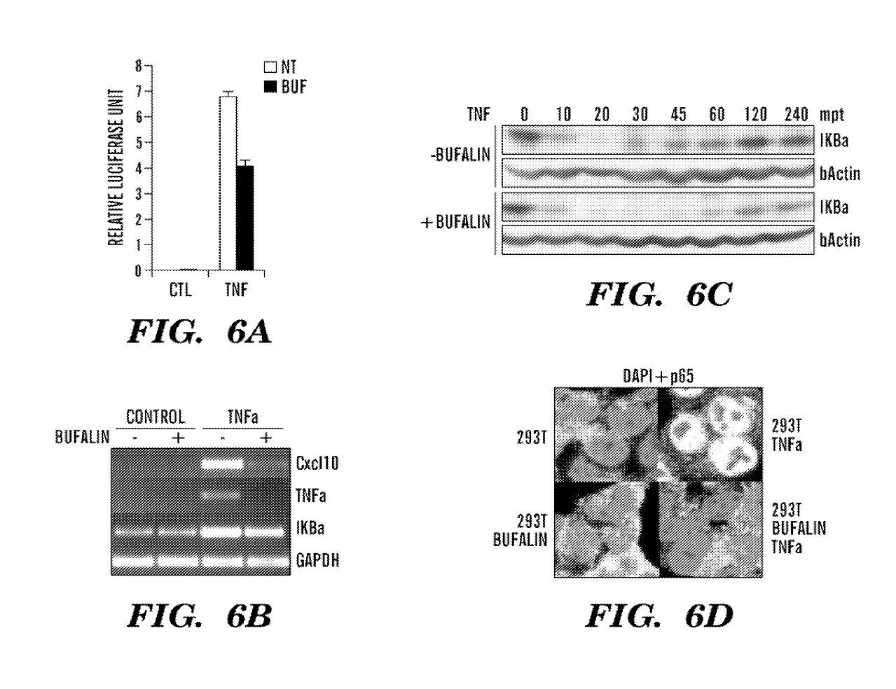

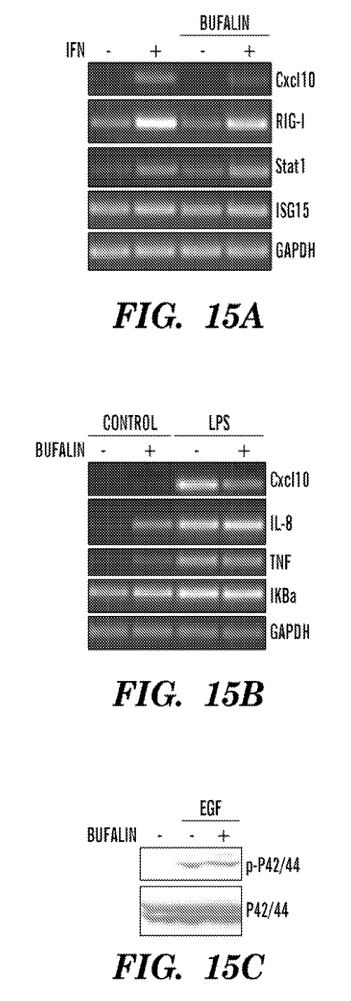

Since sodium pump maintains physiological intracellular ion concentrations, any signaling event sensitive to changes in ion concentrations could be affected by cardiac glycosides. The inventors therefore carried out experiments to test the effects of bufalin on other signaling pathways. Specifically, the inventors tested interferon (IFN), tumor necrosis factor (TNF), epidermal growth factor (EGF) signaling, and treatment with lipopolysacchoride (LPS). While effects of bufalin on IFN, EGF and LPS were selective and generally weaker (FIG. 15), the effects on TNF signaling were strong. The latter inhibition was demonstrated using a luciferase reporter assay with the PRDII element (NFkB binding site) driving luciferase gene expression. Treatment with TNF induced the expression of the reporter gene. As shown in FIG. 6A the expression of the luciferase gene decreased by 40% after bufalin treatment. In addition, bufalin significantly inhibited the expression of endogenous TNF target genes (FIG. 6B).

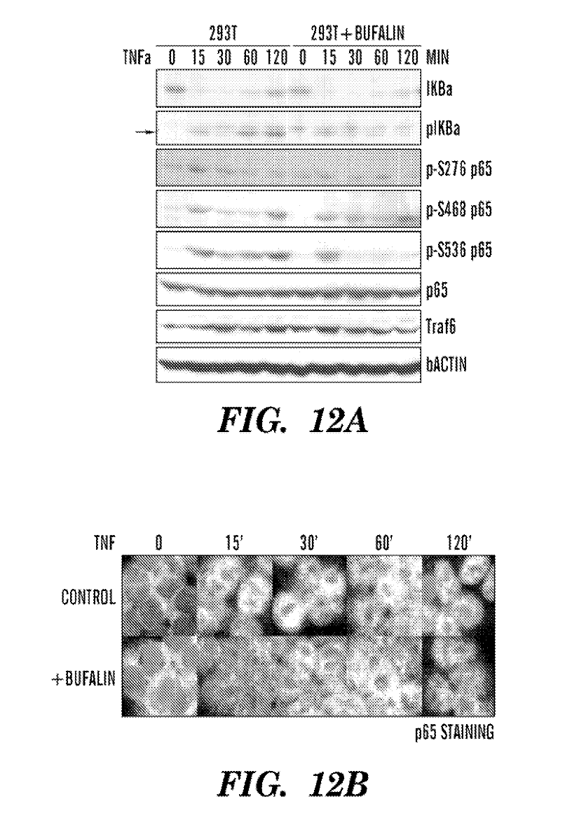

To investigate the mechanisms of the inhibitory effects of bufalin on TNF signaling, the inventors monitored the degradation of IKBα, protein in the presence or absence of bufalin. Bufalin did not affect the initial degradation of IKBα, as the degradation of IKBα was almost complete after 20 min of TNFa treatment in the presence or absence of bufalin. However the subsequent re-synthesis of IKBα was significantly delayed and the level reduced in cells treated with bufalin (FIG. 6C). The level of IKBα was at least twice as high in the control sample after 45-60 min compared to the bufalin treated sample. Monitoring the serine 32/36 phosphorylation of IKBα revealed similar levels phospho-IKBα after 15-20 minutes of TNF induction in both bufalin treated and non-treated cells. By contrast, the level of IKBα phosphorylation (S32/36) was significantly higher 1 hr after TNF stimulation in control cells compared to bufalin treated cells (FIG. 12A). The inventots also discovered that bufalin strongly inhibited the nuclear translocation of the NFκB p65 subunit after TNF stimulation. This effect was greater in the early time points: 15-20 minutes after TNF stimulation, p65 was in the nucleus in all control cells, while virtually no p65 was observed in the nucleus of the bufalin treated cells (FIG. 6D). Subsequently, a gradual increase in nuclear p65 was observed (FIG. 12B). These date demonstrate that the nuclear translocation of p65 is sensitive to bufalin treatment, although the initial degradation of IKBα is not.

### Example 9

### Bufalin does not Induce Apoptosis or Autophagy in 293T Cells

293T cells were either untreated, or treated with increasing amounts of bufalin (1 nM to 10 μM) for 8 hours and subjected to either CellTiter-Blue viability assay (Promega) or to CellTiter-Glo Luminescent viability assay (Promega). As shown in FIGS. 16A and 16B, Bufalin did not severely impair cell viability in 293T cells.

For flow cytometry analysis, 293T cells were treated with bufalin (1 μM for 8 hours), staurosporine (4 μM for 4 hours), DMSO or left untreated. Cells were harvested and washed, then stained with Allophycocyanin (APC) conjugated Annexin V and 7-AAD for 15 minutes and subjected to flow cytometry analysis. Results are shown in FIG. 16C. Percentile of Annexin V or 7-AAD positive populations were 2.62 for untreated, 2.84 for DMSO, 1.95 for bufalin, and 17. 11 for sturosporine.

Apoptosis and autophagy were also analyzed by Western blots. 293T cells were treated with increasing amounts of bufalin (1 nM to 10 μM) staurosporine (4 μM) or bafilomycin A1 (BFA, 100 nM) for 8 hours. Total protein lysates were prepared and separated on SDS-PAGE for Western blot analysis of PARP1, cleaved PARP1, cleaved Caspase3, LC3B, ATP1a1, and beta-actin expression. As shown in FIG. 16D, Bufalin treatment did not induce apoptosis or autophagy in 293T cells. Cleavage products of PARP1 and Caspase3 (an indication of apoptosis, induced by staurosporine), and the strong induction of LC3B-II (a marker of autophagy, induced by both staurosporine and BFA) were also not observed in bufalin treated cells (FIG. 16D).

### Discussion

The inventors have discovered, inter alia, that cardiac glycosides are potent inhibitors of IFNβ gene activation by virus, dsRNA, and dsDNA. Although two recent studies suggested that cardiac glycosides can induce cellular signaling events independent of their inhibition of the sodium pump (Prassas, I. & Diamandis, E. P. Novel therapeutic applications of cardiac glucosides. Nat. Rev. Drug Disc. 7, 926-935 (2008) and Xie, Z. & Cai, T. Na+-K+−-ATPase-mediated signal transduction: from protein interaction to cellular function. Mol Interv 3, 157-68 (2003), content of both of which is herein incorporated by reference in its entirety), the inventors discovered that inhibition of IFNβ expression by cardiac glycosides was exclusively through blocking the activities of the sodium pump rather than an off-target effect of the drug (FIG. 4B, C). The inventors have also demonstrated that bufalin inhibits the activation of both IRF-3 and NF-κB, transcription factors required for IFNβ gene expression. Overexpression experiments with intermediates in the virus-induction signaling pathway revealed that bufalin acts on an early step of the pathway, with the inhibition of the ATPase activity of the RNA sensor RIG-I as a primary mechanism. Without wishing to be bound by a theory, this inhibition appeared to be the consequence of the ability of bufalin to change the intracellular ion concentration by inhibiting the sodium pump. The inventors also provide evidence that the helicase (ATPase) activity of the RNA sensor RIG-I can be the target for high salt inhibition of the signaling pathway. None of the downstream signaling components were seen to be directly affected by bufalin, and the in vitro ATPase activity of RIG-I was sensitive to increasing concentrations of salt.

Furthermore, RIG-I can also be the target in the dsDNA activation pathway. Two recent studies showed that AT-rich dsDNA can signal through RIG-I to activate IFN gene. This was achieved through a critical sensor: RNA polymerase III, which transcribes 5′-ppp bearing “panhandle” RNA from the dsDNA template, these nascent RNAs then activated the IFNb expression through the RIG-1-MAVS pathway. See, for example, Ablasser, A. et al. RIG-I-dependent sensing of poly(dA:dT) through the induction of an RNA polymerase III-transcribed RNA intermediate. Nat Immunol 10, 1065-72 (2009) and Chiu, Y. H., Macmillan, J. B. & Chen, Z. J. RNA polymerase III detects cytosolic DNA and induces type I interferons through the RIG-I pathway. Cell 138, 576-91 (2009), content of both of which is herein incorporated by reference. The inventors discovered that the activity of cytoplasmic RNA polymerase III is also inhibited by bufalin (FIG. 11). When total RNA from dsDNA transfected cells treated with bufalin was transfected into new cells, it failed to induce the expression of IFN-beta gene (FIG. 11).

The inventors further discovered that the RNA binding and helicase activities of RIG-I were separable, as demonstrated by in vitro RNA binding and ATPase assays (FIG. 3B). Cardiac glycosides inhibited the function of the sodium pump, and subsequently lead to elevated intracellular sodium and calcium concentrations. Although the RNA binding of RIG-I was not affected by bufalin treatment (FIG. 3A), its ATPase activity was inhibited by bufalin induced higher salt concentrations. This agrees with a previous report showing the ATPase of the recombinant Helicase domain of RIG-I is inhibited by higher concentration of sodium chloride. See Gee, P. et al. Essential role of the N-terminal domain in the regulation of RIG-I ATPase activity. J Biol Chem 283, 9488-96 (2008), content of which is herein incorporated nu reference.

It is interesting to note that the activity of some DNA dependent helicases is also sensitive to higher salt concentration, such as E. Coli REP and UvrD proteins. See for example, Lohman, T. M., Chao, K., Green, J. M., Sage, S. & Runyon, G. T. Large-scale purification and characterization of the Escherichia coli rep gene product. J Biol Chem 264, 10139-47 (1989) and Runyon, G. T., Wong, I. & Lohman, T. M. Overexpression, purification, DNA binding, and dimerization of the Escherichia coli uvrD gene product (helicase II). Biochemistry 32, 602-12 (1993), content of both of which is herein incorporated by reference.

Without wishing to be bound by theory, there are conserved residues in the helicase domain which are sensitive to ion concentrations. A recent study found that RIG-I also possesses helicase dependent translocase activity, which is required to shuttle RIG-I repeatedly along the dsRNA ligand. See Myong, S. et al. Cytosolic viral sensor RIG-I is a 5′-triphosphate-dependent translocase on double-stranded RNA. Science 323, 1070-4 (2009), content of which is herein incorporated by reference. The role of this translocase activity in signal transduction is not understood. Without wishing to be bound by a theory, this activity may be required for the unwinding of the RNA and the binding of RIG-I to the MAVS protein on the mitochondrial membrane.

Alternatively, cardiac glycosides can inhibit IFNβ expression by blocking viral replication. However, the inventors have shown that cardiac glycosides also strongly inhibited the activation of the IFN-β gene by dsRNA transfection, demonstrating that viral replication was not the major reason for the inhibition of IFNβ expression, since transfected dsRNA activates IFNβ expression independent of replication. Virus replication was inhibited by bufalin at longer incubation times. A previous study has described inhibition of influenza virus replication by cardiac glycosides treatment. See, Hoffmann, H. H., Palese, P. & Shaw, M. L. Modulation of influenza virus replication by alteration of sodium ion transport and protein kinase C activity. Antiviral Res 80, 124-34 (2008), content of which is herein incorporated by reference. However, this does not explain the inhibition of IFN-β gene expression as discovered by the inventors.

The activity of cytoplasmic RNA polymerase III was also inhibited by bufalin. When total RNA from dsDNA transfected cells with/without bufalin treatment was extracted, and re-transfected into new cells, the ability of these RNAs to induce IFNβ inducing ability of these RNA depends upon whether the cells were treated with bufalin. RNA from the bufalin treated sample failed to induce the expression of IFNβ gene (FIG. 11).

The inventors have also demonstrated that bufalin inhibits the activation of NFκB by TNF. Remarkably, bufalin inhibited the nuclear translocation of NF-κB in response to TNF, but not the degradation of IKBα. The nuclear translocation of NF-κB requires importin alpha 3 and 4, and post-translational modifications like phosphorylation or sumoylation can regulate the nuclear translocation of NFkB. See for example, Fagerlund, R., Kinnunen, L., Kohler, M., Julkunen, I. & Melen, K. NF-{kappa}B is transported into the nucleus by importin {alpha}3 and importin {alpha}4. J Biol Chem 280, 15942-51 (2005); Drier, E. A., Huang, L. H. & Steward, R. Nuclear import of the Drosophila Rel protein Dorsal is regulated by phosphorylation. Genes Dev 13, 556-68 (1999); Bhaskar, V., Valentine, S. A. & Courey, A. J. A functional interaction between dorsal and components of the Smt3 conjugation machinery. J Biol Chem 275, 4033-40 (2000); and Cyert, M. S. Regulation of nuclear localization during signaling. J Biol Chem 276, 20805-8 (2001), content of all of which is herein incorporated by reference. The inventors examined the effects of bufalin on the phosphorylation of p65, and found little difference on the initial phosphorylation of S276, S468 and S536 residues between bufalin treated and non-treated samples. However, S536 phosphorylation appeared weaker in later time points after bufalin treatment (FIG. 12A). This may explain reduced NFκB transactivation activity in bufalin treated cells, but does not explain the strong cytoplasm retention of p65 after 15-20 minutes after TNF stimulation. Accordingly, without wishing to be bound by a theory, other factors involved in p65 nuclear translocation must be affected by bufalin treatment. It is interesting that another cardiac glycoside, digitoxin, has been shown to inhibit TNF signaling by blocking the recruitment of TRADD of the TNF receptor, and directly inhibiting IKBα degradation (Yang, et al., Cardiac glycosides inhibit TNF-alpha/NF-kappaB signaling by blocking recruitment of TNF receptor-associated death domin to the TNF receptor. Proc. Natl. Acad. Sci. USA 102, 9631-9636 (2005). By contrast, as discussed above, the inventors demonstrated that IKBα degradation is not inhibited by bufalin.

### Example 10

### Inhibition of Lipopolysaccharide (LPS) Induced Lethality in Mice

The inventors carried out animal experiments with cardiac glycosides (CGs), to test whether CG can reduce the adverse effects caused by cytokine overproduction. Inventors obtained a specific knock-in mouse strain from Dr. Jerry Lingrel at the University of Cincinnati. The mouse gene encoding the alpha 1 subunit (ATP1a1) of the sodium pump (Na—K ATPase) contains several point mutations compared to its human counterpart, which makes mouse cells much less sensitive to CG treatment. Dr. Lingrel has specifically engineered this mouse strain with a human version of the ATP1a1 replacing the endogenous mouse gene. This “humanized” strain of mice is much more sensitive to CG treatment, mimicking the human situation. With this strain, inventors have established the conditions to administer a safe and effective dose of bufalin into mice.

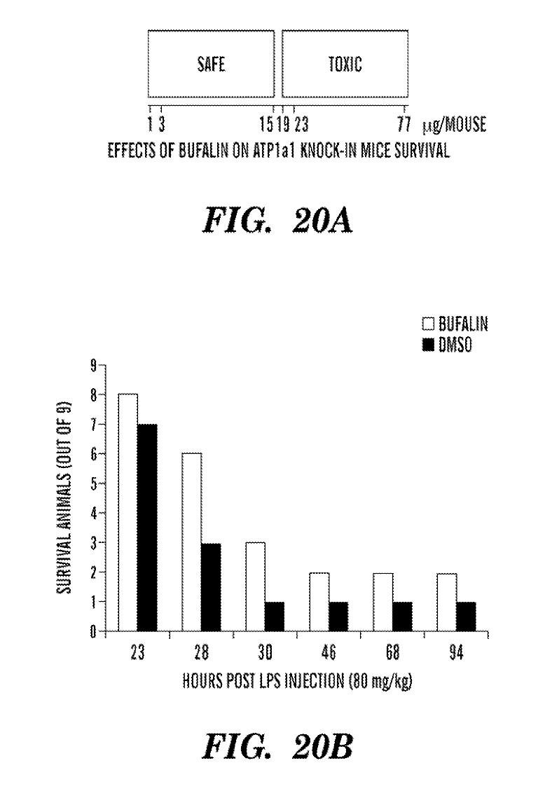

As summarized in FIG. 20A, less than 5 μg of bufalin/mouse (˜10 weeks age, ˜30 g) of intraperitoneal injection is safe, and this injection can be repeated daily for at least a week. A dose greater than 19 μg/mouse appears to be toxic, resulting in animal inactivity, weight loss, signs of sickness and even death under high doses.

Mice (10 weeks old, 9 for each group) were injected intraperitoneally with either bufalin (15 μg/mouse, dissolved in DMSO, and diluted with PBS to make final solution 2% DMSO) or with PBS (containing 2% DMSO), followed by a high dose of LPS injection (i.p., 80 mg/kg) after 30 min. The survival of injected animals was closely monitored for 4 days.

While, bufalin is the most potent cardiac glycoside to inhibit interferon production as shown in this study, bufalin can partially reduce the severe lethality induced by a high dose of LPS (80 mg/kg) injections (FIG. 20B). These results show that, cardiac glucosides can be used for treating pathogenic or non-pathogenic infection in vivo. The pathogenic or non-pathogenic infection can be one which can lead to LPS induce shock.

The skilled artisan recognizes that the dose used to treat heart disease is significantly lower than that required to inhibit IFN gene expression, and a subject may not tolerate the dose necessary for interferon-beta inhibition. However, in a recent clinical trial, Chinese traditional medicine huachansu (bufalin is the major component) was used for treating cancer. See, Meng, Z. et al. Pilot study of huachansu in patients with hepatocellular carcinoma, nonsmall-cell lung cancer, or pancreatic cancer. Cancer 115, 5309-18 (2009). The trial demonstrated that up to 9 nM of bufalin was well tolerated in patients. The IC₅₀ for bufalin necessary to inhibit IFNβ expression was found to be 4.3 nM (FIG. 1A), which is less than the well tolerated 9 nM dose described by Meng et al. Thus, the dose of cardiac glycosides necessary to inhibit IFNβ expression is well tolerated by the subject.

### REFERENCES

1. Sen, G. C. Viruses and interferons. Annu Rev Microbiol 55, 255-81 (2001).
2. Garcia-Sastre, A. & Biron, C. A. Type 1 interferons and the virus-host relationship: a lesson in detente. Science 312, 879-82 (2006).
3. Le Bon, A. et al. Type i interferons potently enhance humoral immunity and can promote isotype switching by stimulating dendritic cells in vivo. Immunity 14, 461-70 (2001).
4. Le Bon, A. & Tough, D. F. Links between innate and adaptive immunity via type I interferon. Curr Opin Immunol 14, 432-6 (2002).
5. Banchereau, J. & Pascual, V. Type I interferon in systemic lupus erythematosus and other autoimmune diseases. Immunity 25, 383-92 (2006).
6. Yoshida, H., Okabe, Y., Kawane, K., Fukuyama, H. & Nagata, S. Lethal anemia caused by interferon-beta produced in mouse embryos carrying undigested DNA. Nat Immunol 6, 49-56 (2005).
7. Yarilina, A. & Ivashkiv, L. B. Type I Interferon: A New Player in TNF Signaling. Curr Dir Autoimmun 11, 94-104.
8. Mandl, J. N. et al. Divergent TLR7 and TLR9 signaling and type I interferon production distinguish pathogenic and nonpathogenic AIDS virus infections. Nat Med 14, 1077-87 (2008).
9. Whittemore, L. A. & Maniatis, T. Postinduction turnoff of beta-interferon gene expression. Mol Cell Biol 10, 1329-37 (1990).
10. Raj, N. B., Cheung, S. C., Rosztoczy, I. & Pitha, P. M. Mouse genotype affects inducible expression of cytokine genes. J Immunol 148, 1934-40 (1992).
11. Pandos, M., Shimonaski, G. & Came, P. E. Interferon in mice acutely infected with M-P virus. J Gen Virol 13, 163-5 (1971).
12. Maniatis, T. et al. Structure and function of the interferon-beta enhanceosome. Cold Spring Harb Symp Quant Biol 63, 609-20 (1998).
13. Kawai, T. & Akira, S. Innate immune recognition of viral infection. Nat Immunol 7, 131-7 (2006).
14. Akira, S., Uematsu, S. & Takeuchi, O. Pathogen recognition and innate immunity. Cell 124, 783-801 (2006).
15. Honda, K., Takaoka, A. & Taniguchi, T. Type I interferon [corrected] gene induction by the interferon regulatory factor family of transcription factors. Immunity 25, 349-60 (2006).
16. Sun, L., Liu, S. & Chen, Z. J. SnapShot: pathways of antiviral innate immunity. Cell 140, 436-436 e2.
17. Ford, E. & Thanos, D. The transcriptional code of human IFN-beta gene expression. Biochim Biophys Acta 1799, 328-336.
18. Yoneyama, M. & Fujita, T. Structural mechanism of RNA recognition by the RIG-1-like receptors. Immunity 29, 178-81 (2008).
19. Kato, H. et al. Differential roles of MDA5 and RIG-I helicases in the recognition of RNA viruses. Nature 441, 101-5 (2006).
20. Cui, S. et al. The C-terminal regulatory domain is the RNA 5′-triphosphate sensor of RIG-I. Mol Cell 29, 169-79 (2008).
21. Takahasi, K. et al. Nonself RNA-sensing mechanism of RIG-I helicase and activation of antiviral immune responses. Mol Cell 29, 428-40 (2008).
22. Seth, R. B., Sun, L., Ea, C. K. & Chen, Z. J. Identification and characterization of MAVS, a mitochondrial antiviral signaling protein that activates NF-kappaB and IRF 3. Cell 122, 669-82 (2005).
23. Kawai, T. et al. IPS-1, an adaptor triggering RIG-1- and Mda5-mediated type I interferon induction. Nat Immunol 6, 981-8 (2005).
24. Xu, L. G. et al. VISA is an adapter protein required for virus-triggered IFN-beta signaling. Mol Cell 19, 727-40 (2005).
25. Meylan, E. et al. Cardif is an adaptor protein in the RIG-I antiviral pathway and is targeted by hepatitis C virus. Nature 437, 1167-72 (2005).
26. Tang, E. D. & Wang, C. Y. MAVS self-association mediates antiviral innate immune signaling. J Virol 83, 3420-8 (2009).
27. Baril, M., Racine, M. E., Penin, F. & Lamarre, D. MAVS dimer is a crucial signaling component of innate immunity and the target of hepatitis C virus NS3/4A protease. J Virol 83, 1299-311 (2009).
28. Agalioti, T. et al. Ordered recruitment of chromatin modifying and general transcription factors to the IFN-beta promoter. Cell 103, 667-78 (2000).
29. Agalioti, T., Chen, G. & Thanos, D. Deciphering the transcriptional histone acetylation code for a human gene. Cell 111, 381-92 (2002).
30. Kato, H. et al. Length-dependent recognition of double-stranded ribonucleic acids by retinoic acid-inducible gene-I and melanoma differentiation-associated gene 5. J Exp Med 205, 1601-10 (2008).
31. Schlee, M. et al. Recognition of 5′ triphosphate by RIG-I helicase requires short blunt double-stranded RNA as contained in panhandle of negative-strand virus. Immunity 31, 25-34 (2009).
32. Schmidt, A. et al. 5′-triphosphate RNA requires base-paired structures to activate antiviral signaling via RIG-I. Proc Natl Acad Sci USA 106, 12067-72 (2009).
33. Myong, S. et al. Cytosolic viral sensor RIG-I is a 5′-triphosphate-dependent translocase on double-stranded RNA. Science 323, 1070-4 (2009).
34. Saito, T. et al. Regulation of innate antiviral defenses through a shared repressor domain in RIG-I and LGP2. Proc Natl Acad Sci USA 104, 582-7 (2007).
35. Gack, M. U. et al. TRIM25 RING-finger E3 ubiquitin ligase is essential for RIG-1-mediated antiviral activity. Nature 446, 916-920 (2007).
36. Gack, M. U., Nistal-Villan, E., Inn, K. S., Garcia-Sastre, A. & Jung, J. U. Phosphorylation-mediated negative regulation of RIG-I anti-viral activity. J. Virol.
37. Oganesyan, G. et al. Critical role of TRAF3 in the Toll-like receptor-dependent and -independent antiviral response. Nature 439, 208-11 (2006).
38. Hacker, H. et al. Specificity in Toll-like receptor signalling through distinct effector functions of TRAF3 and TRAF6. Nature 439, 204-7 (2006).
39. Saha, S. K. et al. Regulation of antiviral responses by a direct and specific interaction between TRAF3 and Cardif. EMBO J. 25, 3257-63 (2006).
40. Guo, B. & Cheng, G. Modulation of the interferon antiviral response by the TBK1/IKKi adaptor protein TANK. J Biol Chem 282, 11817-26 (2007).
41. Ishikawa, H. & Barber, G. N. STING is an endoplasmic reticulum adaptor that facilitates innate immune signalling. Nature 455, 674-8 (2008).
42. Zhong, B. et al. The adaptor protein MITA links virus-sensing receptors to IRF3 transcription factor activation. Immunity 29, 538-50 (2008).
43. Schroder, M., Baran, M. & Bowie, A. G. Viral targeting of DEAD box protein 3 reveals its role in TBK1/IKKepsilon-mediated IRF activation. EMBO J. 27, 2147-57 (2008).
44. Prassas, I. & Diamandis, E. P. Novel therapeutic applications of cardiac glycosides. Nat Rev Drug Discov 7, 926-35 (2008).
45. Thanos, D. & Maniatis, T. Virus induction of human IFN beta gene expression requires the assembly of an enhanceosome. Cell 83, 1091-100 (1995).
46. Hemmi, H. et al. The roles of two IkappaB kinase-related kinases in lipopolysaccharide and double stranded RNA signaling and viral infection. J Exp Med 199, 1641-50 (2004).
47. Du, W., Thanos, D. & Maniatis, T. Mechanisms of transcriptional synergism between distinct virus-inducible enhancer elements. Cell 74, 887-98 (1993).
48. Fitzgerald, K. A. et al. IKKepsilon and TBK1 are essential components of the IRF3 signaling pathway. Nat Immunol 4, 491-6 (2003).
49. Yoneyama, M. et al. The RNA helicase RIG-I has an essential function in double-stranded RNA-induced innate antiviral responses. Nat Immunol 5, 730-7 (2004).
50. Malathi, K., Dong, B., Gale, M., Jr. & Silverman, R. H. Small self-RNA generated by RNase L amplifies antiviral innate immunity. Nature 448, 816-9 (2007).
51. Langer, G. A. Ionic basis of myocardial contractility. Annu Rev Med 28, 13-20 (1977).
52. Miura, D. S. & Biedert, S. Cellular mechanisms of digitalis action. J Clin Pharmacol 25, 490-500 (1985).
53. Sumpter, R., Jr. et al. Regulating intracellular antiviral defense and permissiveness to hepatitis C virus RNA replication through a cellular RNA helicase, RIG-I. J Virol 79, 2689-99 (2005).
54. Morth, J. P. et al. Crystal structure of the sodium-potassium pump. Nature 450, 1043-9. (2007).
55. Shinoda, T., Ogawa, H., Cornelius, F. & Toyoshima, C. Crystal structure of the sodium-potassium pump at 2.4 A resolution. Nature 459, 446-50 (2009).
56. James, P. F. et al. Identification of a specific role for the Na,K-ATPase alpha 2 isoform as a regulator of calcium in the heart. Mol Cell 3, 555-63 (1999).
57. Ohtsubo, M., Noguchi, S., Takeda, K., Morohashi, M. & Kawamura, M. Site-directed mutagenesis of Asp-376, the catalytic phosphorylation site, and Lys-507, the putative ATP-binding site, of the alpha-subunit of Torpedo californica Na+/K(+)-ATPase. Biochim Biophys Acta 1021, 157-60 (1990).
58. Lingrel, J. B. The physiological significance of the cardiotonic steroid/ouabain-binding site of the Na,K-ATPase. Annu Rev Physiol 72, 395-412.
59. Izquierdo, I. Nimodipine and the recovery of memory. Trends Pharmacol Sci 11, 309-10 (1990).
60. Trube, G., Rorsman, P. & Ohno-Shosaku, T. Opposite effects of tolbutamide and diazoxide on the ATP-dependent K+ channel in mouse pancreatic beta-cells. Pflugers Arch 407, 493-9 (1986).
61. Garvin, J. L., Simon, S. A., Cragoe, E. J., Jr. & Mandel, L. J. Phenamil: an irreversible inhibitor of sodium channels in the toad urinary bladder. J Membr Biol 87, 45-54 (1985).
62. Xie, Z. & Cai, T. Na+-K+−-ATPase-mediated signal transduction: from protein interaction to cellular function. Mol Intery 3, 157-68 (2003).
63. Chiu, Y. H., Macmillan, J. B. & Chen, Z. J. RNA polymerase III detects cytosolic DNA and induces type I interferons through the RIG-I pathway. Cell 138, 576-91 (2009).
64. Ablasser, A. et al. RIG-I-dependent sensing of poly(dA:dT) through the induction of an RNA polymerase III-transcribed RNA intermediate. Nat Immunol 10, 1065-72 (2009).
65. Gee, P. et al. Essential role of the N-terminal domain in the regulation of RIG-I ATPase activity. J Biol Chem 283, 9488-96 (2008).
66. Lohman, T. M., Chao, K., Green, J. M., Sage, S. & Runyon, G. T. Large-scale purification and characterization of the Escherichia coli rep gene product. J Biol Chem 264, 10139-47 (1989).
67. Runyon, G. T., Wong, I. & Lohman, T. M. Overexpression, purification, DNA binding, and dimerization of the Escherichia coli uvrD gene product (helicase II). Biochemistry 32, 602-12 (1993).
68. Hoffmann, H. H., Palese, P. & Shaw, M. L. Modulation of influenza virus-replication by alteration of sodium ion transport and protein kinase C activity. Antiviral Res 80, 124-34 (2008).
69. Fagerlund, R., Kinnunen, L., Kohler, M., Julkunen, I. & Melen, K. NF-{kappa}B is transported into the nucleus by importin {alpha}3 and importin {alpha}4. J Biol Chem 280, 15942-51 (2005).
70. Drier, E. A., Huang, L. H. & Steward, R. Nuclear import of the Drosophila Rel protein Dorsal is regulated by phosphorylation. Genes Dev 13, 556-68 (1999).
71. Bhaskar, V., Valentine, S. A. & Courey, A. J. A functional interaction between dorsal and components of the Smt3 conjugation machinery. J Biol Chem 275, 4033-40 (2000).
72. Yao, Y. et al. Neutralization of interferon-alpha/beta-inducible genes and downstream effect in a phase I trial of an anti-interferon-alpha monoclonal antibody in systemic lupus erythematosus. Arthritis Rheum 60, 1785-96 (2009).
73. Zagury, D. et al. IFNalpha kinoid vaccine-induced neutralizing antibodies prevent clinical manifestations in a lupus flare murine model. Proc Natl Acad Sci USA 106, 5294-9 (2009).
74. Meng, Z. et al. Pilot study of huachansu in patients with hepatocellular carcinoma, nonsmall-cell lung cancer, or pancreatic cancer. Cancer 115, 5309-18 (2009).
75. McWhirter, S. M. et al. IFN-regulatory factor 3-dependent gene expression is defective in Tbk1-deficient mouse embryonic fibroblasts. Proc Natl Acad Sci USA 101, 233-8 (2004).
76. Chen, S. et al. A small molecule that directs differentiation of human ESCs into the pancreatic lineage. Nat Chem Biol 5, 258-65 (2009).
77. Honda, K., Takaoka, A. & Taniguchi, T. Type I interferon [corrected] gene induction by the interferon regulatory factor family of transcription factors. Immunity 25, 349-60 (2006).
78. Chiu, Y. H., Macmillan, J. B. & Chen, Z. J. RNA polymerase III detects cytosolic DNA and induces type I interferons through the RIG-I pathway. Cell 138, 576-91 (2009).
79. Ablasser, A. et al. RIG-1-dependent sensing of poly(dA:dT) through the induction of an RNA polymerase III-transcribed RNA intermediate. Nat Immunol 10, 1065-72 (2009).
80. Jacquelin, B. et al. Nonpathogenic SIV infection of African green monkeys induces a strong but rapidly controlled type I IFN response. J Clin Invest 119, 3544-55 (2009).
81. Fujita, T. A nonself RNA pattern: tri-p to panhandle. Immunity 31, 4-5 (2009).
82. Simpson, C. D. et al. Inhibition of the sodium potassium adenosine triphosphatase pump sensitizes cancer cells to anoikis and prevents distant tumor formation. Cancer Res 69, 2739-47 (2009).
83. Cyert, M. S. Regulation of nuclear localization during signaling. J Biol Chem 276, 20805-8 (2001).
84. Yang, et al., Cardiac glycosides inhibit TNF-alpha/NF-kappaB signaling by blocking recruitment of TNF receptor-associated death domin to the TNF receptor. Proc. Natl. Acad. Sci. USA 102, 9631-9636 (2005).
85. Ronnblom, L. & Elkon, K. B. Cytokines as therapeutic targets in SLE. Nat Rev Rheumatol 6, 339-47.
86. Minta, A. & Tsien, R. Y. Fluorescent indicators for cytosolic sodium. J Biol Chem 264, 19449-57 (1989).
87. Ishikawa, S., Fujisawa, G., Okada, K. & Saito, T. Thapsigargin increases cellular free calcium and intracellular sodium concentrations in cultured rat glomerular mesangial cells. Biochem Biophys Res Commun 194, 287-93 (1993).
88. Mori, M. et al. Identification of Ser-386 of interferon regulatory factor 3 as critical target for inducible phosphorylation that determines activation. J Biol Chem 279, 9698-702 (2004).

Content of all patents and other publications identified and listed in the specification is herein incorporated by reference in its entirety.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method for inhibiting gene expression of interferon-beta, the method comprising contacting a cell with a Na⁺, Ca²⁺, or K⁺ ion channel modulator, wherein the modulator is not an amiloride or analog or derivate thereof.

2. The method of claim 1, wherein the modulator is an antiarrhythmic agent.

3. The method of claim 1, wherein the modulator is an inhibitor or antagonist of a Na⁺, Ca²⁺, or K⁺ ion channel.

4. The method of claim 3, wherein the Na⁺ ion channel is a Na⁺/K⁺ ion channel.

5. The method of claim 4, wherein the modulator inhibits the ATPase activity of the Na⁺/K⁺ ion channel.

6. The method of claim 3, wherein the modulator is a calcium channel blocker.

7. The method of claim 3, wherein the modulator is a potassium channel opener.

8. The method of claim 1, wherein the modulator is a cardiac glycoside.

9-16. (canceled)

17. The method of claim 3, further comprising a step of selecting a subject with elevated levels of interferon-beta prior to contacting the cell with the Na⁺, Ca²⁺, or K⁺ ion channel modulator.

18. The method of claim 17, wherein the contact is in a subject, and the subject suffers from an autoimmune disease, neurodegenerative disease, inflammation, an inflammation associated disorder, a disease characterized by inflammation, or a pathogen or non-pathogen infection.

19-23. (canceled)

24. The method of claim 1, wherein the modulator is selected from the group consisting of bufalin; 7β-Hydroxyl bufalin; 3-epi-7β-Hydroxyl bufalin; 1β-Hydroxyl bufalin; 15α-Hydroxyl bufalin; 15β-Hydroxyl bufalin; Telocinobufagin (5-hydroxyl bufalin); 11β-Hydroxyl bufalin; 12β-Hydroxyl bufalin; 1β,7β-Dihydroxyl bufalin; 16α-Hydroxyl bufalin; 7β,16α-Dihydroxyl bufalin; 1β,12β-Dihydroxyl bufalin; resibufogenin; norbufalin; 3-hydroxy-14(15)-en-19-norbufalin-20,22-dienolide; 14-dehydrobufalin; 14,15-epoxy-bufalin; digoxin; ouabain; nimodipine; diazoxide; digitoxigenin; ranolazine; lanatoside C; Strophantin K; uzarigenin; desacetyllanatoside A; actyl digitoxin; desacetyllanatoside C; strophanthoside; scillaren A; proscillaridin A; digitoxose; gitoxin; strophanthidiol; oleandrin; acovenoside A; strophanthidine digilanobioside; strophanthidin-d-cymaroside; digitoxigenin-L-rhamnoside; digitoxigenin theretoside; strophanthidin; digoxigenin-3,12-diacetate; gitoxigenin; gitoxigenin 3-acetate; gitoxigenin-3,16-diacetate; 16-acetyl gitoxigenin; acetyl strophanthidin; ouabagenin; 3-epigoxigenin; neriifolin; acetyhieriifolin cerberin; theventin; somalin; odoroside; honghelin; desacetyl digilanide; calotropin; calotoxin; convallatoxin; oleandrigenin; periplocyrnarin; strophanthidin oxime; strophanthidin semicarbazone; strophanthidinic acid lactone acetate; ernicyrnarin; sarmentoside D; sarverogenin; sarmentoside A; sarmentogenin; proscillariditi; marinobufagenin; Amiodarone; Dofetilide; Sotalol; Ibutilide; Azimilide; Bretylium; Clofilium; E-4031; Nifekalant; Tedisamil; Sematilide; Ampyra; apamin; charybdotoxin; 1-EBIO; NS309; CyPPA; GPCR antagonists; ifenprodil; glibenclamide; tolbutamide; diazoxide; pinacidil; halothane; tetraethylammonium; 4-aminopyridine; dendrotoxins; retigabine; 4-aminopyridine; 3,4-diaminopyridine; diazoxide; Minoxidil; Nicorandi; Retigabine; Flupirtine; Quinidine; Procainamide; Disopyramide; Lidocaine; Phenyloin; Mexiletine; Flecamide; Propafenone; Moricizine; atenolol; ropranolol; Esmolol; Timolol; Metoprolol; Atenolol; Bisoprolol; Amiodarone; Sotalol; Ibutilide; Dofetilide; Adenosine; Nifedipine; δ-conotoxin; κ-conotoxin; μ-conotoxin; ω-conotoxin; ω-conotoxin GVIA; ω-conotoxin ω-conotoxin CNVIIA; ω-conotoxin CVIID; ω-conotoxin AM336; cilnidipine; L-cysteine derivative 2A; ω-agatoxin IVA; N,N-dialkyl-dipeptidyl-amines; SNX-111 (Ziconotide); caffeine; lamotrigine; 202W92 (structural analog of lamotrigine); phenyloin; carbamazepine; 1,4-dihydro-2,6-dimethyl-5-nitro-4-[thieno[3,2-c]-pyridin-3-yl]-3-pyridinecarboxylic acid, 1-phenylethyl ester; 1,4-dihydro-2,6-dimethyl-5-nitro-4-[thieno[3,2-c]-pyridin-3-yl]-3-pyridinecarboxylic acid, 1-methyl-2-propynyl ester; 1,4-dihydro-2,6-dimethyl-5-nitro-4-[3,2-c]pyridin-3-yl]-3-pyridinecarboxylic acid, cyclopropylmethyl ester; 1,4-dihydro-2,6-dimethyl-5-nitro-4-[thieno(3,2-c)pyridin-3-yl]-3-pyridinecarboxylic acid, butyl ester; (S)-1,4-Dihydro-2,6-dimethyl-5-nitro-4-[thieno[3,2c]pyridin-3-yl]-3-pyridinecarboxylic acid, 1-methylpropyl ester; 1,4-Dihydro-2,6-dimethyl-5-nitro-4-thieno[3,2-c]pyridin-3-yl]-3-pyridinecarboxylic acid, methyl ester; 1,4-Dihydro-2,6-dimethyl-5-nitro-4-[thieno[3,2-c]pyridin-3-yl]-3-pyridinecarboxylic acid, 1-methylethyl ester; 1,4-Dihydro-2,6-dimethyl-5-nitro-4-thieno[3,2-c]pryridin-3-yl]-3-pyridinecarboxylic acid, 2-propynyl ester; 1,4-Dihydro-2,6-dimethyl-5-nitro-4-[thieno[3,2-c]pyridin-3-yl]-3-pyridinecarboxylic acid, 1-methyl-2propynyl ester; 1,4-Dihydro-2,6-dimethyl-5-nitro-4-[thieno[3,2-c]pyridin-3-yl]-3-pyridinecarboxylic acid, 2-butynyl este; 1,4-Dihydro-2,6-dimethyl-5-nitro-4-[thieno[3,2-c]pyridin-3-yl]-3-pyridinecarboxylic acid, 1-methyl-2butynyl este; 1,4-Dihydro-2,6-dimethyl-5-nitro-4-[thieno[3,2-c]pyridin-3-yl]-3-pyridinecarboxylic acid, 2,2-dimethylpropyl ester; 1,4-Dihydro-2,6-dimethyl-5-nitro-4-thieno[3,2-c]pyridin-3-yl]-3-pyridinecarboxylic acid, 3-butynyl ester; 1,4-Dihydro-2,6-dimethyl-5-nitro-4-[thieno3,2-c]pyridin-3-yl]-3-pyridinecarboxylic acid, 1,1-dimethyl-2propynyl ester; 1,4-Dihydro-2,6-dimethyl-5-nitro-4-[thieno-3,2-c]pyridin-3-yl-3-pyridinecarboxylic acid, 1,2,2-trimethylpropyl ester; R(+)-1,4-Dihydro-2,6-dimethyl-5-nitro-4[thieno[3,2-c]pyridin-3-yl]-3-pyridinecarboxylic (2Amethyl-1-phenylpropyl) ester; S-(−)-1,4-Dihydro-2,6-dimethyl-5-nitro-4[thieno[3,2-c]pyridin-3-yl]-3-pyridinecarboxylic acid, 2-methyl-1-phenylpropyl ester; 1,4-Dihydro-2,6-dimethyl-5-nitro-4-[thieno[3,2c]-pyridin-3-yl]-3-pyridinecarboxylic acid, 1-methylphenylethyl ester; 1,4-Dihydro-2,6-dimethyl-5-nitro-4-[thieno[3,2-c]pyridin-3-yl]-3-pyridinecarboxylic acid, 1-phenylethyl ester; 1,4-Dihydro-2,6-dimethyl-5-nitro-4-[thieno[3,2c]-pyridin-3-yl]-3-pyridinecarboxylic acid, (1-phenylpropyl)ester; 1,4-Dihydro-2,6-dimethyl-5-nitro-4-[thieno[3,2c]-pyridin-3-yl]-3-pyridinecarboxylic acid, (4-methoxyphenyl)methyl ester; 1,4-Dihydro-2,6-dimethyl-5-nitro-4-[thieno[3,2c]-pyridin-3-yl]-3-pyridinecarboxylic acid, 1-methyl-2-phenylethyl ester; 1,4-Dihydro-2,6-dimethyl-5-nitro-4-[thieno[3,2c]-pyridin-3-yl]-3-pyridinecarboxylic acid, 2-phenylpropyl ester; 1,4-Dihydro-2,6-dimethyl-5-nitro-4-[thieno[3,2c]-pyridin-3-yl]-3-pyridinecarboxylic acid, phenylmethyl ester; 1,4-Dihydro-2,6-dimethyl-5-nitro-4-[thieno[3,2c]-pyridin-3-yl]-3-pyridinecarboxylic acid, 2-phenoxyethyl ester; 1,4-Dihydro-2,6-dimethyl-5-nitro-4-thieno-3,2-c]pyridin-3-yl]-3-pyridinecarboxylic acid, 3-phenyl-2propynyl este; 1,4-Dihydro-2,6-dimethyl-5-nitro-4-[thieno[3,2c]-pyridin-3-yl]-3-pyridinecarboxylic acid, 2-methoxy-2-phenylethyl ester; (5)-1,4-Dihydro-2,6-dimethyl-5-nitro-4-[thieno[3,2-c]pyridin-3-yl]-3-pyridinecarboxylic acid, 1-phenylethyl este; (R)-1,4-Dihydro-2,6-dimethyl-5-nitro-4-[thieno[3,2-c]pyridin-3-yl]-3-pyridinecarboxylic acid, 1-phenylethyl este; 1,4-Dihydro-2,6-dimethyl-5-nitro-4-[thieno[3,2c]-pyridin-3-yl]-3-pyridinecarboxylic acid, cyclopropylmethyl ester; 1,4-Dihydro-2,6-dimethyl-5-nitro-4-thieno[3,2-c]pyridin-3-yl]-3-pyridinecarboxylic acid, 1-cyclopropylethyl este; 1,4-Dihydro-2,6-dimethyl-5-nitro-4-[thieno[3,2c]-Pyridin-3-yl]-3-pyridinecarboxylic acid, 2-cyanoethyl ester; 1,4-Dihydro-4-(2-[(5-[4-(2-methoxyphenyl)-1-1piperazinyl]pentyl]-3-furanyl)-2,6-dimethyl-5-nitro-3-pyridinecarboxylic acid, methyl ester; 4-(4-Benzofurazanyl)-1,4-dihydro-2,6-dimethyl-5-nitro-3-pyridinecarboxylic acid, {4-[4-(2-methoxyphenyl)-1-piperazinyl]butyl}ester; 1,4-Dihydro-2,6-dimethyl-5-nitro-4-(3-pyridinyl)-3-pyridinecarboxylicacid, {4-[4-(2-pyrimidinyl)-1-piperazinyl]butyl}ester; 4-(3-Furanyl)-1,4-dihydro-2,6-dimethyl-5-nitro-3pyridinecarboxylic acid, {2-[4-(2-methoxyphenyl)-lpiperazinyl]ethyl}ester; 4-(3-Furanyl)-1,4-dihydro-2,6-dimethyl-5-nitro-3pyridinecarboxylic acid, {2-[4-(2-pyrimidinyl)-lpiperazinyl]ethyl}ester; 1,4-Dihydro-2,6-dimethyl-4-(1-methyl-1H-pyrrol-2-yl)-5-nitro-3-pyridinecarboxylic acid, {4-[4-(2-methoxyphenyl)1-piperazinyl]butyl}ester; 1,4-Dihydro-2,6-dimethyl-4-(1-methyl-1H-pyrrol-2-yl)-5-nitro-3-pyridinecarboxylic acid, {4-[4-(2pyrimidinyl)-1-piperazinyl]butyl}ester; 1,4-Dihydro-2,6-dimethyl-5-nitro-4-(3-thienyl)-3-pyridinecarboxylic acid, {2-[4-(2-methoxyphenyl)-1-piperazinyl]ethyl}ester; 1,4-Dihydro-2,6-dimethyl-5-nitro-4-(3-thienyl)-3-pyridinecarboxylic acid, {2-[4-(2-pyrimidinyl)-1-piperazinyl]ethyl}ester; 4-(3-Furanyl)-1,4-dihydro-2,6-dimethyl-5-nitro-3-pyridinecarboxylic acid, {4-[4-(2-pyrimidinyl)-1-piperazinyl]butyl}ester; (4-(2-Furanyl)-1,4-dihydro-2,6-dimethyl-5-nitro-3-pyridinecarboxylic acid, {4-[4-(2-pyrimidinyl)-1-piperazinyl]butyl}ester; 1,4-Dihydro-2,6-dimethyl-5-nitro-4-(2-thienyl)-3-pyridinecarboxylic acid, {2-[4-(2-methoxyphenyl)-1-piperazinyl]ethyl}ester; 1,4-Dihydro-2,6-dimethyl-4-(1-methyl-1H-pyrrol-2-yl)-5-nitro-3-pyridinecarboxylic acid, {2-[4-(2-methoxyphenyl)-1-piperazinyl]ethyl}ester; 1,4-Dihydro-2,6-dimethyl-4-(1-methyl-1H-pyrrol-2-yl)-5-nitro-3-pyridinecarboxylic acid, {2-[4-(2pyrimidinyl) 1-piperazinyl]ethyl}ester; A-803467; and a pharmaceutically acceptable salt, ester, amide, or prodrug thereof.

25. A method for treating systemic lupus erythematosus (SLE), in a subject, the method comprising: administering an effective amount of a Na⁺, Ca²⁺, or K⁺ ion channel modulator to a subject, wherein the modulator is not an amiloride or analog or derivate thereof.

26-46. (canceled)

47. A method for treating neurodegenerative disease in a subject, the method comprising: administering an effective amount of a Na⁺, Ca²⁺, or K⁺ ion channel modulator to a subject, wherein the modulator is not an amiloride or analog or derivate thereof.

48-65. (canceled)

66. A method for treating pathogenic or non-pathogenic infection in a subject, the method comprising: administering an effective amount of a Na⁺, Ca²⁺, or K⁺ ion channel modulator to a subject, wherein the modulator is not an amiloride or analog or derivate thereof.

67-85. (canceled)

86. A method for treating inflammation, an inflammation associated disorder, or a disease characterized by inflammation in a subject, the method comprising: administering an effective amount of bufalin or analog or derivate thereof.

87-92. (canceled)

93. The method of claim 86, wherein bufalin or analog or derivate thereof is selected from the group consisting of bufalin; 7β-Hydroxyl bufalin; 3-epi-7β-Hydroxyl bufalin; 1β-Hydroxyl bufalin; 15α-Hydroxyl bufalin; 15β-Hydroxyl bufalin; Telocinobufagin (5-hydroxyl bufalin); 3-epi-Telocinobufagin; 3-epi-Bufalin-3-O-β-d-glucoside; 11β-Hydroxyl bufalin; 12β-Hydroxyl bufalin; 1β,7β-Dihydroxyl bufalin; 16α-Hydroxyl bufalin; 7β,16α-Dihydroxyl bufalin; 1β,12β-Dihydroxyl bufalin; resibufogenin; norbufalin; 3-hydroxy-14(15)-en-19-norbufalin-20,22-dienolide; 14-dehydrobufalin; bufotalin; arenobufagin; cinobufagin; marinobufagenin; proscillaridin; scillroside; and 14,15-epoxy-bufalin.


SEQUENCE LISTING (63 sequences)

SEQ ID NO: 1 - DNA, 20 bp, Artificial Sequence
  Human IFNb primer
gctgcagctgcttaatctcc

SEQ ID NO: 2 - DNA, 20 bp, Artificial Sequence
  Human IFNb primer
tcctccaaattgctctcctg

SEQ ID NO: 3 - DNA, 20 bp, Artificial Sequence
  Human Cxcl10 primer
aaggatggaccacacagagg

SEQ ID NO: 4 - DNA, 20 bp, Artificial Sequence
  Human Cxcl10 primer
tggaagatgggaaaggtgag

SEQ ID NO: 5 - DNA, 20 bp, Artificial Sequence
  Human GAPDH primer
ctgacttcaacagcgacacc

SEQ ID NO: 6 - DNA, 20 bp, Artificial Sequence
  Human GAPDH primer
ggtggtccaggggtcttact

SEQ ID NO: 7 - DNA, 20 bp, Artificial Sequence
  Human IFNa8 primer
acccaggttaagggtcatcc

SEQ ID NO: 8 - DNA, 20 bp, Artificial Sequence
  Human IFNa8 primer
atcaaggccctcctgttacc

SEQ ID NO: 9 - DNA, 20 bp, Artificial Sequence
  Human TNF primer
cctgtgaggaggacgaacat

SEQ ID NO: 10 - DNA, 20 bp, Artificial Sequence
  Human TNF primer
aggccccagtttgaattctt

SEQ ID NO: 11 - DNA, 20 bp, Artificial Sequence
  Human RIG-I primer
caaaccagaggcagaggaag

SEQ ID NO: 12 - DNA, 20 bp, Artificial Sequence
  Human RIG-I primer
ccaaggctttgcactttctg

SEQ ID NO: 13 - DNA, 20 bp, Artificial Sequence
  Human ISG15 primer
tgtcggtgtcagagctgaag

SEQ ID NO: 14 - DNA, 20 bp, Artificial Sequence
  Human ISG15 primer
gcccttgttattcctcacca

SEQ ID NO: 15 - DNA, 20 bp, Artificial Sequence
  Human CCL5 primer
cgctgtcatcctcattgcta

SEQ ID NO: 16 - DNA, 20 bp, Artificial Sequence
  Human CCL5 primer
tgtactcccgaacccatttc

SEQ ID NO: 17 - DNA, 20 bp, Artificial Sequence
  Human IFIT2 primer
attgccaaaatgcgactttc

SEQ ID NO: 18 - DNA, 20 bp, Artificial Sequence
  Human IFIT2 primer
atttcagctccctttcagca

SEQ ID NO: 19 - DNA, 20 bp, Artificial Sequence
  Human IL8 primer
ctgcgccaacacagaaatta

SEQ ID NO: 20 - DNA, 20 bp, Artificial Sequence
  Human IL8 primer
attgcatctggcaaccctac

SEQ ID NO: 21 - DNA, 20 bp, Artificial Sequence
  Human OASL primer
acctgaggatggagcagaga

SEQ ID NO: 22 - DNA, 20 bp, Artificial Sequence
  Human OASL primer
cagcttagttggccgatgtt

SEQ ID NO: 23 - DNA, 20 bp, Artificial Sequence
  Human JUN primer
cgaaaaaggaagctggagag

SEQ ID NO: 24 - DNA, 20 bp, Artificial Sequence
  Human JUN primer
ccgacggtctctcttcaaaa

SEQ ID NO: 25 - DNA, 20 bp, Artificial Sequence
  Human PRDM4 primer
gactgggagggaagtgtcaa

SEQ ID NO: 26 - DNA, 20 bp, Artificial Sequence
  Human PRDM4 primer
gctgtgtcccaatccattct

SEQ ID NO: 27 - DNA, 20 bp, Artificial Sequence
  Human TMEM60 primer
ggatgagaaagcaccttgga

SEQ ID NO: 28 - DNA, 20 bp, Artificial Sequence
  Human TMEM60 primer
agcaaggcccataaaggaat

SEQ ID NO: 29 - DNA, 20 bp, Artificial Sequence
  Human OVGP1 primer
gtgtggacattggacatgga

SEQ ID NO: 30 - DNA, 20 bp, Artificial Sequence
  Human OVGP1 primer
cctgggggcaaaatcttact

SEQ ID NO: 31 - DNA, 20 bp, Artificial Sequence
  Human LINS1 primer
cctggatttgcttgagcttc

SEQ ID NO: 32 - DNA, 20 bp, Artificial Sequence
  Human LINS1 primer
gcattaaggcaggcacagat

SEQ ID NO: 33 - DNA, 20 bp, Artificial Sequence
  Human TXNIP1 primer
gccacacttaccttgccaat

SEQ ID NO: 34 - DNA, 20 bp, Artificial Sequence
  Human TXNIP1 primer
ttggatccaggaacgctaac

SEQ ID NO: 35 - DNA, 20 bp, Artificial Sequence
  Human PPP1R15A primer
gatcagccgaggatgaaaga

SEQ ID NO: 36 - DNA, 20 bp, Artificial Sequence
  Human PPP1R15A primer
tcctcagcagcttcctcttc

SEQ ID NO: 37 - DNA, 22 bp, Artificial Sequence
  Human CITED2 primer
cgacgaggaagttcttatgtcc

SEQ ID NO: 38 - DNA, 20 bp, Artificial Sequence
  Human CITED2 primer
aattcacgccgaagaagttg

SEQ ID NO: 39 - DNA, 23 bp, Artificial Sequence
  Human PRMT6 primer
gaccacatacatcatagggtgct

SEQ ID NO: 40 - DNA, 20 bp, Artificial Sequence
  Human PRMT6 primer
gggctaggctcagaaacctc

SEQ ID NO: 41 - DNA, 20 bp, Artificial Sequence
  Mouse IFNb primer
ccctatggagatgacggaga

SEQ ID NO: 42 - DNA, 20 bp, Artificial Sequence
  Mouse IFNb primer
ctgtctgctggtggagttca

SEQ ID NO: 43 - DNA, 20 bp, Artificial Sequence
  Mouse Cxcl10 primer
tcatcctgctgggtctgagt

SEQ ID NO: 44 - DNA, 21 bp, Artificial Sequence
  Mouse Cxcl10 primer
ttttggctaaacgctttcatt

SEQ ID NO: 45 - DNA, 20 bp, Artificial Sequence
  Mouse B-Actin primer
cctctatgccaacacagtgc

SEQ ID NO: 46 - DNA, 20 bp, Artificial Sequence
  Mouse B-Actin primer
acatctgctggaaggtggac

SEQ ID NO: 47 - DNA, 20 bp, Artificial Sequence
  Mouse IRF7 primer
tgcaaggtgtactgggaggt

SEQ ID NO: 48 - DNA, 20 bp, Artificial Sequence
  Mouse IRF7 primer
tcaccaggatcagggtcttc

SEQ ID NO: 49 - DNA, 20 bp, Artificial Sequence
  Mouse RIG-I primer
agagccagcggagataacaa

SEQ ID NO: 50 - DNA, 20 bp, Artificial Sequence
  Mouse RIG-I primer
ccttgatcatgttcgccttt

SEQ ID NO: 51 - DNA, 20 bp, Artificial Sequence
  Mouse Stat1 primer
gaccacctctcttcctgtcg

SEQ ID NO: 52 - DNA, 20 bp, Artificial Sequence
  Mouse Stat1 primer
tgccaactcaacacctctga

SEQ ID NO: 53 - DNA, 20 bp, Artificial Sequence
  Mouse trex1 primer
gagcaaagctgagctggaag

SEQ ID NO: 54 - DNA, 20 bp, Artificial Sequence
  Mouse trex1 primer
gctgctagcttgttccaagg

SEQ ID NO: 55 - DNA, 20 bp, Artificial Sequence
  Mouse CCL5 primer
ccctcaccatcatcctcact

SEQ ID NO: 56 - DNA, 20 bp, Artificial Sequence
  Mouse CCL5 primer
gggaagcgtatacagggtca

SEQ ID NO: 57 - DNA, 30 bp, Artificial Sequence
  Sendai virus NP primer
gctcactcattagacacagataagcagcac

SEQ ID NO: 58 - DNA, 27 bp, Artificial Sequence
  Sendai virus NP primer
gaaaagcggactcttgttgaccatagg

SEQ ID NO: 59 - DNA, 20 bp, Artificial Sequence
  Sendai virus L primer
tgatgtcaatgggcagagag

SEQ ID NO: 60 - DNA, 23 bp, Artificial Sequence
  Sendai virus L primer
catgcagtacaacttgatcatcc

SEQ ID NO: 61 - protein, 40 aa, Artificial Sequence
  Cardiac glycosides binding sites in human ATP1a1 protein
SMLLWIGAILCFLAYSIQAATEEEPQNDNLYLGVVLSAVV

SEQ ID NO: 62 - protein, 40 aa, Artificial Sequence
  Cardiac glycosides binding sites in mouse and rat ATP1a1 protein
SMLLWIGAILCFLAYGIRSATEEEPPNDDLYLGVVLSAVV

SEQ ID NO: 63 - protein, 40 aa, Artificial Sequence; identical to SEQ ID NO: 61
  Cardiac glycosides binding sites in human, mouse and rat ATP1a3 protein